SEQUENCE LISTING (68 sequences)

SEQ ID NO: 1 - DNA, 22 bp, Artificial Sequence
  Description of Artificial Sequence Synthetic primer
tcagcaaggacacatcctctaa

SEQ ID NO: 2 - DNA, 21 bp, Artificial Sequence
  Description of Artificial Sequence Synthetic primer
taaggatcctcattggactgc

SEQ ID NO: 3 - DNA, 1602 bp, Artificial Sequence
  Description of Artificial Sequence Synthetic polynucleotide
atgaagagccagacccaggtgttcatcttcctgctgctgtgcgtgagcggcgcccacggcgacatccagatgacccagagcaccagcagcctgagcgccagcctgggcgacagggtgaccatcagctgcagcgccagccagggcatcaacaactacctgaactggtaccagcagaagcccgacggcaccgtggagctgctgatctactacaccagcaccctgcagagcggcgtgcccagcaggttcagcggcagcggcagcggcaccgactacagcctgaccatcagcaacctggagcccgaggacatcggcacctactactgccagcagtacagcaagctgcccaggaccttcggcggcggcaccaagctggagatcaagggtggcggtggctccggcggtggtgggtcgggtggcggcggatctagccaggtgcagctgcagcagagcggcagcgagctgatgatgcccggcgccagcgtgaagatcagctgcaaggccaccggctacaccttcagcaactactggatcgagtgggtgaagcagaggcccggccacggcctggagtggatcggcgagatcctgcccggcaccggcaggaccatctacaacgagaagttcaagggcaaggccaccttcaccgccgacatcagcagcaacaccgtgcagatgcagctgagcagcctgaccagcgaggacagcgccgtgtactactgcgccaggagggactactacggcaacttctactacgccatggactactggggccagggcaccagcgtgaccgtgagcagcggtggcggtggctccggcggtggtgggtcgcaggttactctgaaagagtctggccctgggatattgcagccctcccagaccctcagtctgacttgttctttctctgggttttcactgaggacttctggtatgggtgtaggctggattcgtcagccttcagggaagggtctagagtggctggcacacatttggtgggatgatgacaagcgctataatccagccctgaagagccgactgacaatctccaaggatacctccagcaaccaggtattcctcaaaatcgccagtgtggacactgcagatactgccacatactactgtgctcaaataaaccccgcctggtttgcttactggggccaagggactctggtcactgtctctgccggtggcggtggctccggcggtggtgggtcgggtggcggcggatctgacactgtgctgacccaatctccagcttctttggctgtgtctctagggcagagggccaccatctcctgcaaggccagccaaagtgttgattttgatggtgatagttttatgaactggtaccaacagaaaccaggacagccacccaaactcctcatctatactacatccaatctagaatctgggatcccagccaggtttagtgccagtgggtctgggacagacttcaccctcaacatccatcctgtggaggaggaggatactgcaacctattactgtcagcaaagtaatgaggatccgtacacgttcggaggggggaccaagctggaaataaaaggtaagcctatccctaaccctctcctcggtctcgattctacgtaa

SEQ ID NO: 4 - protein, 533 aa, Artificial Sequence
  Description of Artificial Sequence Synthetic polypeptide
MKSQTQVFIFLLLCVSGAHGDIQMTQSTSSLSASLGDRVTISCSASQGINNYLNWYQQKPDGTVELLIYYTSTLQSGVPSRFSGSGSGTDYSLTISNLEPEDIGTYYCQQYSKLPRTFGGGTKLEIKGGGGSGGGGSGGGGSSQVQLQQSGSELMMPGASVKISCKATGYTFSNYWIEWVKQRPGHGLEWIGEILPGTGRTIYNEKFKGKATFTADISSNTVQMQLSSLTSEDSAVYYCARRDYYGNFYYAMDYWGQGTSVTVSSGGGGSGGGGSQVTLKESGPGILQPSQTLSLTCSFSGFSLRTSGMGVGWIRQPSGKGLEWLAHIWWDDDKRYNPALKSRLTISKDTSSNQVFLKIASVDTADTATYYCAQINPAWFAYWGQGTLVTVSAGGGGSGGGGSGGGGSDTVLTQSPASLAVSLGQRATISCKASQSVDFDGDSFMNWYQQKPGQPPKLLIYTTSNLESGIPARFSASGSGTDFTLNIHPVEEEDTATYYCQQSNEDPYTFGGGTKLEIKGKPIPNPLLGLDST

SEQ ID NO: 5 - protein, 499 aa, Artificial Sequence
  Description of Artificial Sequence Synthetic polypeptide
DIQMTQSTSSLSASLGDRVTISCSASQGINNYLNWYQQKPDGTVELLIYYTSTLQSGVPSRFSGSGSGTDYSLTISNLEPEDIGTYYCQQYSKLPRTFGGGTKLEIKGGGGSGGGGSGGGGSSQVQLQQSGSELMMPGASVKISCKATGYTFSNYWIEWVKQRPGHGLEWIGEILPGTGRTIYNEKFKGKATFTADISSNTVQMQLSSLTSEDSAVYYCARRDYYGNFYYAMDYWGQGTSVTVSSGGGGSGGGGSQVTLKESGPGILQPSQTLSLTCSFSGFSLRTSGMGVGWIRQPSGKGLEWLAHIWWDDDKRYNPALKSRLTISKDTSSNQVFLKIASVDTADTATYYCAQINPAWFAYWGQGTLVTVSAGGGGSGGGGSGGGGSDTVLTQSPASLAVSLGQRATISCKASQSVDFDGDSFMNWYQQKPGQPPKLLIYTTSNLESGIPARFSASGSGTDFTLNIHPVEEEDTATYYCQQSNEDPYTFGGGTKLEIK

SEQ ID NO: 6 - protein, 123 aa, Artificial Sequence
  Description of Artificial Sequence Synthetic polypeptide
SQVQLQQSGSELMMPGASVKISCKATGYTFSNYWIEWVKQRPGHGLEWIGEILPGTGRTIYNEKFKGKATFTADISSNTVQMQLSSLTSEDSAVYYCARRDYYGNFYYAMDYWGQGTSVTVSS

SEQ ID NO: 7 - protein, 107 aa, Artificial Sequence
  Description of Artificial Sequence Synthetic polypeptide
DIQMTQSTSSLSASLGDRVTISCSASQGINNYLNWYQQKPDGTVELLIYYTSTLQSGVPSRFSGSGSGTDYSLTISNLEPEDIGTYYCQQYSKLPRTFGGGTKLEIK

SEQ ID NO: 8 - protein, 118 aa, Artificial Sequence
  Description of Artificial Sequence Synthetic polypeptide
QVTLKESGPGILQPSQTLSLTCSFSGFSLRTSGMGVGWIRQPSGKGLEWLAHIWWDDDKRYNPALKSRLTISKDTSSNQVFLKIASVDTADTATYYCAQINPAWFAYWGQGTLVTVSA

SEQ ID NO: 9 - protein, 111 aa, Artificial Sequence
  Description of Artificial Sequence Synthetic polypeptide
DTVLTQSPASLAVSLGQRATISCKASQSVDFDGDSFMNWYQQKPGQPPKLLIYTTSNLESGIPARFSASGSGTDFTLNIHPVEEEDTATYYCQQSNEDPYTFGGGTKLEIK

SEQ ID NO: 10 - protein, 5 aa, Artificial Sequence
  Description of Artificial Sequence Synthetic peptide
NYWIE

SEQ ID NO: 11 - protein, 17 aa, Artificial Sequence
  Description of Artificial Sequence Synthetic peptide
EILPGTGRTIYNEKFKG

SEQ ID NO: 12 - protein, 13 aa, Artificial Sequence
  Description of Artificial Sequence Synthetic peptide
RDYYGNFYYAMDY

SEQ ID NO: 13 - protein, 11 aa, Artificial Sequence
  Description of Artificial Sequence Synthetic peptide
SASQGINNYLN

SEQ ID NO: 14 - protein, 7 aa, Artificial Sequence
  Description of Artificial Sequence Synthetic peptide
YTSTLQS

SEQ ID NO: 15 - protein, 9 aa, Artificial Sequence
  Description of Artificial Sequence Synthetic peptide
QQYSKLPRT

SEQ ID NO: 16 - protein, 7 aa, Artificial Sequence
  Description of Artificial Sequence Synthetic peptide
TSGMGVG

SEQ ID NO: 17 - protein, 16 aa, Artificial Sequence
  Description of Artificial Sequence Synthetic peptide
HIWWDDDKRYNPALKS

SEQ ID NO: 18 - protein, 8 aa, Artificial Sequence
  Description of Artificial Sequence Synthetic peptide
INPAWFAY

SEQ ID NO: 19 - protein, 15 aa, Artificial Sequence
  Description of Artificial Sequence Synthetic peptide
KASQSVDFDGDSFMN

SEQ ID NO: 20 - protein, 7 aa, Artificial Sequence
  Description of Artificial Sequence Synthetic peptide
TTSNLES

SEQ ID NO: 21 - protein, 9 aa, Artificial Sequence
  Description of Artificial Sequence Synthetic peptide
QQSNEDPYT

SEQ ID NO: 22 - protein, 5 aa, Artificial Sequence
  Description of Artificial Sequence Synthetic peptide
GGGGS

SEQ ID NO: 23 - protein, 4 aa, Artificial Sequence
  Description of Artificial Sequence Synthetic peptide
GGGS

SEQ ID NO: 24 - protein, 2 aa, Artificial Sequence
  Description of Artificial Sequence Synthetic peptide
GG

SEQ ID NO: 25 - protein, 18 aa, Artificial Sequence
  Description of Artificial Sequence Synthetic peptide
KESGSVSSEQLAQFRSLD

SEQ ID NO: 26 - protein, 14 aa, Artificial Sequence
  Description of Artificial Sequence Synthetic peptide
EGKSSGSGSESKST

SEQ ID NO: 27 - protein, 12 aa, Artificial Sequence
  Description of Artificial Sequence Synthetic peptide
GSAGSAAGSGEF

SEQ ID NO: 28 - protein, 5 aa, Artificial Sequence
  Description of Artificial Sequence Synthetic peptide
EAAAK

SEQ ID NO: 29 - protein, 5 aa, Artificial Sequence
  Description of Artificial Sequence Synthetic peptide
EAAAR

SEQ ID NO: 30 - protein, 5 aa, Artificial Sequence
  Description of Artificial Sequence Synthetic peptide
PAPAP

SEQ ID NO: 31 - protein, 12 aa, Artificial Sequence
  Description of Artificial Sequence Synthetic peptide
AEAAAKEAAAKA

SEQ ID NO: 32 - protein, 120 aa, Artificial Sequence
  Description of Artificial Sequence Synthetic polypeptide
MQVQLLESGAELARPGASVKMSCKASGYTFTRYTMHWVKQRPGQGLEWIGYINPSRGYTNYNQKFKDKATLTTDKSSSTAYMQLSSLTSEDSAVYYCAGYYDDHYCLDYWGQGTLVTVSS

SEQ ID NO: 33 - protein, 107 aa, Artificial Sequence
  Description of Artificial Sequence Synthetic polypeptide
DIVMTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEIKR

SEQ ID NO: 34 - protein, 5 aa, Artificial Sequence
  Description of Artificial Sequence Synthetic peptide
RYTMH

SEQ ID NO: 35 - protein, 17 aa, Artificial Sequence
  Description of Artificial Sequence Synthetic peptide
YINPSRGYTNYNQKFKD

SEQ ID NO: 36 - protein, 10 aa, Artificial Sequence
  Description of Artificial Sequence Synthetic peptide
YYDDHYCLDY

SEQ ID NO: 37 - protein, 10 aa, Artificial Sequence
  Description of Artificial Sequence Synthetic peptide
SASSSVSYMN

SEQ ID NO: 38 - protein, 7 aa, Artificial Sequence
  Description of Artificial Sequence Synthetic peptide
DTSKLAS

SEQ ID NO: 39 - protein, 9 aa, Artificial Sequence
  Description of Artificial Sequence Synthetic peptide
QQWSSNPFT

SEQ ID NO: 40 - protein, 127 aa, Artificial Sequence
  Description of Artificial Sequence Synthetic polypeptide
QVKLQQWGEGLLQPSETLSRTCVVSGGSISGYYYWTWIRQTPGRGLEWIGHIYGNGATTNYNPSLKSRVTISKDTSKNQFFLNLNSVTDADTAVYYCARGPRPDCTTICYGGWVDVWGPGDLVTVSS

SEQ ID NO: 41 - protein, 108 aa, Artificial Sequence
  Description of Artificial Sequence Synthetic polypeptide
AYELTQPPSVSVSPGQTARITCGGDNSRNEYVHWYQQKPARAPILVIYDDSDRPSGIPERFSGSKSGNTATLTINGVEAGDEADYYCQVWDRASDHPVFGGGTRVTVL

SEQ ID NO: 42 - protein, 129 aa, Artificial Sequence
  Description of Artificial Sequence Synthetic polypeptide
EVQLVESGGGLVQPGGSLRVSCAVSGFTFSDHYMYWFRQAPGKGPEWVGFIRNKPNGGTTEYAASVKDRFTISRDDSKSIAYLQMSSLKIEDTAVYYCTTSYISHCRGGVCYGGYFEFWGQGALVTVSS

SEQ ID NO: 43 - protein, 112 aa, Artificial Sequence
  Description of Artificial Sequence Synthetic polypeptide
EVVMTQSPLSLPITPGEPASISCRSSQSLKHSNGDTFLSWYQQKPGQPPRLLIYKVSNRDSGVPDRFSGSGAGTDFTLKISAVEAEDVGVYFCGQGTRTPPTFGGGTKVEIK

SEQ ID NO: 44 - protein, 127 aa, Artificial Sequence [sequence omitted]

SEQ ID NO: 45 - protein, 110 aa, Artificial Sequence
  Description of Artificial Sequence Synthetic polypeptide
ESVLTQPPSVSGAPGQKVTISCTGSTSNIGGYDLHWYQQLPGTAPKLLIYDINKRPSGISDRFSGSKSGTAASLAITGLQTEDEADYYCQSYDSSLNAQVFGGGTRLTVL

SEQ ID NO: 46 - protein, 6 aa, Artificial Sequence
  Description of Artificial Sequence Synthetic peptide
GYYYWT

SEQ ID NO: 47 - protein, 17 aa, Artificial Sequence
  Description of Artificial Sequence Synthetic peptide
HIYGNGATTNYNPSLKS

SEQ ID NO: 48 - protein, 28 aa, Artificial Sequence
  Description of Artificial Sequence Synthetic peptide
GPRPDCTTICYGGWVDVWGPGDLVTVSS

SEQ ID NO: 49 - protein, 11 aa, Artificial Sequence
  Description of Artificial Sequence Synthetic peptide
GGDNSRNEYVH

SEQ ID NO: 50 - protein, 7 aa, Artificial Sequence
  Description of Artificial Sequence Synthetic peptide
DDSDRPS

SEQ ID NO: 51 - protein, 11 aa, Artificial Sequence
  Description of Artificial Sequence Synthetic peptide
QVWDRASDHPV

SEQ ID NO: 52 - protein, 5 aa, Artificial Sequence
  Description of Artificial Sequence Synthetic peptide
DHYMY

SEQ ID NO: 53 - protein, 19 aa, Artificial Sequence
  Description of Artificial Sequence Synthetic peptide
FIRNKPNGGTTEYAASVKD

SEQ ID NO: 54 - protein, 18 aa, Artificial Sequence
  Description of Artificial Sequence Synthetic peptide
SYISHCRGGVCYGGYFEF

SEQ ID NO: 55 - protein, 16 aa, Artificial Sequence
  Description of Artificial Sequence Synthetic peptide
RSSQSLKHSNGDTFLS

SEQ ID NO: 56 - protein, 7 aa, Artificial Sequence
  Description of Artificial Sequence Synthetic peptide
KVSNRDS

SEQ ID NO: 57 - protein, 9 aa, Artificial Sequence
  Description of Artificial Sequence Synthetic peptide
GQGTRTPPT

SEQ ID NO: 58 - protein, 6 aa, Artificial Sequence
  Description of Artificial Sequence Synthetic peptide
GGYGWG

SEQ ID NO: 59 - protein, 17 aa, Artificial Sequence
  Description of Artificial Sequence Synthetic peptide
SFYSSSGNTYYNPSLKS

SEQ ID NO: 60 - protein, 17 aa, Artificial Sequence
  Description of Artificial Sequence Synthetic peptide
DRLFSVVGMVYNNWFDV

SEQ ID NO: 61 - protein, 13 aa, Artificial Sequence
  Description of Artificial Sequence Synthetic peptide
TGSTSNIGGYDLH

SEQ ID NO: 62 - protein, 7 aa, Artificial Sequence
  Description of Artificial Sequence Synthetic peptide
DINKRPS

SEQ ID NO: 63 - protein, 11 aa, Artificial Sequence
  Description of Artificial Sequence Synthetic peptide
QSYDSSLNAQV

SEQ ID NO: 64 - protein, 69 aa, Artificial Sequence
  Description of Artificial Sequence Synthetic polypeptide
TTTPAPRPPTPAPTIASQPLSLRPEACRPAAGGAVHTRGLDFACDIYIWAPLAGTCGVLLLSLVITLYC

SEQ ID NO: 65 - protein, 54 aa, Artificial Sequence
  Description of Artificial Sequence Synthetic polypeptide
PSPADLSPGASSVTPPAPAREPGHSPQIISFFLALTSTALLFLLFFLTLRFSVV

SEQ ID NO: 66 - protein, 54 aa, Artificial Sequence; identical to SEQ ID NO: 65
  Description of Artificial Sequence Synthetic polypeptide

SEQ ID NO: 67 - protein, 15 aa, Artificial Sequence
  Description of Artificial Sequence Synthetic peptide
GGGGSGGGGSGGGGS

SEQ ID NO: 68 - protein, 10 aa, Artificial Sequence
  Description of Artificial Sequence Synthetic peptide
GGGGSGGGGS

(19) United States
(12) Patent Application Publication
(10) Pub. No.: US 2024/0093158 A1
(43) Pub. Date: Mar. 21, 2024

(54) ONCOLYTIC VIRUSES THAT EXPRESS MULTI-SPECIFIC IMMUNE CELL ENGAGERS

(71) Applicant: Arizona Board of Regents on Behalf of Arizona State University, Scottsdale, AZ (US)

(72) Inventors: Douglas Grant McFADDEN, Tempe, AZ (US); Lino TORRES-DOMINGUEZ, Tempe, AZ (US); Nancy VILLA, Tempe, AZ (US); Mohammed Masmudur RAHMAN, Chandler, AZ (US)

(21) Appl. No.: 17/767,856

(22) PCT Filed: Oct. 9, 2020
(86) PCT No.: PCT/US2020/055073

§ 371 Date: Apr. 8, 2022

Related U.S. Application Data
(60) Provisional application No. 62/913,655, filed on Oct. 10, 2019.

(51) Int. Cl. C12N 7/00; A61K 35/15; A61K 35/768; A61P 35/00; C07K 16/28

(52) U.S. Cl. CPC C12N 7/00; A61K 35/15; A61K 35/768; A61P 35/00; C07K 16/2809; C07K 16/283; C07K 16/2896; C07K 2317/21; C07K 2317/622; C12N 2710/24032

(57) ABSTRACT

The disclosure provides Myxoma viruses that express one or more multi-specific immune cell engagers, such as BiKE, BiTE and/or MiTE and their use in inhibiting and/or treating a hematological cancer in a subject. The disclosure also provides a leukocyte having a Myxoma virus that expresses one or more multi-specific immune cell engagers, and the use of the leukocyte for inhibiting and/or treating a hematological cancer in a subject.

### CROSS REFERENCE

This application claims the benefit of U.S. Provisional Patent Application No. 62/913,655, filed Oct. 10, 2019, which is incorporated herein by reference in its entirety.

### TECHNICAL FIELD

This disclosure relates to myxoma viruses and their uses for treatment of cancers, for example, treatment of hematological cancers with a myxoma virus that expresses one or more multi-specific immune cell engagers.

### BACKGROUND

Current treatments used to treat various types of cancer tend to work by poisoning or killing the cancerous cell. Unfortunately, treatments that are toxic to cancer cells typically tend to be toxic to healthy cells as well. Moreover, effective treatments for cancer remain elusive. Current mainstream therapies such as chemotherapy and radiotherapy can have a narrow therapeutic window (e.g., a concentration high enough to achieve efficacy but low enough to avoid toxicity). These types of therapies are considered blunt tools that have limited applicability due to the varying types of tumor cells and the limited window in which these treatments can be administered.

### SUMMARY

Disclosed herein, in some aspects, is a myxoma virus (MYXV) comprising a transgene that encodes a multi-specific immune cell engager.

In some embodiments, the multi-specific immune cell engager comprises a Bi-specific Natural Killer and Neutrophil engager (BiKE), a Bi-specific T Cell Engager (BiTE), or a membrane-integrated T cell engager (MiTE). In some embodiments, the multi-specific immune cell engager binds to an antigen present on a hematologic cancer cell. In some embodiments, the hematologic cancer cell is a myeloma cell, a leukemia cell, or a lymphoma cell. In some embodiments, the BiKE binds to an antigen present on a natural killer cell or a neutrophil. In some embodiments, the BiTE binds to an antigen present on a T cell. In some embodiments, the MiTE binds to an antigen present on a T cell. In some embodiments, the BiKE binds to CD16 or CD138. In some embodiments, the BiKE binds to CD16 and CD138. In some embodiments, the BiTE binds to CD3 or CD138. In some embodiments, the BiTE binds to CD3 and CD138. In some embodiments, the MiTE binds to CD3 or CD138. In some embodiments, MiTE binds to CD3 and CD138. In some embodiments, the multi-specific immune cell engager comprises one or more single chain variable fragments (scFvs) derived from an anti-human CD antibody. In some embodiments, the multi-specific immune cell engager comprises one or more humanized single chain variable fragments (scFvs). In some embodiments, the BiKE comprises a sequence that is at least 70%, at least 80%, at least 85%, at least 90%, at least 95%, at least 97%, at least 98%, at least 99%, or 100% identical to any one of SEQ ID NOs: 4-21. In some embodiments, the BiTE comprises a sequence that is at least 70%, at least 80%, at least 85%, at least 90%, at least 95%, at least 97%, at least 98%, at least 99%, or 100% identical to any one of SEQ ID NOs: 6, 7, 10-15, or 32-39. In some embodiments, the MiTE comprises a sequence that is at least 70%, at least 80%, at least 85%, at least 90%, at least 95%, at least 97%, at least 98%, at least 99%, or 100% identical to any one of SEQ ID NOs: 6, 7, 10-15, or 32-39. In some embodiments, the transgene is located between the M135 gene and M136 gene of the genome of the MYXV. In some embodiments, the MYXV further comprises a reporter gene. In some embodiments, the reporter gene encodes a fluorescent protein. In some embodiments, the reporter gene encodes a luminescent substrate or an enzyme. In some embodiments, the MYXV further comprises a mutation in the genome of the MYXV. In some embodiments, the mutation is present in one or more genes selected from the group consisting of M001R, M002R, M003.1R, M003.2R, M004.1R, M004R, M005R, M006R, M007R, M008.1R, M008R, M009L, M013, M036L, M063L, M11L, M128L, M131R, M135R, M136R, M141R, M148R, M151R, M152R, M153R, M154L, M156R, M-T2, M-T4, M-T5, M-T7, and SOD. In some embodiments, the mutation is a deletion. In some embodiments, the deletion deletes at least a portion of M135R. In some embodiments, the MYXV is present in a composition that comprises the MYXV and a pharmaceutically acceptable carrier. In some embodiments, the MYXV or the composition is administered to a subject in need thereof in a method of treating a hematological cancer. In some embodiments, the subject is a human. In some embodiments, the MYXV is capable of infecting cells that have a deficient innate anti-viral response. In some embodiments, the MYXV is capable of infecting cancer cells. In some embodiments, the hematological cancer is a myeloma, multiple myeloma, leukemia, or lymphoma. In some embodiments, the MYXV is administered to the subject with a leukocyte in a method for treating cancer, wherein the leukocyte comprises or is associated with the MYXV. In some embodiments, the method further comprises adsorbing the MYXV ex vivo onto a surface of the leukocyte. In some embodiments, the adsorbing the MYXV onto the surface of the leukocyte comprises exposing the leukocyte to the myxoma virus under conditions that permit binding of the myxoma virus to the surface of the leukocyte. In some embodiments, the adsorbing comprises exposing the leukocyte to the MYXV for at least five minutes. In some embodiments, adsorbing comprises exposing the leukocyte to the MYXV for about one hour. In some embodiments, the adsorbing comprises exposing the leukocyte to the MYXV at a multiplicity of infection (MOI) of between about 0.001 and 1000. In some embodiments, the adsorbing comprises exposing the leukocyte to the MYXV at a multiplicity of infection (MOI) of between about 0.1 and 10. In some embodiments, the leukocyte is obtained from peripheral blood. In some embodiments, the leukocyte is obtained from bone marrow. In some embodiments, the leukocyte is a peripheral blood mononuclear cell. In some embodiments, the leukocyte is obtained from the subject's tissue. In some embodiments, the leukocyte is obtained from a donor's tissue that is HLA-matched, HLA-mismatched, haploidentical, or a combination thereof relative to the subject. In some embodiments, the leukocyte is formulated in a pharmaceutical composition. In some embodiments, the leukocyte is administered systemically. In some embodiments, the leukocyte is administered parenterally. In some embodiments, the leukocyte is administered intravenously.

Some embodiments relate to a myxoma virus (MYXV) comprising a transgene that encodes a multi-specific immune cell engager.

Some embodiments relate to a composition comprising the myxoma virus described herein and a pharmaceutically acceptable carrier.

Some embodiments relate to a method of treating a hematological cancer in a subject in need thereof, comprising administering to the subject the myxoma virus described herein.

Some embodiments relate to a method of treating a hematological cancer in a subject in need thereof, comprising administering to the subject a leukocyte, wherein the leukocyte comprises the myxoma virus described herein.

### BRIEF DESCRIPTION OF THE DRAWINGS

The novel features of certain embodiments of the disclosure are set forth with particularity in the appended claims. A better understanding of the features and advantages of the present disclosure will be obtained by reference to the following detailed description that sets forth illustrative embodiments, in which the principles of the invention are utilized, and the accompanying drawings of which:

FIGS. 1A-1F show the construction of a MYXV-BiKE. FIG. 1A shows a scheme of the structure of the human CD138 targeted BiKE. FIG. 1B is a schematic diagram of the MYXV genome and the insertion site of the cassettes expressing BiKE and eGFP, both transgenes expressed under a poxvirus synthetic early/late promoter (sE/L). FIG. 1C shows a PCR analysis of genomic viral DNA from MYXV-BiKE clones using oligonucleotide primers to confirm presence of the BiKE cassette (panels 1-4) and FIG. 1D proper insertion (Intergenic region M135-M136). Lane 1 is DNA from MYXV-Lau, Lanes 2-4: MYXV-BiKE clones, M represents known size DNA ladder. FIG. 1E shows a Western blot analysis of cell lysates and supernatants from MYXV-BiKE infected RK13 cells 24 hours post-infection. FIG. 1F shows a single-step growth analysis of recombinant MYXV-BIKE vs MYXV-GFP.

FIGS. 2A-2C show MYXV-huBiKE-GFP productively infects and induces killing of cancer cells in in a blood sample taken from a multiple myeloma patient. FIG. 2A shows infection (GFP+), viability (Near IR−), apoptosis (Annexin V+) and cell killing (Near IR+) of multiple myeloma (MM) cells (CD138⁺) of a mock-treated (i.e., without adding MYXV) sample. FIG. 2B shows MYXV-huBiKE-GFP infection of CD138⁺ at three different MOI's. FIG. 2C shows apoptosis and cell death in CD138⁺ cells induced by MYXV-huBiKE-GFP.

FIGS. 3A and 3B show killing of uninfected multiple myeloma (MM) cells (i.e., GFP−) from a primary human sample from patient #3 after treatment with MYXV-huBiKE-GFP. FIG. 3A shows viability (Near IR−), apoptosis (Annexin V+) and cell killing (Near IR+) of uninfected MM cells (i.e., CD138⁺GFP⁻) in mock-treated (i.e., without adding MYXV) sample, after 24 hours. The arrow indicates gating on GFP− cells. FIG. 3B shows the percentages of apoptosis and cell death of uninfected MM cells that are CD138⁺GFP⁻ 24-hours post-infection.

FIGS. 4A-4D show MYXV-huBiKE-GFP productively infects and induces killing of multiple myeloma (MM) cells from a primary human sample from patient #4. FIG. 4A shows viability (Near IR−), infection (GFP+), apoptosis (Annexin V+), and cell killing (Near IR+) of MM cells (CD138*) after 24 hours of mock-treatment (i.e., without adding MYXV). FIG. 4B shows MYXV-huBiKE-GFP infection of CD138⁺ at three different MOI's. FIG. 4C shows apoptosis and cell death in CD138⁺ cells induced by MYXV-huBiKE-GFP. FIG. 4D shows fluorescence micrographs after 24-hours post-infection.

FIGS. 5A and 5B show killing of uninfected multiple myeloma (MM) cells (i.e., GFP−) from primary human sample from patient #4 after treatment with MYXV-huBiKE-GFP. FIG. 5A shows viability (Near IR−), apoptosis (Annexin V+), and cell killing (Near IR+) of uninfected MM cells (i.e., CD138⁺GFP⁻) in mock-treated (i.e., without adding MYXV) sample, after 24 hours. The arrow indicates gating on GFP− (uninfected). FIG. 5B shows the percentages of apoptosis and cell death in uninfected CD138⁺GFP⁻ cells at 24-hours post-treatment with virus.

FIG. 6 demonstrates that BiKE bound to human MM and NK cells, while binding was not detected for control MM cells or NK cells (incubated with supernatants harvested from mock-infected cells, or cells infected with a MYXV that lacks BiKE).

FIG. 7 demonstrates that the BiKE antibodies were able to induce NK-cell-mediated killing of MM cells, and that killing was dependent on the MOI of the source supernatant culture.

FIGS. 8A-D demonstrate the susceptibility of human hematologic cancer cells to MYXV-BiKE Infection was evaluated at 24 and 48 hpi by fluorescence microscopy. FIG. 8A and FIG. 8B demonstrate infection of THP-1 cells at 24 and 48 hours post-infection, respectively. FIG. 8C and FIG. 8D demonstrate infection of U266 cells at 24 and 48 hours post-infection, respectively.

FIG. 9 demonstrates killing of THP-1 cells by MYXV-BiKE, evaluated by flow cytometry.

FIG. 10 demonstrates killing of U266 cells by MYXV-BiKE, evaluated by flow cytometry.

FIG. 11 demonstrates killing of primary human multiple myeloma cells by MYXV-BiKE, evaluated by flow cytometry.

FIG. 12 provides a map for a plasmid that can be used to generate a MYXV of the disclosure that expresses a multi-specific immune cell engager.

FIG. 13 provides a map for a plasmid that can be used to generate a MYXV of the disclosure that expresses a multi-specific immune cell engager and comprises a gene disruption in the MYXV genome.

FIGS. 14A-14C show the BOR-resistant VK12598 cell line is susceptible to MYXV. FIG. 14A shows binding of Venus+ MYXV to the VK12598 cell line. FIG. 14B shows productive infection of the VK12598 cell line via fluorescence microscopy. FIG. 14C shows productive infection of the VK12598 cell line via flow cytometry.

FIGS. 15A and 15B show MYXV binding and infection of the multi-drug resistant VK12653 cell line. FIG. 15A shows binding of Venus+ MYXV to the VK12653 cell line. FIG. 15B shows productive infection of the VK12653 cell line via fluorescence microscopy and flow cytometry.

FIGS. 16A-16C show ex vivo therapy with myxoma virus to treat pre-existing multiple myeloma cancer in auto-transplant recipients. FIG. 16A shows a Western Blot providing the M-spike of mice four weeks post implantation with VK12598 cells (top panel) and four experimental cohorts (bottom panel).

FIG. 16B shows the percentage of MM cells (CD138⁺B220⁻) in a representative mock-treated mouse with low M-spike (0.1) and the percentage of MM (CD138⁺B220⁻) in a representative bone marrow-recipient mouse with high M-spike (0.6). FIG. 16C shows the M-spike of a mouse treated with bone marrow that had been ex vivo treated with MYXV-M135KO-GFP, with no M-spike band detected on day 8, day 29, and day 37 post-transplant.

FIG. 17A shows the percent of THP-1 cells that were GFP positive at 24 and 48 hours post-infection with MYXV-WT-GFP, MYXV-M135KO-GFP, and MYXV-BiKE-GFP.

FIG. 17B shows the percent of U266 cells that were GFP positive at 24 and 48 hours post-infection with MYXV-WT-GFP, MYXV-M135KO-GFP, and MYXV-BiKE-GFP.

FIG. 18A illustrates the percent of infected U266 cells that were killed at 24 and 48 hours by MYXV-WT-GFP and MYXV-BiKE-GFP.

FIG. 18B illustrates the percent of uninfected U266 cells that were killed at 24 and 48 hours by MYXV-WT-GFP and MYXV-BiKE-GFP.

FIG. 19 provides the ratio of dead U266 cells to infected U266 cells for cultures infected with MYXV-WT-GFP or MYXV-BiKE-GFP.

FIG. 20 illustrates the proportion of primary CD138+ MM cells that were infected by MYXV-BiKE-GFP, MYXV-M135KO-GFP, or wild type MYXV-GFP at the indicated MOI.

FIG. 21 quantifies the proportion of intact cells in primary human BM samples from MM patients that were CD138+ after mock-infection or infection with MYXV-BiKE-GFP or wild type MYXV-GFP at an MOI of 10.

FIG. 22 shows the percent of CD138+ MM cells that were dead after 48 hours of co-incubation with NK cells or PBMCs, in the presence of BiKE (from the supernatant of MYXV-BiKE-GFP-infected Vero cells) or the absence of BiKE (cRPMI, complete media; or supernatant from wild type MYXV-GFP-infected Vero cells). Co-cultures were performed in triplicate, and p values were obtained for each infection based on flow cytometric analysis of the proportion of the U266 cell population that were dead according to near-IR LIVE/DEAD stain. Significance (*=p<0.05; **=p<0.01; ***=p<0.001) was determined using Holm-Sidak's t test for multiple comparisons.

FIG. 23A provides dot-plots that demonstrate infection of CD138+ MM cells after co-incubation with MYXV-GFP or MYXV-BiKE-adsorbed NK cells (top row) or NK-depleted PBMCs (−NK, bottom row).

FIG. 23B provides dot-plots that demonstrate killing of CD138+ MM cells after co-incubation with MYXV-GFP or MYXV-BiKE-adsorbed NK cells (top row) or NK-depleted PBMCs (−NK, bottom row).

### DETAILED DESCRIPTION

Aspects of this disclosure relate to oncolytic virus recombinant constructs expressing multi-specific immune cell engagers and their uses for treating cancer such as hematologic cancer. The oncolytic virus can be a Myxoma virus (MYXV or vMyx, used interchangeably herein), and the multi-specific immune cell engagers used in the construct can include a BiKE (Bi-specific Natural Killer and Neutrophil engager) transgene, a BiTE (Bi-specific T cell Engager), and a membrane-integrated T cell engager (MiTE). The MYXV described herein can be used to treat hematological cancers, including minimal residual disease (MRD) and drug-resistant MRD.

The MYXV described herein can be a more effective therapy to treat hematologic cancer such as relapsed Multiple Myeloma disease, and to reduce, essentially reduce, or eliminate refractory and drug-resistant MRD. Multiple Myeloma (MM) is a hematologic malignancy characterized by a clonal expansion of malignant plasma cells resulting in end organ damage, including lytic bone lesions, anemia, renal failure, or hypercalcemia (Hari P. Recent advances in understanding multiple myeloma. Hematol Oncol Stem Cell Ther. 2017; In press). The bone marrow (BM) tumor microenvironment of MM plays a key role supporting and sustaining the differentiation, migration, proliferation, survival, and drug resistance of malignant MM cells (Kawano Y, Moschetta M, Manier S, Glavey S, Görgün G T, Roccaro A M, et al. Targeting the bone marrow microenvironment in multiple myeloma. Immunol Rev. 2015; 263(1)). Autologous stem cell transplantation for transplant eligible patients, along with chemotherapy, is a standard treatment for MM (Landgren O, Lu S X, and Hultcrantz M. MRD Testing in Multiple Myeloma: The Main Future Driver for Modern Tailored Treatment. Semin Hematol. 2018; 55(1):44-50; Hoyos V, and I. B. The immunotherapy era of myeloma: monoclonal antibodies, vaccines, and adoptive T-cell therapies. Blood. 2016; 128(13):1679-87). However, a major hurdle of these therapies is the relapse of the disease due to neoplastic clones that can serve as a reservoir of therapy-resistant MM cells, resulting in minimal residual disease (MRD).

Despite improvement in outcomes, MM is still considered incurable for most patients, and poor survival rates are observed in those patients with high-risk features (Bustoros M, Mouhieddine T H, Detappe A, and IM. G. Established and Novel Prognostic Biomarkers in Multiple Myeloma. Am Soc Clin Oncol Educ Book. 2017; 37:548-60). Oncolytic viruses such as MYXV are mammalian viruses that can be designed and/or selected for their ability to selectively infect and kill transformed cancer cells, and for their ability to activate the host immune system. The MYXV described herein utilizes a multi-specific immune cell engager, and can work in combination with the host immune systems to target cancer cells. Therefore, the Myxoma virus described herein can help reduce or eliminate the refractory and drug-resistant minimal residual disease and can be more effective to treat relapsed MM disease.

### Definitions

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCR Publishers, Inc., 1995 (ISBN 1-56081-569-8).

Unless otherwise explained, all technical and scientific terms used herein have the same meaning as commonly understood by one of ordinary skill in the art to which this disclosure belongs. Although methods and materials similar or equivalent to those described herein can be used in the practice or testing of this disclosure, suitable methods and materials are described below. In addition, the materials, methods, and examples are illustrative only and not intended to be limiting.

The following explanations of terms and methods are provided to better describe the present compounds, compositions, and methods, and to guide those of ordinary skill in the art in the practice of the present disclosure. It is also to be understood that the terminology used in the disclosure is for the purpose of describing particular embodiments and examples only and is not intended to be limited.

As used herein, the singular forms “a,” “an,” and “the” are intended to include the plural forms as well, unless the context clearly indicates otherwise.

As used herein, the term “and/or” refers to and encompasses any and all possible combinations of one or more of the associated listed items, as well as the lack of combinations when interpreted in the alternative (“or”).

As used herein, “one or more” or at least one can mean one, two, three, four, five, six, seven, eight, nine, ten or more, up to any number.

As used herein, the term “comprises” or “comprising” mean “includes.” Hence “comprising A or B” means including A, B, or A and B. “Comprise” and variations of the term, such as “comprising”, “comprises” and “comprised”, as used herein, mean that various additional components or steps can be conjointly employed.

An “effective amount” or “therapeutically effective amount” refers to an amount of a compound or composition of this disclosure that is sufficient to produce a desired effect, which can be a therapeutic and/or beneficial effect. In this example, the effective amount can vary with the age, general condition of the subject, the severity of the condition being treated, the particular agent administered, the duration of the treatment, the nature of any concurrent treatment, the pharmaceutically acceptable carrier used, and like factors within the knowledge and expertise of skilled workers. As appropriate, an effective amount or therapeutically effective amount in any individual case can be determined by reference to the pertinent texts and literature and/or by experimentation. (See, for example, Remington, The Science and Practice of Pharmacy (latest edition)).

As used herein, the term “subject” and “patient” are used interchangeably and refer to both human and nonhuman animals. The term “nonhuman animals” of the disclosure includes all vertebrates, e.g., mammals and non-mammals, such as nonhuman primates, sheep, dog, cat, horse, cow, rodents (e.g., mice, rats, etc.) and the like. A subject can be a human. A subject can be a human patient. In some embodiments, the subject of this disclosure is a human subject.

The term “a cell” as used herein includes a single cell as well as a plurality or population of cells. Administering or exposing an agent to a cell can include in vitro, ex vivo, and in vivo administering or exposing.

A “subject in need thereof” or “a subject in need of” is a subject known to have, or is suspected of having a cancer, such as a hematological cancer.

As used herein, the term “cancer” refers to a malignant neoplasm, for example, a neoplasm that has undergone characteristic anaplasia with loss of differentiation, increased rate of growth, invasion of surrounding tissue, and is capable of metastasis.

Residual cancer is cancer that remains in a subject after any form of treatment given to the subject to reduce or eradicate cancer. Metastatic cancer is a cancer at one or more sites in the body, e.g., a second site, other than the site of origin of the original (primary) cancer from which the metastatic cancer is derived. Local recurrence is reoccurrence of the cancer at or near the same site (such as in the same tissue) as the original cancer. Hematologic cancer is a cancer that affects the blood, bone marrow, and/or lymphatic system.

Non-limiting examples of hematologic cancers include leukemia, lymphoma, and myeloma, such as: multiple myeloma (MM); active multiple myeloma; smoldering multiple myeloma; plasmacytoma; solitary plasmacytoma of the bone; extramedullary plasmacytoma; light chain myeloma; non-secretory myeloma; immunoglobulin G (IgG) myeloma; immunoglobulin A (IgA) myeloma; immunoglobulin M (IgM) myeloma; immunoglobulin D (IgD) myeloma; immunoglobulin E (IgE) myeloma; hyperdiploid multiple myeloma; non-hyperdiploid multiple myeloma; Hodgkin lymphoma; non-Hodgkin lymphoma; acute lymphoblastic leukemia; acute myeloid leukemia; essential thrombocythemia; polycythemia vera; primary myelofibrosis; systemic mastocytosis; chronic myeloid leukemia; chronic neutrophilic leukemia; chronic eosinophilic leukemia; refractory anemia with ringed sideroblasts; refractory cytopenia with multilineage dysplasia; refractory anemia with excess blasts type 1; refractory anemia with excess blasts type 2; myelodysplastic syndrome (MDS) with isolated del (5q); MDS unclassifiable; chronic myelomonocytic leukemia (CML); atypical chronic myeloid leukemia; juvenile myelomonocytic leukemia; myeloproliferative/myelodysplastic syndromes-unclassifiable; B lymphoblastic leukemia/lymphoma; T lymphoblastic leukemia/lymphoma; diffuse large B-cell lymphoma; primary central nervous system lymphoma; primary mediastinal B-cell lymphoma; Burkitt lymphoma/leukemia; follicular lymphoma; chronic lymphocytic leukemia (CLL)/small lymphocytic lymphoma; B-cell prolymphocytic leukemia; lymphoplasmacytic lymphoma/Waldenstrom macroglobulinemia; Mantle cell lymphoma; marginal zone lymphomas; post-transplant lymphoproliferative disorders; HIV-associated lymphomas; primary effusion lymphoma; intravascular large B-cell lymphoma; primary cutaneous B-cell lymphoma; hairy cell leukemia; monoclonal gammopathy of unknown significance; Anaplastic large cell lymphoma, Angioimmunoblastic T-cell lymphoma, Hepatosplenic T-cell lymphoma, B-cell lymphoma, reticuloendotheliosis, reticulosis, Mucosa-associated lymphatic tissue lymphoma, B-cell chronic lymphocytic leukemia, Waldenstrom's macroglobulinemia, Lymphomatoid granulomatosis, Nodular lymphocyte predominant Hodgkin's lymphoma, plasma cell leukemia, Acute erythraemia and erythroleukaemia, Acute erythremic myelosis, Acute erythroid leukemia, Heilmeyer-Schoner disease, Acute megakaryoblastic leukemia, Mast cell leukemia, Panmyelosis, Acute panmyelosis with myelofibrosis, Lymphosarcoma cell leukemia, Stem cell leukemia, Chronic leukaemia of unspecified cell type, Subacute leukaemia of unspecified cell type, Accelerated phase chronic myelogenous leukemia, Acute promyelocytic leukemia, Acute basophilic leukemia, Acute eosinophilic leukemia, Acute monocytic leukemia, Acute myeloblastic leukemia with maturation, Acute myeloid dendritic cell leukemia, Adult T-cell leukemia/lymphoma, Aggressive NK-cell leukemia, B-cell chronic lymphocytic leukemia, B-cell leukemia, Chronic myelogenous leukemia, Chronic idiopathic myelofibrosis, Kahler's disease, Myelomatosis, Solitary myeloma, Plasma cell leukemia, Angiocentric immunoproliferative lesion, Lymphoid granulomatosis, Angioimmunoblastic lymphadenopathy, T-gamma lymphoproliferative disease, Waldenstrom's macroglobulinaemia, Alpha heavy chain disease, Gamma heavy chain disease, and Franklin's disease. In some embodiments, the hematological cancer is multiple myeloma.

As used herein, the term “chemotherapeutic agent” refers to any chemical agent with therapeutic usefulness in the treatment of diseases characterized by abnormal cell growth. Such diseases can include tumors, neoplasms, and cancer, as well as diseases characterized by hyperplastic growth such as psoriasis. In some embodiments, a chemotherapeutic agent is an agent of use in treating cancer, such as an anti-neoplastic agent. In some embodiments, a chemotherapeutic agent is a radioactive compound. One of skill in the art can readily identify a chemotherapeutic agent of use (see for example, Slapak and Kufe, Principles of Cancer Therapy, Chapter 86 in Harrison's Principles of Internal Medicine, 14th edition; Perry et al., Chemotherapy, Ch. 17 in Abeloff, Clinical Oncology 2nd ed., 2000 Churchill Livingstone, Inc; Baltzer and Berkery. (eds): Oncology Pocket Guide to Chemotherapy, 2nd ed. St. Louis, Mosby-Year Book, 1995; Fischer Knobf, and Durivage (eds): The Cancer Chemotherapy Handbook, 4th ed. St. Louis, Mosby-Year Book, 1993). Combination therapy is the administration of more than one agent to treat cancer. For example, a myxoma virus expressing one or more multi-specific immune cell engagers can be administered, and one or more chemotherapeutic agents can be administered, simultaneously or separated in time in any order.

“Treat,” “treatment,” or “treating,” as used herein refers to administering a pharmaceutical composition to a patient suffering from a disease or condition. As used herein, the term “inhibiting or treating a disease,” such as cancer, refers to delaying or inhibiting the development or progression of a disease or condition. “Treatment” refers to a therapeutic intervention that ameliorates a sign or symptom of a disease or pathological condition after it has begun to develop. The term “ameliorating,” with reference to a disease or pathological condition, refers to any observable beneficial effect of the treatment. The beneficial effect can be evidenced, for example, by a delayed onset of clinical symptoms of the disease in a susceptible subject, a reduction in severity of some or all clinical symptoms of the disease, a slower progression of the disease, such a metastasis, an improvement in the overall health or well-being of the subject, or by other parameters well known in the art that are specific to the particular disease. A “prophylactic” treatment is a treatment administered to a subject who does not exhibit signs of a disease or exhibits only early signs for the purpose of decreasing the risk of developing pathology, for example metastatic cancer.

As used herein the “pharmaceutically acceptable carriers” useful in conjunction with therapeutic compounds disclosed herein can be conventional. Remington's Pharmaceutical Sciences, by E. W. Martin, Mack Publishing Co., Easton, Pa., 19th Edition (1995), describes compositions and formulations suitable for pharmaceutical delivery of therapeutic agents.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example sodium acetate or sorbitan monolaurate.

As used herein, the terms “pharmaceutical” and “therapeutic agent” refer to a chemical compound or a composition capable of inducing a desired therapeutic or prophylactic effect when properly administered to a subject or a cell.

The term “replication-competent” as used herein refers to a virus, such as a myxoma virus, that is capable of infecting and replicating within a particular host cell, such as a human blood cell (e.g., a hematologic cancer cell, a multiple myeloma cell, or a peripheral blood mononuclear cell).

The term “immunomodulatory transgene” refers to a genetic sequence that can be introduced into a virus genome and encodes a product that can affect the function of the immune system, for example, that affects inflammation, innate or adaptive immune signaling, innate or adaptive immune cell activation (e.g., target cell killing, production of cytokines, chemokines, or other inflammatory mediators), innate or adaptive immune cell homing (e.g., chemotaxis, extravasation, and/or accumulation at a site), innate or adaptive immune cell proliferation, innate or adaptive immune cell differentiation, antibody production, or a combination thereof. Examples of immunomodulatory transgenes include, but are not limited to, BiTE, BiKE, and MiTE.

### Myxoma Virus

Myxoma virus (MYXV) is potentially well suited as a therapeutic virus against blood cancers, like multiple myeloma (MM), because of its unique biology. MYXV is a member of the poxviridae family and the leporipoxvirus genus (Chan W M, Rahman M M, and McFadden G. Oncolytic myxoma virus: the path to clinic. Vaccine. 2013; 31(39):4252-8, Chan W M, and McFadden G. Oncolytic Poxviruses. Annu Rev Virol. 2014; 1(1):119-41). Both MYXV and vMyx refer to Myxoma virus as described herein.

MYXV is a novel oncolytic virus that can target a variety of human and murine cancers, for example, both primary cancers and established cell lines (Stanford M M, and McFadden G. Myxoma virus and oncolytic virotherapy: a new biologic weapon in the war against cancer. Expert Opin Biol Ther. 2007; 7(9):1415-11425; Wang G, Barrett J W, Stanford M, Werden S J, Johnston J B, Gao X, et al. Infection of human cancer cells with myxoma virus requires Akt activation via interaction with a viral ankyrin-repeat host range factor. Proc Natl Acad Sci USA. 2006; 103(12):4640-5; Bartee E, Chan W M, Moreb J S, Cogle C R, and McFadden G. Selective purging of human multiple myeloma cells from autologous stem cell transplantation grafts using oncolytic myxoma virus. Biol Blood Marrow Transplant. 2012; 18(10):1540-51; Chan W M, Rahman M M, and McFadden G. Oncolytic myxoma virus: the path to clinic. Vaccine. 2013; 31(39):4252-8; Kim M, Madlambayan G J, Rahman M M, Smallwood S E, Meacham A M, Hosaka K, et al. Myxoma virus targets primary human leukemic stem and progenitor cells while sparing normal hematopoitic stem and progenitor cells. Leukemia. 2009; 32:2313-7; Villa N Y, Wasserfall C H, Meacham A M, Wise E, Chan W, Wingard J R, et al. Myxoma virus suppresses proliferation of activated T lymphocytes yet permits oncolytic virus transfer to cancer cells. Blood. 2015; 125(24):3778-88).

In nature, MYXV is rabbit-specific and generally does not cause infection or disease in humans, mice, or any other domestic animals. However, because of the nature of cancer pathway mutations associated with carcinogenesis, cancer cells from both mice and humans can exhibit a compromised ability to resist infection by some viruses, including MYXV (for example, compromised innate immune pathways) (Chan W M, and McFadden G. Oncolytic Poxviruses. Annu Rev Virol. 2014; 1(1):119-41, Sypula J, ‘Wang F, Ma Y, Bell J, and McFadden G. Myxoma virus tropism in human tumors. Gene Ther and Mol Biol. 2004; 8:103-14).

Provided herein, in some embodiments, are modified myxoma viruses (MYXV). The MYXV may be any virus that belongs to the Leporipoxvirus species of poxviruses that is replication-competent. The MYXV may be a wild-type strain of MYXV or it may be a genetically modified strain of MYXV. In some instances, the MYXV is Lausanne strain. In some instances, the MYXV is a South American MYXV strain that circulates in Sylvilagus brasiliensis. In some instances, the MYXV is a Californian MYXV strain that circulates in Sylvilagus bachmani. In some instances, the MYXV is 6918, an attenuated Spanish field strain that comprises modifications in genes M009L, M036L, M135R, and M148R (GenBank Accession number EU552530 which is hereby incorporated by reference as provided by GenBank on Aug. 27, 2019). In some instances, the MYXV is 6918VP60-T2 (GenBank Accession Number EU552531 which is hereby incorporated by reference as provided by GenBank on Aug. 27, 2019). In some instances, the MYXV is SG33, a strain comprising a genomic deletion that affects genes M151R, M152R, M153R, M154L, M156R, M008.1R, M008R, M007R, M006R, M005R, M004.1R, M004R, M003.2R, M003.1R, M002R, and M001R, (Collection Nationale de Cultures de Microorganismes (CNCM) Accession No. 1-1594). In some instances, the MYXV is a strain termed the Standard laboratory Strain (SLS).

In some instances, the MYXV genome comprises at least 70%, at least 75%, at least 80%, at least 85%, at least 90%, at least 95%, at least 96%, at least 97%, at least 98%, or at least 99%, such as between 95% and 98%, 95% and 99%, including 90%, 91%, 92%, 93%, 94%, 95%, 96%, 97%, 98%, or 99% nucleic acid sequence identity to a sequence disclosed in Cameron, et al., “The complete DNA sequence of Myxoma Virus,” Virology 264: 298-318 (1999), wherein is incorporated by reference in its entirety. In some cases, the MYXV comprises the sequence disclosed in Cameron, et al., “The complete DNA sequence of Myxoma Virus,” Virology 264: 298-318 (1999).

The large and genetically stable poxvirus genome allows for genetic manipulation, for example, generation of viruses with one or more deletions and/or introduction of one or more immunomodulatory transgenes, for example, one or more multi-specific immune cell engagers (Nayerossadat N, Maedeh T, and Ali P A. Viral and nonviral delivery systems for gene delivery. Adv Biomed Res. 2012; 1:27).

Provided herein, in some embodiments, are myxoma viruses (MYXV) and modified MYXV. The MYXV may be any virus that belongs to the Leporipoxvirus species of pox viruses that is replication-competent. The MYXV may be a wild-type strain of MYXV or it may be a genetically modified strain of MYXV.

The Myxoma virus genome can be modified to express one or more multi-specific immune cell engagers (e.g., BiKE, BiTE, and/or MiTE) using molecular biology techniques described herein and/or known to a skilled person, and described for example in Sambrook et al. ((2001) Molecular Cloning: a Laboratory Manual, 3rd ed., Cold Spring Harbour Laboratory Press). A skilled person will be able to determine which portions of the Myxoma viral genome can be deleted such that the virus is still capable of productive infection, for example, to provide a replication competent virus. For example, non-essential regions of the viral genome that can be deleted can be deduced from comparing the published viral genome sequence with the genomes of other well-characterized viruses (see for example C. Cameron, S. Hota-Mitchell, L. Chen, J. Barrett, J.-X. Cao, C. Macaulay, D. Willer, D. Evans, and G. McFadden, Virology (1999) 264: 298-318)).

In some embodiments, the disclosed MYXV recombinant construct is an oncolytic viral candidate to treat relapsed/refractory primary human hematologic malignancies such as multiple myeloma (MM) and to target and reduce or eliminate minimal residual disease (MRD). In some embodiments, the MYXV comprises one or more transgenes.

In some embodiments, a MYXV of the disclosure comprises one or more gene modifications, deletions, and/or disruptions in the MYXV genome. For example, a MYXV of the disclosure can comprise one or more insertions, deletions, or substitutions within or adjacent to one or more genes in the genome. An insertion, deletion or modification can comprise a gene knockout (for example, deletion of one or more nucleotides that thereby reduces or eliminates functionality of the product encoded by the gene, or insertion of one or more nucleotides that thereby disrupts expression and/or function of the product encoded by the gene). In some embodiments, an insertion, deletion, or modification does not comprise a gene knockout (for example, a sequence can be inserted at an intergenic locus between two genes, without disrupting expression of the two genes). A modification can be, for example, a transgene replacing a portion of a gene disclosed herein.

In some embodiments, a MYXV of the disclosure comprises one or more insertions, deletions, or substitutions within or adjacent to one or more genes associated with the ability of the virus to cause disease in a host animal. In some embodiments, a MYXV of the disclosure comprises one or more insertions, deletions, or substitutions within or adjacent to one or more genes associated with host cell tropism. In some embodiments, a MYXV of the disclosure comprises one or more insertions, deletions, or substitutions within or adjacent to one or more genes associated with the ability of the virus to evade an innate immune response. In some embodiments, a MYXV of the disclosure comprises one or more insertions, deletions, or substitutions within or adjacent to one or more genes that modulate immune signaling in an infected cell (e.g., cytokine receptor signaling). In some embodiments, a MYXV of the disclosure comprises one or more insertions, deletions, or substitutions within or adjacent to one or more genes that modulate a cell death pathway in an infected cell (e.g., a gene that codes for a product that promotes or inhibits apoptosis, such as M011L gene accession number GQ398535).

In some embodiments, a MYXV of the disclosure comprises one or more insertions, deletions, or substitutions within or adjacent to one or more genes that modulates viral replication in a cancer cell (e.g., increases or decreases the rate of viral replication in a cancer cell).

In some embodiments, the one or more genes associated with the ability of the virus to cause disease in a host animal, associated with host cell tropism, associated with the ability of the virus to evade an innate immune response, that can modulate immune signaling in an infected cell, that can modulate a cell death pathway in an infected cell, that can modulate viral replication in a cancer cell, or a combination thereof, comprise any one or more of M001R, M002R, M003.1R, M003.2R, M004.1R, M004R, M005R, M006R, M007R, M008.1R, M008R, M009L, M013, M036L, M063L, M011L, M128L, M131R, M135R, M136R, M141R, M148R, M151R, M152R, M153R, M154L, M156R, M-T2, M-T4, M-T5, M-T7, and SOD.

In some embodiments, a MYXV of the disclosure comprises a modification of a MYXV gene. In some instances, the modification is a deletion that impairs the function of a protein encoded by the MYXV gene. In some cases, the modification is a partial deletion. For example, a partial deletion can be an at least 10%, at least 20%, at least 30%, at least 40%, at least 50%, at least 60%, at least 70%, at least 80%, at least 90%, or at least 95% deletion of the MYXV gene. In some embodiments, a partial deletion can be an at most 10%, at most 20%, at most 30%, at most 40%, at most 50%, at most 60%, at most 70%, at most 80%, at most 90%, or at most 95% deletion of the MYXV gene. In other cases, the modification is a full deletion of the MYXV gene (e.g., deletion of entire coding region, deletion of entire gene, etc.). In some embodiments, the modification is a replacement of the MYXV gene with one or more transgenes of the disclosure (e.g., multi-specific immune cell engagers, such as BiKE, BiTE, and/or MiTE).

In some embodiments, a MYXV of the disclosure comprises one or more insertions, deletions, or substitutions within or adjacent to one or more genes associated with host cell tropism (for example, rabbit cell tropism). In some embodiments, one or more genes associated with rabbit cell tropism comprises M11L, M063, M135R, M136R, M-T2, M-T4, M-T5, M-T7, or a combination thereof. In some instances, the one or more genes associated with rabbit cell tropism comprise M135R, M136R, or a combination thereof.

In some embodiments, a MYXV of the disclosure comprises a modification of the M135R gene. In some embodiments, the MYXV comprises a partial deletion or full deletion of M135R gene. A deletion or disruption of the M135R gene can, for example, attenuate the ability of a MYXV of the disclosure to cause disease in a host animal, without impairing the ability of the MYXV to exhibit an anti-cancer effect (e.g., infect and kill cancer cells).

In some instances, the modification is a deletion that impairs the function of a protein encoded by the M135R gene. In some cases, the modification is a partial deletion (e.g., an at least 10%, at least 20%, at least 30%, at least 40%, at least 50%, at least 60%, at least 70%, at least 80%, at least 90%, at least 95% deletion, at most 10%, at most 20%, at most 30%, at most 40%, at most 50%, at most 60%, at most 70%, at most 80%, at most 90%, at most 95%, about 10%, about 20%, about 30%, about 40%, about 50%, about 60%, about 70%, about 80%, about 90%, or about 95% deletion) of the M135R gene. In other cases, the modification is a full deletion of the M135R gene (e.g., deletion of entire coding region of M135 gene, deletion of entire M135 gene, etc.). In some embodiments, the deletion is a deletion of at least 1, at least 2, at least 3, at least 4, at least 5, at least 7, at least 10, at least 20, at least 30, at least 40, at least 50, at least 60, at least 70, at least 100, at least 200, or at least 300 nucleic acids. In some embodiments, the deletion disrupts a promoter (e.g., a promoter that drives expression of M135R in a wild type MYXV). In some embodiments, the deletion introduces a stop codon into the M135R gene sequence, for example, a premature stop codon that prevents expression of a full length M135R transcript and/or protein.

In some embodiments, the MYXV comprises a modification of M135R gene that impairs the function of M135R gene (e.g., insertion of a sequence that disrupts the expression and/or function of the M135R gene). In some embodiments, the insertion is an insertion of at least 1, at least 2, at least 3, at least 4, at least 5, at least 7, at least 10, at least 20, at least 30, at least 40, at least 50, at least 60, at least 70, at least 100, at least 200, at least 300, at least 400, at least 500, at least 600, at least 700, at least 800, at least 900, at least 1000, at least 1500, or at least 2000 nucleic acids. In some embodiments, the insertion alters the reading frame of the M135R gene sequence, thereby disrupting expression of the M135R transcript and/or protein.

In some instances, the mutation is a substitution, for example, a substitution that attenuates an activity or expression level of a protein encoded by the M135R gene. In some embodiments, at least 1, at least 2, at least 3, at least 4, at least 5, at least 7, at least 10, at least 20, at least 30 nucleic acids are substituted. In some embodiments, the substitution introduces a stop codon into the M135R gene sequence, for example, a premature stop codon that prevents expression of a full length M135R transcript and/or protein. In some embodiments, the substitution disrupts a promoter (e.g., a promoter that drives expression of M135R in a wild type MYXV).

In some embodiments, a modification or mutation disclosed herein attenuates the activity level of the M135R gene and/or protein by at least about 10%, at least about 20%, at least about 30%, at least about 40%, at least about 50%, at least about 60%, at least about 70%, at least about 80%, at least about 90%, at least about 95%, at least about 97%, or at least about 99% relative to a wild type MYXV, or a MYXV that encodes a functional wild type M135R.

In some embodiments, a modification or mutation disclosed herein attenuates the expression level of the M135R gene and/or protein by at least about 10%, at least about 20%, at least about 30%, at least about 40%, at least about 50%, at least about 60%, at least about 70%, at least about 80%, at least about 90%, at least about 95%, at least about 97%, or at least about 99% relative to a wild type MYXV, or a MYXV that encodes a functional wild type M135R.

In some embodiments, a MYXV disclosed herein has an activity level of the M135R protein that is attenuated by at least about 10%, at least about 20%, at least about 30%, at least about 40%, at least about 50%, at least about 60%, at least about 70%, at least about 80%, at least about 90%, at least about 95%, at least about 97%, or at least about 99% relative to a wild type MYXV, or a MYXV that encodes a functional wild type M135R.

In some embodiments, a MYXV disclosed herein has an expression level of the M135R gene and/or protein that is attenuated by at least about 10%, at least about 20%, at least about 30%, at least about 40%, at least about 50%, at least about 60%, at least about 70%, at least about 80%, at least about 90%, at least about 95%, at least about 97%, or at least about 99% relative to a wild type MYXV, or a MYXV that encodes a functional wild type M135R.

In some embodiments, a transgene of the disclosure replaces the M135R gene within the MYXV genome, for example, disrupts or replaces the M135R gene with one or more transgenes of the disclosure (e.g., a multi-specific immune cell engager, such as BiKE, BiTE, and/or MiTE). In some embodiments, a transgene of the disclosure replaces a portion of the M135R gene within the MYXV genome. In some embodiments, a transgene of the disclosure is inserted between M135R gene and M136R gene within the MYXV genome. In some embodiments, a transgene of the disclosure is inserted in the M135-136 locus. For MYXV, 136 as used herein can refer to M136 gene locus of the MYXV. In some embodiments, M136 refers to M136R of MYXV.

In some embodiments, a MYXV of the disclosure comprises a modification of the M153 gene. The M153 gene product is an E3-Ubiquitin ligase that may participate in the down regulation of diverse cellular receptors and proteins, for example, degradation of MHC Class I and CD4 in human cells. In some embodiments, a MYXV of the disclosure has an attenuated activity and/or expression level of M153 protein. In some embodiments, an attenuated activity and/or expression level of M153 protein can enhance presentation of immune epitopes, for example, MHC-dependent presentation of viral and/or cancer immune peptides. Enhanced presentation of immune epitopes by infected cancer cells can elicit stronger immune responses, including anti-cancer T cell responses, such as anti-cancer CD8+ T cell responses. In some embodiments, an attenuated activity and/or expression level of M153 protein increases direct antigen presentation from M153KO virus-infected tumor cells by MHC-I, and enhances immune activation mediated by the MYXV.

In some embodiments, the MYXV comprises a partial deletion or full deletion of M153 gene. In some instances, the modification is a deletion that impairs the function of a protein encoded by the M153 gene. In some cases, the modification is a partial deletion (e.g., at least 10%, at least 20%, at least 30%, at least 40%, at least 50%, at least 60%, at least 70%, at least 80%, at least 90%, at least 95% deletion, at most 10%, at most 20%, at most 30%, at most 40%, at most 50%, at most 60%, at most 70%, at most 80%, at most 90%, at most 95%, about 10%, about 20%, about 30%, about 40%, about 50%, about 60%, about 70%, about 80%, about 90%, or about 95% deletion) of the M153 gene. In some embodiments, the deletion is a deletion of at least 1, at least 2, at least 3, at least 4, at least 5, at least 7, at least 10, at least 20, at least 30, at least 40, at least 50, at least 60, at least 70, at least 100, at least 200, or at least 300 nucleic acids. In some embodiments, the deletion disrupts a promoter (e.g., a promoter that drives expression of M153 in a wild type MYXV). In some embodiments, the deletion introduces a stop codon into the M153 gene sequence, for example, a premature stop codon that prevents expression of a full length M153 transcript and/or protein.

In other cases, the modification is a full deletion of the M153 gene (e.g., deletion of entire coding region of M153 gene, deletion of entire M153 gene, etc.). In some embodiments, the MYXV comprises a modification of M153 gene that impairs the function of M153 gene (e.g., insertion of a sequence that disrupts the expression and/or function of the M153 gene). In some embodiments, the insertion is an insertion of at least 1, at least 2, at least 3, at least 4, at least 5, at least 7, at least 10, at least 20, at least 30, at least 40, at least 50, at least 60, at least 70, at least 100, at least 200, at least 300, at least 400, at least 500, at least 600, at least 700, at least 800, at least 900, at least 1000, at least 1500, or at least 2000 nucleic acids. In some embodiments, the insertion alters the reading frame of the M153 gene sequence, thereby disrupting expression of the M153 transcript and/or protein.

In some instances, the mutation is a substitution, for example, a substitution that attenuates an activity or expression level of a protein encoded by the M153 gene. In some embodiments, at least 1, at least 2, at least 3, at least 4, at least 5, at least 7, at least 10, at least 20, at least 30 nucleic acids are substituted. In some embodiments, the substitution introduces a stop codon into the M153 gene sequence, for example, a premature stop codon that prevents expression of a full length M153 transcript and/or protein. In some embodiments, the substitution disrupts a promoter (e.g., a promoter that drives expression of M153 in a wild type MYXV).

In some embodiments, a modification or mutation disclosed herein attenuates the activity level of the M153 gene and/or protein by at least about 10%, at least about 20%, at least about 30%, at least about 40%, at least about 50%, at least about 60%, at least about 70%, at least about 80%, at least about 90%, at least about 95%, at least about 97%, or at least about 99% relative to a wild type MYXV, or a MYXV that encodes a functional wild type M153.

In some embodiments, a modification or mutation disclosed herein attenuates the expression level of the M153 gene and/or protein by at least about 10%, at least about 20%, at least about 30%, at least about 40%, at least about 50%, at least about 60%, at least about 70%, at least about 80%, at least about 90%, at least about 95%, at least about 97%, or at least about 99% relative to a wild type MYXV, or a MYXV that encodes a functional wild type M153.

In some embodiments, a MYXV disclosed herein has an activity level of the M153 protein that is attenuated by at least about 10%, at least about 20%, at least about 30%, at least about 40%, at least about 50%, at least about 60%, at least about 70%, at least about 80%, at least about 90%, at least about 95%, at least about 97%, or at least about 99% relative to a wild type MYXV, or a MYXV that encodes a functional wild type M153.

In some embodiments, a MYXV disclosed herein has an expression level of the M153 gene and/or protein that is attenuated by at least about 10%, at least about 20%, at least about 30%, at least about 40%, at least about 50%, at least about 60%, at least about 70%, at least about 80%, at least about 90%, at least about 95%, at least about 97%, or at least about 99% relative to a wild type MYXV, or a MYXV that encodes a functional wild type M153.

In some embodiments, a transgene of the disclosure replaces the M153 gene within the MYXV genome, for example, disrupts or replaces the M153 gene with one or more multi-specific immune cell engagers of the disclosure such as BiKE, MiTE, and/or BiTE. In some embodiments, a transgene of the disclosure replaces a portion of the M153 gene within the MYXV genome (for example, replaces a portion of the M153 gene BiKE, MiTE, and/or BiTE).

### Transgenes

Provided herein, in some embodiments, are myxoma virus (MYXV) recombinant constructs comprising transgenes. In the context of cancer and the tumor microenvironment, a range of immunomodulatory factors can affect the interplay between cancer cells and the immune system. One or more immunomodulatory transgenes can be introduced into the MYXV genome, for example, to promote an immune response that more effectively treats or reduces a cancer. In some embodiments, one or more MYXV endogenous genes are ablated, and one or more immunomodulatory transgenes are introduced to the viral genome. In some embodiments, the transgene encodes a multi-specific immune cell engager, such as BiKE, BiTE, and/or MiTE.

Multi-specific immune cell engagers can comprise the ability to specifically bind at least one antigen or epitope. In some embodiments, a multi-specific immune cell of the disclosure can bind one, two, three, four, five, six, seven, eight, nine, ten, or more target antigens or epitopes. In some embodiments, a multi-specific immune cell of the disclosure can bind at least two, at least three, at least four, at least five, at least six, at least seven, at least eight, at least nine, at least ten, or more target antigens or epitopes.

In some embodiments, a membrane-integrated immune cell engager, such as a MiTE, binds to one target antigen or epitope. For example, a membrane-integrated immune cell engager of the disclosure can be expressed on the surface of cancer cells that are susceptible to infection by a MYXV of the disclosure, and the membrane-integrated immune cell engager can bind to an immune cell, such as a T cell, neutrophil, or NK cell.

In some embodiments, a multi-specific immune cell engager of the disclosure can bind to an antigen or epitope expressed by a cancer cell (e.g., cell of a hematologic cancer as disclosed herein). In some embodiments, a multi-specific immune cell engager of the disclosure can bind to an antigen or epitope expressed on the surface of a cancer cell. In some embodiments, the antigen or epitope is a wild-type antigen or epitope (for example, is not mutated). In some embodiments, the antigen or epitope is not a wild-type antigen or epitope (for example, a neoepitope that arises during cancerous mutations). In some embodiments, the antigen or epitope is an oncogene. In some embodiments, the antigen or epitope is a mutated tumor suppressor gene. Non-limiting examples of antigens or epitopes expressed by cancer cells that can be bound by multi-specific immune cell engagers of the disclosure include CD138, CD19, CD20, CD22, CD70, CD79a, CD79b, EpCAM, Her2, Her2/neu, EGFR, CEA, CD33, and MCSP. In some embodiments, a multi-specific immune cell engager of the disclosure binds CD138.

In some embodiments, a multi-specific immune cell engager of the disclosure can bind to an antigen or epitope expressed by an immune cell. In some embodiments, a multi-specific immune cell engager of the disclosure can bind to an antigen or epitope expressed on the surface of an immune cell. In some embodiments, a multi-specific immune cell engager of the disclosure binding to an antigen or epitope expressed by an immune cell can promote activation of a signaling pathway in the immune cell. In some embodiments, a multi-specific immune cell engager of the disclosure binding to an antigen or epitope expressed by an immune cell can promote activation of the immune cell. In some embodiments, a multi-specific immune cell engager of the disclosure binding to an antigen or epitope expressed by an immune cell can promote cytolytic killing by the immune cell (e.g., killing of a cancer cell). In some embodiments, a multi-specific immune cell engager of the disclosure binding to an antigen or epitope expressed by an immune cell can promote the production of pro-inflammatory cytokines by the immune cell. In some embodiments, a multi-specific immune cell engager of the disclosure binding to an antigen or epitope expressed by an immune cell can promote signaling via CD3.

Non-limiting examples of immune cell subsets that can be bound by multi-specific immune cell engagers of the disclosure include lymphocytes, T cells, CD4+ T cells, CD8+ T cells, alpha-beta T cells, gamma-delta T cells, T regulatory cells (Tregs), cytotoxic T lymphocytes, Th1 cells, Th2 cells, Th17 cells, Th9 cells, naïve T cells, memory T cells, effector T cells, effector-memory T cells (TEM), central memory T cells (TCM), resident memory T cells (TRM), follicular helper T cells (TFH), naïve T cells, Natural killer T cells (NKTs), tumor-infiltrating lymphocytes (TILs), Natural killer cells (NKs), Innate Lymphoid Cells (ILCs), ILC1 cells, ILC2 cells, ILC3 cells, lymphoid tissue inducer (LTi) cells, B cells, B1 cells, B1a cells, B1b cells, B2 cells, plasma cells, B regulatory cells, memory B cells, marginal zone B cells, follicular B cells, germinal center B cells, antigen presenting cells (APCs), monocytes, macrophages, M1 macrophages, M2 macrophages, tissue-associated macrophages, dendritic cells, plasmacytoid dendritic cells, neutrophils, mast cells, basophils, eosinophils, and combinations thereof. In some embodiments, a multi-specific immune cell engager of the disclosure binds T cells. In some embodiments, a multi-specific immune cell engager of the disclosure binds NK cells. In some embodiments, a multi-specific immune cell engager of the disclosure binds neutrophils.

Non-limiting examples of antigens expressed by immune cells that can be bound by multi-specific immune cell engagers of the disclosure include CD2, CD3, CD4, CD5, CD7, CD8, CD11b, CD13, CD15, CD16, CD25, CD32, CD33, CD27, CD28, CD40, CD56, CD69, CD80, CD83, CD86, CD94, CD122, CD127, CD134, MHC-II, CD195, CD282, CD284, CD314, CD336, CD337, KLRG1, and TIGIT. In some embodiments, a multi-specific immune cell engager of the disclosure binds CD3. In some embodiments, a multi-specific immune cell engager of the disclosure binds CD16.

In some embodiments, a MYXV of the disclosure comprises a BiKE (Bi-specific Natural Killer and Neutrophil engager) transgene. In some embodiments, a BiKE transgene comprises a sequence derived from one or more antibodies (e.g., one or more heavy chain variable domains, one or more light chain variable domains, one or more complementarity determining regions (CDRs), or a combination thereof). In some embodiments, a BiKE transgene comprises a sequence derived from one or more mammalian antibodies. In some embodiments, a BiKE transgene comprises a sequence derived from one or more mouse antibodies. In some embodiments, a BiKE transgene comprises a sequence derived from one or more humanized antibodies (huBiKE), such as a fully-human antibody. In some embodiments, a BiKE transgene encodes a product that is secreted. In some embodiments, a BiKE transgene encodes a product that localizes to the cell surface (e.g., comprises a transmembrane domain). In some embodiments, a BiKE gene comprises or encodes a sequence from any one or more of SEQ ID NOs: 6-21, as provided in Table 1. SEQ ID NOs: 6-7 provide the sequences of variable regions from an antibody specific for CD138. SEQ ID NOs: 8-9 provide the sequences of variable regions from an antibody specific for CD16. SEQ ID NOs: 10-15 provide the sequences of CDRs from antibodies specific for CD138, as identified by the method of Kabat. SEQ ID NOs: 16-21 provide the sequences of CDRs from antibodies specific for CD16, as identified by the method of Kabat.

In some embodiments, a BiKE comprises a sequence that comprises, consists essentially of, or consists of an amino acid sequence with at least about 70%, at least about 80%, at least about 90%, at least about 95%, at least about 97%, or at least about 99% sequence identity to any one of SEQ ID NOs: 6-21. In some embodiments, a MYXV of the disclosure encodes a BiKE that comprises, consists essentially of, or consists of an amino acid sequence that is any one of SEQ ID NOs: 6-21.

In some embodiments, a MYXV of the disclosure comprises a BiTE (Bi-specific T cell engager) transgene. In some embodiments, a BiTE transgene comprises a sequence derived from one or more antibodies (e.g., one or more heavy chain variable domains, one or more light chain variable domains, one or more complementarity determining regions (CDRs), or a combination thereof). In some embodiments, a BiTE transgene comprises a sequence derived from one or more mammalian antibodies. In some embodiments, a BiTE transgene comprises a sequence derived from one or more mouse antibodies. In some embodiments, a BiTE transgene comprises a sequence derived from one or more humanized antibodies (huBiTE), such as a fully-human antibody. In some embodiments, a BiTE transgene encodes a product that is secreted. In some embodiments, a BiTE transgene encodes a product that localizes to the cell surface (e.g., comprises a transmembrane domain).

In some embodiments, a BiTE gene comprises a sequence from any one or more of SEQ ID NOs: 6, 7, 10-15, 32, 33, or 34-63, as provided in Table 1. In some embodiments, a BiTE comprises a sequence that comprises, consists essentially of, or consists of an amino acid sequence with at least about 70%, at least about 80%, at least about 90%, at least about 95%, at least about 97%, or at least about 99% sequence identity to any one of SEQ ID NOs: 6, 7, 10-15, 32, 33, or 34-63. In some embodiments, a MYXV of the disclosure encodes a BiTE that comprises, consists essentially of, or consists of an amino acid sequence that is any one of SEQ ID NOs: 6, 7, 10-15, 32, 33, or 34-63.

SEQ ID NOs: 6-7 provide the sequences of variable regions from an antibody specific for CD138. SEQ ID NOs: 10-15 provide the sequences of CDRs from antibodies specific for CD138, as identified by the method of Kabat. SEQ ID NOs: 32-33 provide the sequences of variable regions from an antibody specific for CD3. SEQ ID NOs: 34-39 provide the sequences of CDRs from antibodies specific for CD3, as identified by the method of Kabat. SEQ ID NOs: 40-45 provide the sequences of variable regions from an antibody specific for CD80. SEQ ID NOs: 46-63 provide the sequences of CDRs from antibodies specific for CD80, as identified by the method of Kabat.

In some embodiments, a MYXV of the disclosure comprises a MiTE (membrane-integrated T cell engager) transgene. In some embodiments, a MiTE transgene encodes a product that localizes to the cell surface (e.g., comprises a transmembrane domain). In some embodiments, a MiTE transgene comprises a sequence derived from one or more antibodies (e.g., one or more heavy chain variable domains, one or more light chain variable domains, one or more complementarity determining regions (CDRs), or a combination thereof). In some embodiments, a MiTE transgene comprises a sequence derived from one or more mammalian antibodies. In some embodiments, a MiTE transgene comprises a sequence derived from one or more mouse antibodies. In some embodiments, a MiTE transgene comprises a sequence derived from one or more humanized antibodies (huMiTE), e.g., fully human antibodies.

In some embodiments, a MiTE gene comprises a sequence from any one or more of SEQ ID NOs: 6, 7, 10-15, 32, 33, or 34-63, as provided in Table 1. In some embodiments, a MiTE comprises a sequence that comprises, consists essentially of, or consists of an amino acid sequence with at least about 7000, at least about 80%, at least about 90%, at least about 95%, at least about 97%, or at least about 99% sequence identity to any one of SEQ ID NOs: 6, 7, 10-15, 32, 33, or 34-63. In some embodiments, a MYXV of the disclosure encodes a MiTE that comprises, consists essentially of, or consists of an amino acid sequence that is any one of SEQ TD NOs: 6, 7, 10-15, 32, 33, or 34-63.

SEQ ID NOs: 6-7 provide the sequences of variable regions from an antibody specific for CD138. SEQ ID NOs: 10-15 provide the sequences of CDRs from antibodies specific for CD138, as identified by the method of Kabat. SEQ ID NOs: 32-33 provide the sequences of variable regions from an antibody specific for CD3. SEQ ID NOs: 34-39 provide the sequences of CDRs from antibodies specific for CD3, as identified by the method of Kabat. SEQ ID NOs: 40-45 provide the sequences of variable regions from an antibody specific for CD80. SEQ ID NOs: 46-63 provide the sequences of CDRs from antibodies specific for CD80, as identified by the method of Kabat.

TABLE 1

| SEQ ID NO: | Description | Sequence |
|---|---|---|
| 6 | Anti-CD138 | SQVQLQQSGSELMMPGASVKISCKATGY |
| | VH | TFSNYWIEWVKQRPGHGLEWIGEILPGT |
| | | GRTIYNEKFKGKATFTADISSNTVQMQL |
| | | SSLTSEDSAVYYCARRDYYGNFYYAMDY |
| | | WGQGTSVTVSS |
| 7 | Anti-CD138 | DIQMTQSTSSLSASLGDRVTISCSASQG |
| | VL | INNYLNWYQQKPDGTVELLIYYTSTLQS |
| | | GVPSRFSGSGSGTDYSLTISNLEPEDIG |
| | | TYYCQQYSKLPRTFGGGTKLEIK |
| 8 | Anti-CD16 | QVTLKESGPGILQPSQTLSLTCSFSGFS |
| | VH | LRTSGMGVGWIRQPSGKGLEWLAHIWWD |
| | | DDKRYNPALKSRLTISKDTSSNQVFLKI |
| | | ASVDTADTATYYCAQINPAWFAYWGQGT |
| | | LVTVSA |
| 9 | Anti-CD16 | DTVLTQSPASLAVSLGQRATISCKASQS |
| | VL | VDFDGDSFMNWYQQKPGQPPKLLIYTTS |
| | | NLESGIPARFSASGSGTDFTLNIHPVEE |
| | | EDTATYYCQQSNEDPYTFGGGTKLEIK |
| 32 | Anti-CD3 | MQVQLLESGAELARPGASVKMSCKASGY |
| | VH | TFTRYTMHWVKQRPGQGLEWIGYINPSR |
| | | GYTNYNQKFKDKATLTTDKSSSTAYMQL |
| | | SSLTSEDSAVYYCAGYYDDHYCLDYWGQ |
| | | GTLVTVSS |
| 33 | Anti-CD3 | DIVMTQSPAIMSASPGEKVTMTCSASSS |
| | VL | VSYMNWYQQKSGTSPKRWIYDTSKLASG |
| | | VPAHFRGSGSGTSYSLTISGMEAEDAAT |
| | | YYCQQWSSNPFTFGSGTKLEIKR |
| 40 | Anti-CD80 | QVKLQQWGEGLLQPSETLSRTCVVSGGS |
| | VH-A | ISGYYYWTWIRQTPGRGLEWIGHIYGNG |
| | | ATTNYNPSLKSRVTISKDTSKNQFFLNL |
| | | NSVTDADTAVYYCARGPRPDCTTICYGG |
| | | WVDVWGPGDLVTVSS |
| 41 | Anti-CD80 | AYELTQPPSVSVSPGQTARITCGGDNSR |
| | VL-A | NEYVHWYQQKPARAPILVIYDDSDRPSG |
| | | IPERFSGSKSGNTATLTINGVEAGDEAD |
| | | YYCQVWDRASDHPVFGGGTRVTVL |
| 42 | Anti-CD80 | EVQLVESGGGLVQPGGSLRVSCAVSGFT |
| | VH-B | FSDHYMYWFRQAPGKGPEWVGFIRNKPN |
| | | GGTTEYAASVKDRFTISRDDSKSIAYLQ |
| | | MSSLKIEDTAVYYCTTSYISHCRGGVCY |
| | | GGYFEFWGQGALVTVSS |
| 43 | Anti-CD80 | EVVMTQSPLSLPITPGEPASISCRSSQS |
| | VL-B | LKHSNGDTFLSWYQQKPGQPPRLLIYKV |
| | | SNRDSGVPDRFSGSGAGTDFTLKISAVE |
| | | AEDVGVYFCGQGTRTPPTFGGGTKVEIK |
| 44 | Anti-CD80 | QVQLQESGPGLVKPSETLSLTCAVSGGS |
| | VH-C | ISGGYGWGWIRQPPGKGLEWIGSFYSSS |
| | | GNTYYNPSLKSQVTISTDTSKNQFSLKL |
| | | NSMTAADTAVYYCVRDRLFSVVGMVYNN |
| | | WFDVWGPGVLVTVSS |
| 45 | Anti-CD80 | ESVLTQPPSVSGAPGQKVTISCTGSTSN |
| | VL-C | IGGYDLHWYQQLPGTAPKLLIYDINKRP |
| | | SGISDRFSGSKSGTAASLAITGLQTEDE |
| | | ADYYCQSYDSSLNAQVFGGGTRLTVL |
| 10 | Anti-CD138 | NYWIE |
| | HCDR1 | |
| 11 | Anti-CD138 | EILPGTGRTIYNEKFKG |
| | HCDR2 | |
| 12 | Anti-CD138 | RDYYGNFYYAMDY |
| | HCDR3 | |
| 13 | Anti-CD138 | SASQGINNYLN |
| | LCDR1 | |
| 14 | Anti-CD138 | YTSTLQS |
| | LCDR2 | |
| 15 | Anti-CD138 | QQYSKLPRT |
| | LCDR3 | |
| 16 | Anti-CD16 | TSGMGVG |
| | HCDR1 | |
| 17 | Anti-CD16 | HIWWDDDKRYNPALKS |
| | HCDR2 | |
| 18 | Anti-CD16 | INPAWFAY |
| | HCDR3 | |
| 19 | Anti-CD16 | KASQSVDFDGDSFMN |
| | LCDR1 | |
| 20 | Anti-CD16 | TTSNLES |
| | LCDR2 | |
| 21 | Anti-CD16 | QQSNEDPYT |
| | LCDR3 | |
| 34 | Anti-CD3 | RYTMH |
| | HCDR1 | |
| 35 | Anti-CD3 | YINPSRGYTNYNQKFKD |
| | HCDR2 | |
| 36 | Anti-CD3 | YYDDHYCLDY |
| | HCDR3 | |
| 37 | Anti-CD3 | SASSSVSYMN |
| | LCDR1 | |
| 38 | Anti-CD3 | DTSKLAS |
| | LCDR2 | |
| 39 | Anti-CD3 | QQWSSNPFT |
| | LCDR3 | |
| 46 | Anti-CD80 | GYYYWT |
| | HCDR1-A | |
| 47 | Anti-CD80 | HIYGNGATTNYNPSLKS |
| | HCDR2-A | |
| 48 | Anti-CD80 | GPRPDCTTICYGGWVDVWGPGDLVTVSS |
| | HCDR3-A | |
| 49 | Anti-CD80 | GGDNSRNEYVH |
| | LCDR1-A | |
| 50 | Anti-CD80 | DDSDRPS |
| | LCDR2-A | |
| 51 | Anti-CD80 | QVWDRASDHPV |
| | LCDR3-A | |
| 52 | Anti-CD80 | DHYMY |
| | HCDR1-B | |
| 53 | Anti-CD80 | FIRNKPNGGTTEYAASVKD |
| | HCDR2-B | |
| 54 | Anti-CD80 | SYISHCRGGVCYGGYFEF |
| | HCDR3-B | |
| 55 | Anti-CD80 | RSSQSLKHSNGDTFLS |
| | LCDR1-B | |
| 56 | Anti-CD80 | KVSNRDS |
| | LCDR2-B | |
| 57 | Anti-CD80 | GQGTRTPPT |
| | LCDR3-B | |
| 58 | Anti-CD80 | GGYGWG |
| | HCDR1-C | |
| 59 | Anti-CD80 | SFYSSSGNTYYNPSLKS |
| | HCDR2-C | |
| 60 | Anti-CD80 | DRLFSVVGMVYNNWFDV |
| | HCDR3-C | |
| 61 | Anti-CD80 | TGSTSNIGGYDLH |
| | LCDR1-C | |
| 62 | Anti-CD80 | DINKRPS |
| | LCDR2-C | |
| 63 | Anti-CD80 | QSYDSSLNAQV |
| | LCDR3-C | |

A sequence of the antibody or antigen binding fragment thereof, including a heavy chain variable domain sequence, light chain variable domain sequence, or CDR sequence, can have at least 70% homology, at least 71% homology, at least 72% homology, at least 73% homology, at least 74% homology, at least 75% homology, at least 76% homology, at least 77% homology, at least 78% homology, at least 79% homology, at least 80% homology, at least 81% homology, at least 82% homology, at least 83% homology, at least 84% homology, at least 85% homology, at least 86% homology, at least 87% homology, at least 88% homology, at least 89% homology, at least 90% homology, at least 91% homology, at least 92% homology, at least 93% homology, at least 94% homology, at least 95% homology, at least 96% homology, at least 97% homology, at least 98% homology, at least 99% homology, at least 99.1% homology, at least 99.2% homology, at least 99.3% homology, at least 99.4% homology, at least 99.5% homology, at least 99.6% homology, at least 99.7% homology, at least 99.8% homology, at least 99.9% homology, at least 99.91% homology, at least 99.92% homology, at least 99.93% homology, at least 99.94% homology, at least 99.95% homology, at least 99.96% homology, at least 99.97% homology, at least 99.98% homology, or at least 99.99% homology to an amino acid or nucleic acid sequence disclosed herein (e.g., in Table 1).

Bi-specific Natural Killer and Neutrophil engager (BiKE, e.g., CD138-CD16 BiKE) is an example of an immunomodulatory transgene that can be introduced into the MYXV genome. BiKE (CD138-CD16) can direct Natural Killer (NK) cells and neutrophils to attack tumor targets, for example, by binding CD16 on the surface of NK cells and neutrophils, and binding CD138 on the surface of multiple myeloma (MM) cells. This can lead to NK/neutrophil activation, induction of target cancer cell apoptosis, and production of cytokines and chemokines in response to malignant targets (Gleason M K, Verneris M R, Todhunter D A, Zhang B, McCullar V, Zhou S X, et al. Bispecific and trispecific killer cell engagers directly activate human NK cells through CD16 signaling and induce cytotoxicity and cytokine production. Mol Cancer Ther 2012; 11(12):2674-84).

Bi-specific T cell engager (BiTE, e.g., CD138-CD3 BiTE) is an example of an immunomodulatory transgene that can be introduced into the MYXV genome. BiTE (CD138-CD3) can direct T cells to attack tumor targets, for example, by binding CD3 on the surface of T cells, and binding CD138 on the surface of MM cells. This can lead to T cell activation, induction of target cancer cell apoptosis or lysis, and production of cytokines and chemokines in response to malignant targets.

Membrane-integrated T cell engager (MiTE, e.g., anti-CD3 MiTE) is an example of an immunomodulatory transgene that can be introduced into the MYXV genome. MiTE can direct T cells to attack tumor targets, for example, by selective expression of MiTE on cancer cells susceptible to a MYXV of the disclosure, and binding CD3 on the surface of T cells. This can lead to T cell activation, induction of target cancer cell apoptosis or lysis, and production of cytokines and chemokines in response to malignant targets. A MiTE can comprise a transmembrane sequence. A MiTE can comprise a transmembrane sequence that was known at the time of the disclosure. Examples of transmembrane sequences include, but are not limited to, those provided in Table 2.

TABLE 2
examples of transmembrane sequences

| SEQ | | |
|---|---|---|
| ID NO: | Description | Sequence |
| 64 | CD8 hinge and | TTTPAPRPPTPAPTIASQPLSLR |
| | transmembrane | PEACRPAAGGAVHTRGLDFACDI |
| | domain | YIWAPLAGTCGVLLLSLVITLYC |
| 65 | 4-1BB hinge and | PSPADLSPGASSVTPPAPAREPG |
| | transmembrane | HSPQIISFFLALTSTALLFLLFF |
| | domain | LTLRFSVV |
| 66 | 4-1BB hinge and | PSPADLSPGASSVTPPAPAREPG |
| | transmembrane | HSPQIISFFLALTSTALLFLLFF |
| | domain | LTLRFSVV |

Disclosed herein, in some embodiments, are recombinant MYXV constructs that are armed with one or more of multi-specific immune cell engagers to target blood cancers, including MM. In this disclosure, MYXV expressing the transgenes BiKE, BiTE, or MiTE selectively infect and kill cancer cells, including for example cancer cells from patients with refractory disease that are resistant to standard therapies. In addition, it is demonstrated that these virus constructs can compromise MM cell viability by promoting killing of cancer cells by immune cells. Notably, two kinds of MM cell killing can be observed: direct cytotoxic killing of virus-infected MM cells, plus “off-target” killing of uninfected MM cells. Without wishing to be bound by theory, killing of uninfected MM cells may be mediated by MYXV-activated immune cells resident in the patient samples, and/or by directing immune cells (e.g., T cells for BiTE and MiTE, neutrophils and natural killer cells for BiKE) to attack cancer cells.

A sequence of the disclosure can have at least 70% homology, at least 71% homology, at least 72% homology, at least 73% homology, at least 74% homology, at least 75% homology, at least 76% homology, at least 77% homology, at least 78% homology, at least 79% homology, at least 80% homology, at least 81% homology, at least 82% homology, at least 83% homology, at least 84% homology, at least 85% homology, at least 86% homology, at least 87% homology, at least 88% homology, at least 89% homology, at least 90% homology, at least 91% homology, at least 92% homology, at least 93% homology, at least 94% homology, at least 95% homology, at least 96% homology, at least 97% homology, at least 98% homology, at least 99% homology, at least 99.1% homology, at least 99.2% homology, at least 99.3% homology, at least 99.4% homology, at least 99.5% homology, at least 99.6% homology, at least 99.7% homology, at least 99.8% homology, at least 99.9% homology, at least 99.91% homology, at least 99.92% homology, at least 99.93% homology, at least 99.94% homology, at least 99.95% homology, at least 99.96% homology, at least 99.97% homology, at least 99.98% homology, or at least 99.99% homology to an amino acid or nucleic acid sequence disclosed herein.

A transgene (e.g., a BiKE, BiTE, or MiTE transgene) of the disclosure can encode an antigen-binding protein, for example, one or more variable regions or complementarity determining regions (CDRs) from an antibody. In some embodiments, a transgene (e.g., a BiKE, BiTE, or MiTE) of the disclosure comprises one or more single chain variable fragments (scFvs) derived from one or more antibodies. A scFv (single-chain variable fragment) is a fusion protein that can comprise the VH and VL domains of an antibody connected by a peptide linker. For example, a BiKE, BiTE, or MiTE transgene can comprise two scFvs to allow binding of two targets. In some embodiments, a BiKE, BiTE, or MiTE comprises three, four, five, or more scFvs. In some embodiments, a BiKE, BiTE, or MiTE comprises one scFv.

In some embodiments, the BiKE, BiTE or MiTE comprises one copy of an antigen-binding protein. In some embodiments, the BiKE, BiTE or MiTE comprises two, three, four, five, or more copies of an antigen-binding protein.

Antigen binding proteins can be engineered based on antibody variable regions or CDRs. The variable (V) regions of an antibody mediate antigen binding and define the specificity of a particular antibody for an antigen. The variable region comprises relatively invariant sequences called framework regions, and hypervariable regions, which differ considerably in sequence among antibodies of different binding specificities. Within hypervariable regions are amino acid residues that primarily determine the binding specificity of the antibody. Sequences comprising these residues are known as complementarity determining regions (CDRs). One antigen binding site of an antibody comprises six CDRs, three in the hypervariable regions of the light chain, and three in the hypervariable regions of the heavy chain. The CDRs in the light chain are designated L1, L2, and L3, while the CDRs in the heavy chain are designated H1, H2, and H3. CDRs can also be designated LCDR1, LCDR2, LCDR3, HCDR1, HCDR2, and HCDR3, respectively. The contribution of each CDR to antigen binding varies among antibodies. CDRs can vary in length. For example, CDRs are often 5 to 14 residues in length, but CDRs as short as 0 residues or as long as 25 residues or longer exist. Several methods can be used to predict or designate CDR sequences, for example, the Kabat, Chothia, IMGT, paratome, Martin, and AHo methods. These CDR prediction methods can use different numbering systems, for example, because sequence insertions and deletions are numbered differently.

An antigen-binding protein can comprise a portion of an antibody or an antigen-binding fragment thereof, for example, the antigen-binding or variable region of the intact antibody. Non-limiting examples of antibody fragments include Fab, Fab′, F(ab′)2, dimers and trimers of Fab conjugates, Fv, scFv, minibodies, dia-, tria-, and tetrabodies, and linear antibodies. Fab and Fab′ are antigen-binding fragments that can comprise the VH and CH domains of the heavy chain linked to the VL and CL domains of the light chain via a disulfide bond. A F(ab′)2 can comprise two Fab or Fab′ that are joined by disulfide bonds. A Fv can comprise the VH and VL domains held together by non-covalent interactions. A scFv (single-chain variable fragment) is a fusion protein that can comprise the VH and VL domains connected by a peptide linker. Manipulation of the orientation of the VH and VL domains and the linker length can be used to create different forms of molecules that can be monomeric, dimeric (diabody), trimeric (triabody), or tetrameric (tetrabody). An antigen-binding protein can comprise a non-antibody-based protein, or an antigen-binding fragment thereof, for example, a DARPin.

In some embodiments, a transgene of the disclosure can encode a linker sequence (e.g., a linker sequence between different domains of a protein encoded by the transgene). In some embodiments, a linker is used to join antibody variable regions to form an scFv. In some embodiments, a linker is used to join two scFvs to form a BiKE, BiTE, or MiTE. A linker sequence can be, for example, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acid residues in length. In some embodiments, a linker is at least 1, at least 3, at least 5, at least 7, at least 9, at least 11, or at least 15 amino acids in length. In some embodiments, a linker is at most 5, at most 7, at most 9, at most 11, at most 15, at most 20, at most 25, or at most 50 amino acids in length.

A flexible linker can have a sequence containing stretches of glycine and serine residues. The small size of the glycine and serine residues provides flexibility, and allows for mobility of the connected functional domains. The incorporation of serine or threonine can maintain the stability of the linker in aqueous solutions by forming hydrogen bonds with the water molecules, thereby reducing unfavorable interactions between the linker and protein moieties. Flexible linkers can also contain additional amino acids such as threonine and alanine to maintain flexibility, as well as polar amino acids such as lysine and glutamine to improve solubility. A rigid linker can have, for example, an alpha helix-structure. An alpha-helical rigid linker can act as a spacer between protein domains. A linker can comprise any of the sequences in Table 3, or repeats thereof (e.g., 2, 3, 4, 5, 6, 7, 8, 9, or 10 repeats of any of SEQ ID NOs: 22-31). SEQ ID NOs: 22-27 provide flexible linkers. SEQ ID NOs: 28-31 provide rigid linkers.

TABLE 3

| SEQ ID NO: | Sequence |
|---|---|
| 22 | GGGGS |
| 23 | GGGS |
| 24 | GG |
| 25 | KESGSVSSEQLAQFRSLD |
| 26 | EGKSSGSGSESKST |
| 27 | GSAGSAAGSGEF |
| 28 | EAAAK |
| 29 | EAAAR |
| 30 | PAPAP |
| 31 | AEAAAKEAAAKA |

In some embodiments, a MYXV of the disclosure encodes one multi-specific immune cell engager. In some embodiments, a MYXV of the disclosure encodes two multi-specific immune cell engagers. In some embodiments, a MYXV of the disclosure encodes three multi-specific immune cell engagers. In some embodiments, a MYXV of the disclosure encodes four multi-specific immune cell engagers. In some embodiments, a MYXV of the disclosure encodes five multi-specific immune cell engagers.

In some embodiments, a MYXV of the disclosure can comprise one or more additional transgenes (e.g., one or more transgenes that are not multi-specific immune cell engagers).

In some embodiments, a MYXV of the disclosure can comprise one or more reporter transgenes (e.g., one or more reporter transgenes in addition to one or more of BiKE, BiTE, and BiTE). A reporter transgene (or reporter gene) can be used to monitor or quantify a MYXV in vitro, ex vivo, or in vivo. In some embodiments, a reporter transgene can be used to identify cells infected by an MYXV of the disclosure. For example, a MYXV of the disclosure can express a fluorescent transgene, and infected cells can be identified via fluorescence (e.g., fluorescence microscopy or flow cytometry). In some embodiments, a reporter transgene can be used to quantify cells infected by an MYXV of the disclosure. For example, a MYXV of the disclosure can express a fluorescent transgene, and infected cells can be quantified via fluorescence (e.g., quantification of the number or proportion of infected cells via fluorescence microscopy or flow cytometry). In some embodiments, a reporter transgene can be used to quantify viral replication or viral load in cells infected by an MYXV of the disclosure. For example, a MYXV of the disclosure can express a fluorescent transgene, and infected cells can be quantified via fluorescence (e.g., quantification of the average fluorescence intensity of cells via flow cytometry of fluorescence microscopy). In some embodiments, a MYXV of the disclosure can express a reporter gene that can be used for quantifying viral load or viral replication in vivo (e.g., imaging using an in vivo imaging system (IVIS)).

A reporter transgene of the disclosure can be expressed constitutively (e.g., under control of a constitutive promoter). A reporter transgene of the disclosure can be expressed conditionally (e.g., expressed under the control of a conditional promoter, e.g., a promoter that is only active or is more active in certain phases of a replication cycle).

Non-limiting examples of reporter transgenes include fluorescent proteins (e.g., green fluorescent protein (GFP), TdTomato, cyan fluorescent protein (CFP), yellow fluorescent protein (YFP), red fluorescent protein (RFP), Verde fluorescent protein (VFP), kindling fluorescent protein (KFP), mCherry, mTangerine, mRaspberry, mPlum, DsRed, etc.) and enzymes and substrates involved in luminescence (e.g., luciferin and/or luciferase).

In some embodiments, a MYXV of the disclosure does not comprise or encode a reporter transgene (e.g., does not encode any fluorescent or luminescent proteins).

In addition to expression of one or multi-specific immune cell engagers, a MYXV of the disclosure can be modified to carry one or more other genes that can enhance the anticancer effect of the MYXV treatment. Such a gene may be a gene that is involved in triggering apoptosis, or is involved in targeting the infected cell for immune destruction, such as a gene that restores responsiveness of the cell to interferon, or that results in the expression of a cell surface marker that stimulates an antibody response, such as a bacterial cell surface antigen. A MYXV of the disclosure can be modified to express one or more genes involved in shutting off the neoplastic or cancer cell's proliferation and growth, thereby preventing or reducing division of the cancer cell. In some embodiments, a MYXV of the disclosure can be modified to include therapeutic genes, such as genes involved in the synthesis of chemotherapeutic agents. In some embodiments, a MYXV of the disclosure can comprise a transgene that increases viral replication in cells of a particular species (for example, increased replication in human cancer cells for increased killing and inhibition of human cancer cells).

### Methods of Treatment

Provided herein, in some embodiments, are methods of treating a hematological cancer in a subject utilizing a myxoma virus (MYXV) of the disclosure. The hematological cancer can be a hematological cancer that comprises minimal residual disease (MRD) and/or drug-resistant MRD.

As disclosed in the Examples below, in vitro studies have demonstrated the ability of MYXV constructs of the disclosure to significantly eliminate refractory primary human multiple myeloma (MM) cells from patients who have failed standard therapies. Studies performed with MYXV have shown it can be a highly specific anti-cancer agent with a tropism for a number of human and murine cancer types.

Treatments of the disclosure (e.g., treatments utilizing MYXV-BiKE, MYXV-BiTE, or MYXV-MiTE) can comprise a number of novel and advantageous aspects. For example, these virus constructs selectively target and directly eliminate drug-resistant primary human MM cells that have been directly infected by each virus (e.g., CD138+ cells that express a viral reporter gene, such as GFP+ or TdTomato+). In some embodiments, MYXV of the disclosure comprising transgenes can not only eliminate contaminating hematologic cancer cells by direct killing of virus-infected cells, but also can eliminate disease by enhanced “off-target” killing of uninfected cancer cells (e.g., via immune cells directed to engage the cancer cells via BiTE, MiTE or BiKE). In some embodiments, MYXV of the disclosure comprising transgenes can elicit increased killing of uninfected cancer cells compared to other viruses (e.g., unarmed viruses or viruses lacking the multi-specific immune cell engager transgenes). In some embodiments, MYXV of the disclosure can exhibit enhanced “off-target” killing of uninfected MM cells (e.g., CD138+ cells that are negative for a viral reporter gene, such as GFP- or TdTomato-). Without wishing to be bound by any specific theory, virus-enhanced killing of uninfected cells may be mediated by immune cells directed to engage the cancer cells by the multi-specific immune cell engager.

In some embodiments a MYXV of the disclosure that expresses a multi-specific immune cell engager of the disclosure (e.g., a BiKE, MiTE, or BiTE) can increase killing of infected cancer cells (e.g., “on-target” killing). Killing of infected cancer cells can be increased, for example, by an engineered MYXV that expresses a multi-specific immune cell engager relative to a MYXV that does not express the multi-specific immune cell engager, or relative to uninfected cancer cells. A MYXV of the disclosure can increase killing of infected cancer cells, for example, by at least 5%, at least 10%, at least 20%, at least 30%, at least 40%, at least 50%, at least 75%, at least 2-fold, at least three-fold, at least five-fold, or at least ten-fold, e.g., as determined by a live/dead staining assay. The MYXV can preferentially infect and preferentially kill cancer cells over non-cancer cells.

In some embodiments a MYXV of the disclosure that expresses a multi-specific immune cell engager of the disclosure (e.g., a BiKE, MiTE, or BiTE) can increase killing of uninfected cancer cells (e.g., “off-target” killing). Killing of uninfected cancer cells can be increased, for example, by a MYXV that expresses a multi-specific immune cell engager relative to a MYXV that does not express the multi-specific immune cell engager, or relative to uninfected cancer cells. A MYXV of the disclosure can increase killing of uninfected cancer cells, for example, by at least 5%, at least 10%, at least 20%, at least 30%, at least 40%, at least 50%, at least 75%, at least 2-fold, at least three-fold, at least five-fold, or at least ten-fold, e.g., as determined by a live/dead staining assay. The increased killing of uninfected cancer cells can be mediated, for example, by immune cells that are directed to the cancer cells (e.g., T cells, NK cells, neutrophils, or other immune cells disclosed herein).

The use of MYXV expressing a multi-specific immune cell engager (e.g., BiKE, BiTE, or MiTE) to treat hematologic malignancies (e.g., refractory and/or minimal residual disease (MRD) of hematologic malignancies) can comprise multiple advantages over current therapies including chemotherapy and stem cell transplantation, and over other candidate oncolytic viruses. MYXV comprises a limited tropism that can, for example, allow the virus to infect human cancer cells, but not allow the virus to infect non-cancerous human cells. Unlike most viruses adapted from human pathogens, MYXV does not cause disease in humans, making it safe even for those patients with compromised immune systems. The lack of pre-existing anti-MYXV adaptive immunity in the human population can be advantageous, for example, allowing the virus to infect and kill cancer cells without being cleared as rapidly as a virus adapted from a human pathogen.

In ex vivo treatment approaches disclosed herein, incubation of MYXV with cells (e.g., bone marrow (BM) cells and/or peripheral blood mononuclear cells (PBMCs)) can be fast, for example, requiring only 1 hour of virus incubation ex vivo before re-infusion of the cells back into the cancer patient.

Thus, aspects of the present disclosure provide a method for inhibiting and/or treating a hematological cancer in a subject in need thereof. In certain embodiments, the method includes administering to a subject, such as a human subject, a MYXV of the disclosure that expresses one or more multi-specific immune cell engagers, such as BiKE, BiTE, or MiTE, thereby treating and/or inhibiting the hematological cancer in the subject in need thereof. The subject can be a mammal. The subject can be a human.

In some embodiments, the MYXV comprises MYXV-BiTE. In some embodiments, the MYXV comprises MYXV-MiTE. In some embodiments, the MYXV comprises MYXV-BiKE.

In some embodiments, the MYXV comprises BiTE and MiTE. In some embodiments, the MYXV comprises BiTE and BiKE. In some embodiments, the MYXV comprises MiTE and BiKE. In some embodiments, the MYXV comprises MiTE, BiTE, and BiKE. The MiTE, BiTE, or BiKE can comprise sequences from a human, a mouse, a mammal, or a combination thereof, and can comprise any of the sequences disclosed herein.

In some embodiments, an MYXV of the disclosure comprises a reporter transgene (e.g., a fluorescent protein or a luminescent substrate or enzyme). In some embodiments, an MYXV of the disclosure comprises one or more of BiKE, BiTE, and MiTE, and further comprises a reporter transgene. In some embodiments, an MYXV of the disclosure comprises one or more of BiKE, BiTE, and MiTE, and does not comprise a reporter transgene.

In some embodiments, a MYXV of the disclosure comprises a modification, insertion, deletion, or disruption in one or more genes in the viral genome. For example, a MYXV of the disclosure can comprise a modification, insertion, deletion, or disruption in or adjacent to any one or more of the M001R, M002R, M003.1R, M003.2R, M004.1R, M004R, M005R, M006R, M007R, M008.1R, M008R, M009L, M013, M036L, M063L, M11L, M128L, M131R, M135R, M136R, M141R, M148R, M151R, M152R, M153R, M154L, M156R, M-T2, M-T4, M-T5, M-T7, and SOD genes. In some embodiments, a deletion or disruption of a viral gene in a MYXV of the disclosure can reduce the ability of the virus to cause disease in a host animal, modulate host cell tropism, reduce innate immune evasion in non-cancer cells, modulate immune signaling in infected cells, modulate a cell death pathway in infected cells, increase viral replication in a cancer cells, or a combination thereof. In some embodiments, a MYXV of the disclosure comprises a modification, insertion, deletion, or disruption in the M153 gene.

In some embodiments, a MYXV of the disclosure comprises a modification, insertion, deletion, or disruption in the SOD gene. In some embodiments, a MYXV of the disclosure comprises a deletion or disruption in the SOD gene.

In some embodiments, a MYXV of the disclosure comprises one or more insertions, deletions, or substitutions within or adjacent to one or more genes associated with host cell tropism (for example, rabbit cell tropism). In some embodiments, one or more genes associated with rabbit cell tropism comprises M011L, M063, M135R, M136R, M-T2, M-T4, M-T5, M-T7, or a combination thereof. In some instances, the one or more genes associated with rabbit cell tropism comprise M135R, M136R, or a combination thereof.

In some embodiments, a MYXV of the disclosure comprises a modification, insertion, deletion, or disruption in the M135R gene. In some embodiments, a MYXV of the disclosure comprises a deletion or disruption in the M135R gene. A deletion or disruption of the M135R gene can, for example, attenuate the ability of a MYXV of the disclosure to cause disease in a host animal, without impairing the ability of the MYXV to exhibit an anti-cancer effect (e.g., infect and kill cancer cells, elicit an anti-tumor immune response, or a combination thereof).

MYXV can infect cells (e.g., human cells) that have a deficient innate anti-viral response. Having “a deficient innate anti-viral response” as used herein can refer to a cell that, when exposed to a virus or when invaded by a virus, fails to induce one or more anti-viral defense mechanisms. For example, a deficient innate anti-viral response can comprise failure to inhibit viral replication, failure to produce an anti-viral cytokine (e.g., an interferon), failure to respond to an anti-viral cytokine (e.g., induce an interferon response pathway), failure to induce apoptosis, failure to trigger recognition via an innate immune receptor (e.g., pattern recognition receptor), or a combination thereof.

A deficient innate anti-viral response may be caused by various factors, for example, malignant transformation, mutation, infection, genetic defect, or environmental stress.

In some embodiments, a MYXV of the disclosure is not administered to a subject comprising a deficient innate anti-viral response caused by a genetic defect, environmental stress, or an infection (e.g., a pre-existing infection with a different pathogen).

In some embodiments, a MYXV of the disclosure is administered to a subject comprising a deficient innate anti-viral response caused by malignant transformation (e.g., a cancer). A cell comprising a deficient innate anti-viral response can be a cancer cell, e.g., a cancer cell that has a reduced or defective innate anti-viral response upon exposure to or infection by a virus as compared to a normal cell, for example, a non-cancer cell. This can include, for example, a cancer cell that is non-responsive to interferon (e.g., type I interferon), and/or a cancer cell that has a reduced or defective apoptotic response or induction of the apoptotic pathway. In some embodiments of the method, an MYXV of the disclosure is capable of infecting a cell that has a deficient innate anti-viral response. In some embodiments, the cell is a mammalian cancer cell. In some embodiments, the cell is a human cancer cell, e.g., a human hematological cancer cell.

In some embodiments, a MYXV of the disclosure is used to treat a cancer. The examples provided herein for multiple myeloma are, by extension, applicable to other hematological cancers. Types of cancer that may be treated according to the disclosed method include, but are not limited to, hematological cancers such as leukemia, lymphoma, and myeloma, for example: multiple myeloma (MM); active multiple myeloma; smoldering multiple myeloma; plasmacytoma; solitary plasmacytoma of the bone; extramedullary plasmacytoma; light chain myeloma; non-secretory myeloma; immunoglobulin G (IgG) myeloma; immunoglobulin A (IgA) myeloma; immunoglobulin M (IgM) myeloma; immunoglobulin D (IgD) myeloma; immunoglobulin E (IgE) myeloma; hyperdiploid multiple myeloma; non-hyperdiploid multiple myeloma; Hodgkin lymphoma; non-Hodgkin lymphoma; acute lymphoblastic leukemia; acute myeloid leukemia; essential thrombocythemia; polycythemia vera; primary myelofibrosis; systemic mastocytosis; chronic myeloid leukemia; chronic neutrophilic leukemia; chronic eosinophilic leukemia; refractory anemia with ringed sideroblasts; refractory cytopenia with multilineage dysplasia; refractory anemia with excess blasts; type 1; refractory anemia with excess blasts; type 2; myelodysplastic syndrome (MDS) with isolated del (5q); MDS unclassifiable; chronic myelomonocytic leukemia (CML); atypical chronic myeloid leukemia; juvenile myelomonocytic leukemia; myeloproliferative/myelodysplastic syndromes-unclassifiable; B lymphoblastic leukemia/lymphoma; T lymphoblastic leukemia/lymphoma; diffuse large B-cell lymphoma; primary central nervous system lymphoma; primary mediastinal B-cell lymphoma; Burkitt lymphoma/leukemia; follicular lymphoma; chronic lymphocytic leukemia (CLL)/small lymphocytic lymphoma; B-cell prolymphocytic leukemia; lymphoplasmacytic lymphoma/Waldenstrom macroglobulinemia; Mantle cell lymphoma; marginal zone lymphomas; post-transplant lymphoproliferative disorders; HIV-associated lymphomas; primary effusion lymphoma; intravascular large B-cell lymphoma; primary cutaneous B-cell lymphoma; hairy cell leukemia; and monoclonal gammopathy of unknown significance; Anaplastic large cell lymphoma, Angioimmunoblastic T-cell lymphoma, Hepatosplenic T-cell lymphoma, B-cell lymphoma, reticuloendotheliosis, reticulosis, Mucosa-associated lymphatic tissue lymphoma, B-cell chronic lymphocytic leukemia, Waldenström's macroglobulinemia, Lymphomatoid granulomatosis, Nodular lymphocyte predominant Hodgkin's lymphoma, plasma cell leukemia, Acute erythraemia and erythroleukaemia, Acute erythremic myelosis, Acute erythroid leukemia, Heilmeyer-Schoner disease, Acute megakaryoblastic leukemia, Mast cell leukemia, Panmyelosis, Acute panmyelosis with myelofibrosis, Lymphosarcoma cell leukemia, Stem cell leukemia, Chronic leukaemia of unspecified cell type, Subacute leukaemia of unspecified cell type, Accelerated phase chronic myelogenous leukemia, Acute promyelocytic leukemia, Acute basophilic leukemia, Acute eosinophilic leukemia, Acute monocytic leukemia, Acute myeloblastic leukemia with maturation, Acute myeloid dendritic cell leukemia, Adult T-cell leukemia/lymphoma, Aggressive NK-cell leukemia, B-cell chronic lymphocytic leukemia, B-cell leukemia, Chronic myelogenous leukemia, Chronic idiopathic myelofibrosis, Kahler's disease, Myelomatosis, Solitary myeloma, Plasma cell leukemia, Angiocentric immunoproliferative lesion, Lymphoid granulomatosis, Angioimmunoblastic lymphadenopathy, T-gamma lymphoproliferative disease, Waldenström's macroglobulinaemia, Alpha heavy chain disease, Gamma heavy chain disease, and Franklin's disease. In some embodiments, the hematological cancer is multiple myeloma. In some embodiments, the cancer is a hematological cancer. In certain embodiments, the cancer comprises multiple myeloma.

Provided herein, in some embodiments, are methods of treating a hematological cancer (e.g., inhibiting, alleviating, stabilizing, reducing, or delaying progression of a hematological cancer). In some embodiments, the methods comprise administering a MYXV of the disclosure to a subject in need thereof to treat the hematological cancer. In some embodiments, the method further includes selecting a subject, such as a human subject, that has or is suspected of having a hematological cancer.

A MYXV of the disclosure can be administered in an amount effective to treat the hematological cancer. The amount may be sufficient to reduce the number of cancer cells in the subject (e.g., the concentration of the cancer cells in the subject's blood).

The effective amount to be administered to a subject can vary depending on many factors such as the pharmacodynamic properties of the MYXV, the modes of administration, the age, health and weight of the subject, the nature and extent of the disease state, the frequency of the treatment and the type of concurrent treatment, if any, and the virulence and titer of the virus.

The MYXV may be administered initially in a suitable amount that may be adjusted as required, depending on the clinical response of the subject. The effective amount of virus can be determined empirically and depends on the maximal amount of the MYXV that can be administered safely, and the minimal amount of the virus that produces the desired result.

To produce the same clinical effect when administering the virus systemically as that achieved through injection of the virus at the disease site, administration of significantly higher amounts of virus may be required. However, the appropriate dose level should be the minimum amount that would achieve the desired result.

The concentration of virus to be administered will vary depending on the virulence of the particular strain of MYXV that is to be administered and on the nature of the cells that are being targeted. In one embodiment, a dose of less than about 3×10\hat{ }10 focus forming units (“ffu”) or plaque forming units (“pfu”), also called “infectious units”, is administered to a human subject, in various embodiments, between about 10\hat{ }2 to about 10\hat{ }9 pfu, between about 10\hat{ }2 to about 10\hat{ }7 pfu, between about 10\hat{ }3 to about 10\hat{ }6 pfu, or between about 10\hat{ }4 to about 10\hat{ }5 pfu may be administered in a single dose.

In some embodiments, a subject is administered a certain dose of focus forming units (FFU) or plaque forming units (PFU) of a MYXV of the disclosure.

In some embodiments, the dose of MYXV administered to a subject is at least 1×10\hat{ }2, 2×10\hat{ }2, 3×10\hat{ }2, 4×10\hat{ }2, 5×10\hat{ }2, 6×10\hat{ }2, 7×10\hat{ }2, 8×10\hat{ }2, 9×10\hat{ }2, 1×10\hat{ }3, 2×10\hat{ }3, 3×10\hat{ }3, 4×10\hat{ }3, 5×10\hat{ }3, 6×10\hat{ }3, 7×10\hat{ }3, 8×10\hat{ }3, 9×10\hat{ }3, 1×10\hat{ }4, 2×10\hat{ }4, 3×10\hat{ }4, 4×10\hat{ }4, 5×10\hat{ }4, 6×10\hat{ }4, 7×10\hat{ }4, 8×10\hat{ }4, 9×10\hat{ }4, 1×10\hat{ }5, 2×10\hat{ }5, 3×10\hat{ }5, 4×10\hat{ }5, 5×10\hat{ }5, 6×10\hat{ }5, 7×10\hat{ }5, 8×10\hat{ }5, 9×10\hat{ }5, 1×10\hat{ }6, 2×10\hat{ }6, 3×10\hat{ }6, 4×10\hat{ }6, 5×10\hat{ }6, 6×10\hat{ }6, 7×10\hat{ }6, 8×10\hat{ }6, 9×10\hat{ }6, 1×10\hat{ }7, 2×10\hat{ }7, 3×10\hat{ }7, 4×10\hat{ }7, 5×10\hat{ }7, 6×10\hat{ }7, 7×10\hat{ }7, 8×10\hat{ }7, 9×10\hat{ }7, 1×10\hat{ }8, 2×10\hat{ }8, 3×10\hat{ }8, 4×10\hat{ }8, 5×10\hat{ }8, 6×10\hat{ }8, 7×10\hat{ }8, 8×10\hat{ }8, 9×10\hat{ }8, 1×10\hat{ }9, 2×10\hat{ }9, 3×10\hat{ }9, 4×10\hat{ }9, 5×10\hat{ }9, 6×10\hat{ }9, 7×10\hat{ }9, 8×10\hat{ }9, 9×10\hat{ }9, 1×10\hat{ }10, 2×10\hat{ }10, 3×10\hat{ }10, 4×10\hat{ }10, 5×10\hat{ }10, 6×10\hat{ }10, 7×10\hat{ }10, 8×10\hat{ }10, 9×10\hat{ }10, 1×10\hat{ }11, 2×10\hat{ }11, 3×10\hat{ }11, 4×10\hat{ }11, 5×10\hat{ }11, 6×10\hat{ }11, 7×10\hat{ }11, 8×10\hat{ }11, 9×10\hat{ }11, 1×10\hat{ }12, 2×10\hat{ }12, 3×10\hat{ }12, 4×10\hat{ }12, 5×10\hat{ }12, 6×10\hat{ }12, 7×10\hat{ }12, 8×10\hat{ }12, 9×10\hat{ }12, 1×10\hat{ }13, 2×10\hat{ }13, 3×10\hat{ }13, 4×10\hat{ }13, 5×10\hat{ }13, 6×10\hat{ }13, 7×10\hat{ }13, 8×10\hat{ }13, 9×10\hat{ }13, 1×10\hat{ }14, 2×10\hat{ }14, 3×10\hat{ }14, 4×10\hat{ }14, 5×10\hat{ }14, 6×10\hat{ }14, 7×10\hat{ }14, 8×10\hat{ }14, 9×10\hat{ }14, 1×10\hat{ }15, 2×10\hat{ }15, 3×10\hat{ }15, 4×10\hat{ }15, 5×10\hat{ }15, 6×10\hat{ }15, 7×10\hat{ }15, 8×10\hat{ }15, 9×10\hat{ }15, 1×10\hat{ }16, 2×10\hat{ }16, 3×10\hat{ }16, 4×10\hat{ }16, 5×10\hat{ }16, 6×10\hat{ }16, 7×10\hat{ }16, 8×10\hat{ }16, 9×10\hat{ }16, 1×10\hat{ }17, 2×10\hat{ }17, 3×10\hat{ }17, 4×10\hat{ }17, 5×10\hat{ }17, 6×10\hat{ }17, 7×10\hat{ }17, 8×10\hat{ }17, 9×10\hat{ }17, 1×10\hat{ }18, 2×10\hat{ }18, 3×10\hat{ }18, 4×10\hat{ }18, 5×10\hat{ }18, 6×10\hat{ }18, 7×10\hat{ }18, 8×10\hat{ }18, 9×10\hat{ }18, 1×10\hat{ }19, 2×10\hat{ }19, 3×10\hat{ }19, 4×10\hat{ }19, 5×10\hat{ }19, 6×10\hat{ }19, 7×10\hat{ }19, 8×10\hat{ }19, 9×10\hat{ }19, 1×10\hat{ }20, 2×10\hat{ }20, 3×10\hat{ }20, 4×10\hat{ }20, 5×10\hat{ }20, 6×10\hat{ }20, 7×10\hat{ }20, 8×10\hat{ }20, or 9×10\hat{ }20 FFU or PFU of a MYXV of the disclosure.

In some embodiments, the dose of MYXV administered to a subject is at most 1×10\hat{ }2, 2×10\hat{ }2, 3×10\hat{ }2, 4×10\hat{ }2, 5×10\hat{ }2, 6×10\hat{ }2, 7×10\hat{ }2, 8×10\hat{ }2, 9×10\hat{ }2, 1×10\hat{ }3, 2×10\hat{ }3, 3×10\hat{ }3, 4×10\hat{ }3, 5×10\hat{ }3, 6×10\hat{ }3, 7×10\hat{ }3, 8×10\hat{ }3, 9×10\hat{ }3, 1×10\hat{ }4, 2×10\hat{ }4, 3×10\hat{ }4, 4×10\hat{ }4, 5×10\hat{ }4, 6×10\hat{ }4, 7×10\hat{ }4, 8×10\hat{ }4, 9×10\hat{ }4, 1×10\hat{ }5, 2×10\hat{ }5, 3×10\hat{ }5, 4×10\hat{ }5, 5×10\hat{ }5, 6×10\hat{ }5, 7×10\hat{ }5, 8×10\hat{ }5, 9×10\hat{ }5, 1×10\hat{ }6, 2×10\hat{ }6, 3×10\hat{ }6, 4×10\hat{ }6, 5×10\hat{ }6, 6×10\hat{ }6, 7×10\hat{ }6, 8×10\hat{ }6, 9×10\hat{ }6, 1×10\hat{ }7, 2×10\hat{ }7, 3×10\hat{ }7, 4×10\hat{ }7, 5×10\hat{ }7, 6×10\hat{ }7, 7×10\hat{ }7, 8×10\hat{ }7, 9×10\hat{ }7, 1×10\hat{ }8, 2×10\hat{ }8, 3×10\hat{ }8, 4×10\hat{ }8, 5×10\hat{ }8, 6×10\hat{ }8, 7×10\hat{ }8, 8×10\hat{ }8, 9×10\hat{ }8, 1×10\hat{ }9, 2×10\hat{ }9, 3×10\hat{ }9, 4×10\hat{ }9, 5×10\hat{ }9, 6×10\hat{ }9, 7×10\hat{ }9, 8×10\hat{ }9, 9×10\hat{ }9, 1×10\hat{ }10, 2×10\hat{ }10, 3×10\hat{ }10, 4×10\hat{ }10, 5×10\hat{ }10, 6×10\hat{ }10, 7×10\hat{ }10, 8×10\hat{ }10, 9×10\hat{ }10, 1×10\hat{ }11, 2×10\hat{ }11, 3×10\hat{ }11, 4×10\hat{ }11, 5×10\hat{ }11, 6×10\hat{ }11, 7×10\hat{ }11, 8×10\hat{ }11, 9×10\hat{ }111, 1×10\hat{ }12, 2×10\hat{ }12, 3×10\hat{ }12, 4×10\hat{ }12, 5×10\hat{ }12, 6×10\hat{ }12, 7×10\hat{ }12, 8×10\hat{ }12, 9×10\hat{ }12, 1×10\hat{ }13, 2×10\hat{ }13, 3×10\hat{ }13, 4×10\hat{ }13, 5×10\hat{ }13, 6×10\hat{ }13, 7×10\hat{ }13, 8×10\hat{ }13, 9×10\hat{ }13, 1×10\hat{ }14, 2×10\hat{ }14, 3×10\hat{ }14, 4×10\hat{ }14, 5×10\hat{ }14, 6×10\hat{ }14, 7×10\hat{ }14, 8×10\hat{ }14, 9×10\hat{ }14, 1×10\hat{ }15, 2×10\hat{ }15, 3×10\hat{ }15, 4×10\hat{ }15, 5×10\hat{ }15, 6×10\hat{ }15, 7×10\hat{ }15, 8×10\hat{ }15, 9×10\hat{ }15, 1×10\hat{ }16, 2×10\hat{ }16, 3×10\hat{ }16, 4×10\hat{ }16, 5×10\hat{ }16, 6×10\hat{ }16, 7×10\hat{ }16, 8×10\hat{ }16, 9×10\hat{ }16, 1×10\hat{ }17, 2×10\hat{ }17, 3×10\hat{ }17, 4×10\hat{ }17, 5×10\hat{ }17, 6×10\hat{ }17, 7×10\hat{ }17, 8×10\hat{ }17, 9×10\hat{ }17, 1×10\hat{ }18, 2×10\hat{ }18, 3×10\hat{ }18, 4×10\hat{ }18, 5×10\hat{ }18, 6×10\hat{ }18, 7×10\hat{ }18, 8×10\hat{ }18, 9×10\hat{ }18, 1×10\hat{ }19, 2×10\hat{ }19, 3×10\hat{ }19, 4×10\hat{ }19, 5×10\hat{ }19, 6×10\hat{ }19, 7×10\hat{ }19, 8×10\hat{ }19, 9×10\hat{ }19, 1×10\hat{ }20, 2×10\hat{ }20, 3×10\hat{ }20, 4×10\hat{ }20, 5×10\hat{ }20, 6×10\hat{ }20, 7×10\hat{ }20, 8×10\hat{ }20, or 9×10\hat{ }20 FFU or PFU of a MYXV of the disclosure.

In some embodiments, a subject is administered a certain dose of focus forming units (FFU) or plaque forming units (PFU) of a MYXV of the disclosure per kilogram of body weight.

In some embodiments, the dose of MYXV administered to a subject is at least 1×10\hat{ }2, 2×10\hat{ }2, 3×10\hat{ }2, 4×10\hat{ }2, 5×10\hat{ }2, 6×10\hat{ }2, 7×10\hat{ }2, 8×10\hat{ }2, 9×10\hat{ }2, 1×10\hat{ }3, 2×10\hat{ }3, 3×10\hat{ }3, 4×10\hat{ }3, 5×10\hat{ }3, 6×10\hat{ }3, 7×10\hat{ }3, 8×10\hat{ }3, 9×10\hat{ }3, 1×10\hat{ }4, 2×10\hat{ }4, 3×10\hat{ }4, 4×10\hat{ }4, 5×10\hat{ }4, 6×10\hat{ }4, 7×10\hat{ }4, 8×10\hat{ }4, 9×10\hat{ }4, 1×10\hat{ }5, 2×10\hat{ }5, 3×10\hat{ }5, 4×10\hat{ }5, 5×10\hat{ }5, 6×10\hat{ }5, 7×10\hat{ }5, 8×10\hat{ }5, 9×10\hat{ }5, 1×10\hat{ }6, 2×10\hat{ }6, 3×10\hat{ }6, 4×10\hat{ }6, 5×10\hat{ }6, 6×10\hat{ }6, 7×10\hat{ }6, 8×10\hat{ }6, 9×10\hat{ }6, 1×10\hat{ }7, 2×10\hat{ }7, 3×10\hat{ }7, 4×10\hat{ }7, 5×10\hat{ }7, 6×10\hat{ }7, 7×10\hat{ }7, 8×10\hat{ }7, 9×10\hat{ }7, 1×10\hat{ }8, 2×10\hat{ }8, 3×10\hat{ }8, 4×10\hat{ }8, 5×10\hat{ }8, 6×10\hat{ }8, 7×10\hat{ }8, 8×10\hat{ }8, 9×10\hat{ }8, 1×10\hat{ }9, 2×10\hat{ }9, 3×10\hat{ }9, 4×10\hat{ }9, 5×10\hat{ }9, 6×10\hat{ }9, 7×10\hat{ }9, 8×10\hat{ }9, 9×10\hat{ }9, 1×10\hat{ }10, 2×10\hat{ }10, 3×10\hat{ }10, 4×10\hat{ }10, 5×10\hat{ }10, 6×10\hat{ }10, 7×10\hat{ }10, 8×10\hat{ }10, 9×10\hat{ }10, 1×10\hat{ }11, 2×10\hat{ }11, 3×10\hat{ }11, 4×10\hat{ }11, 5×10\hat{ }11, 6×10\hat{ }11, 7×10\hat{ }11, 8×10\hat{ }11, 9×10\hat{ }11, 1×10\hat{ }12, 2×10\hat{ }12, 3×10\hat{ }12, 4×10\hat{ }12, 5×10\hat{ }12, 6×10\hat{ }12, 7×10\hat{ }12, 8×10\hat{ }12, 9×10\hat{ }12, 1×10\hat{ }13, 2×10\hat{ }13, 3×10\hat{ }13, 4×10\hat{ }13, 5×10\hat{ }13, 6×10\hat{ }13, 7×10\hat{ }13, 8×10\hat{ }13, 9×10\hat{ }13, 1×10\hat{ }14, 2×10\hat{ }14, 3×10\hat{ }14, 4×10\hat{ }14, 5×10\hat{ }14, 6×10\hat{ }14, 7×10\hat{ }14, 8×10\hat{ }14, 9×10\hat{ }14, 1×10\hat{ }15, 2×10\hat{ }15, 3×10\hat{ }15, 4×10\hat{ }15, 5×10\hat{ }15, 6×10\hat{ }15, 7×10\hat{ }15, 8×10\hat{ }15, 9×10\hat{ }15, 1×10\hat{ }16, 2×10\hat{ }16, 3×10\hat{ }16, 4×10\hat{ }16, 5×10\hat{ }16, 6×10\hat{ }16, 7×10\hat{ }16, 8×10\hat{ }16, 9×10\hat{ }16, 1×10\hat{ }17, 2×10\hat{ }17, 3×10\hat{ }17, 4×10\hat{ }17, 5×10\hat{ }17, 6×10\hat{ }17, 7×10\hat{ }17, 8×10\hat{ }17, 9×10\hat{ }17, 1×10\hat{ }18, 2×10\hat{ }18, 3×10\hat{ }18, 4×10\hat{ }18, 5×10\hat{ }18, 6×10\hat{ }18, 7×10\hat{ }18, 8×10\hat{ }18, 9×10\hat{ }18, 1×10\hat{ }19, 2×10\hat{ }19, 3×10\hat{ }19, 4×10\hat{ }19, 5×10\hat{ }19, 6×10\hat{ }19, 7×10\hat{ }19, 8×10\hat{ }19, 9×10\hat{ }19, 1×10\hat{ }20, 2×10\hat{ }20, 3×10\hat{ }20, 4×10\hat{ }20, 5×10\hat{ }20, 6×10\hat{ }20, 7×10\hat{ }20, 8×10\hat{ }20, or 9×10\hat{ }20 FFU or PFU of a MYXV of the disclosure per kilogram of body weight of the subject.

In some embodiments, the dose of MYXV administered to a subject is at most 1×10\hat{ }2, 2×10\hat{ }2, 3×10\hat{ }2, 4×10\hat{ }2, 5×10\hat{ }2, 6×10\hat{ }2, 7×10\hat{ }2, 8×10\hat{ }2, 9×10\hat{ }2, 1×10\hat{ }3, 2×10\hat{ }3, 3×10\hat{ }3, 4×10\hat{ }3, 5×10\hat{ }3, 6×10\hat{ }3, 7×10\hat{ }3, 8×10\hat{ }3, 9×10\hat{ }3, 1×10\hat{ }4, 2×10\hat{ }4, 3×10\hat{ }4, 4×10\hat{ }4, 5×10\hat{ }4, 6×10\hat{ }4, 7×10\hat{ }4, 8×10\hat{ }4, 9×10\hat{ }4, 1×10\hat{ }5, 2×10\hat{ }5, 3×10\hat{ }5, 4×10\hat{ }5, 5×10\hat{ }5, 6×10\hat{ }5, 7×10\hat{ }5, 8×10\hat{ }5, 9×10\hat{ }5, 1×10\hat{ }6, 2×10\hat{ }6, 3×10\hat{ }6, 4×10\hat{ }6, 5×10\hat{ }6, 6×10\hat{ }6, 7×10\hat{ }6, 8×10\hat{ }6, 9×10\hat{ }6, 1×10\hat{ }7, 2×10\hat{ }7, 3×10\hat{ }7, 4×10\hat{ }7, 5×10\hat{ }7, 6×10\hat{ }7, 7×10\hat{ }7, 8×10\hat{ }7, 9×10\hat{ }7, 1×10\hat{ }8, 2×10\hat{ }8, 3×10\hat{ }8, 4×10\hat{ }8, 5×10\hat{ }8, 6×10\hat{ }8, 7×10\hat{ }8, 8×10\hat{ }8, 9×10\hat{ }8, 1×10\hat{ }9, 2×10\hat{ }9, 3×10\hat{ }9, 4×10\hat{ }9, 5×10\hat{ }9, 6×10\hat{ }9, 7×10\hat{ }9, 8×10\hat{ }9, 9×10\hat{ }9, 1×10\hat{ }10, 2×10\hat{ }10, 3×10\hat{ }10, 4×10\hat{ }10, 5×10\hat{ }10, 6×10\hat{ }10, 7×10\hat{ }10, 8×10\hat{ }10, 9×10\hat{ }10, 1×10\hat{ }11, 2×10\hat{ }11, 3×10\hat{ }11, 4×10\hat{ }11, 5×10\hat{ }11, 6×10\hat{ }11, 7×10\hat{ }11, 8×10\hat{ }11, 9×10\hat{ }11, 1×10\hat{ }12, 2×10\hat{ }12, 3×10\hat{ }12, 4×10\hat{ }12, 5×10\hat{ }12, 6×10\hat{ }12, 7×10\hat{ }12, 8×10\hat{ }12, 9×10\hat{ }12, 1×10\hat{ }13, 2×10\hat{ }13, 3×10\hat{ }13, 4×10\hat{ }13, 5×10\hat{ }13, 6×10\hat{ }13, 7×10\hat{ }13, 8×10\hat{ }13, 9×10\hat{ }13, 1×10\hat{ }14, 2×10\hat{ }14, 3×10\hat{ }14, 4×10\hat{ }14, 5×10\hat{ }14, 6×10\hat{ }14, 7×10\hat{ }14, 8×10\hat{ }14, 9×10\hat{ }14, 1×10\hat{ }15, 2×10\hat{ }15, 3×10\hat{ }15, 4×10\hat{ }15, 5×10\hat{ }15, 6×10\hat{ }15, 7×10\hat{ }15, 8×10\hat{ }15, 9×10\hat{ }15, 1×10\hat{ }16, 2×10\hat{ }16, 3×10\hat{ }16, 4×10\hat{ }16, 5×10\hat{ }16, 6×10\hat{ }16, 7×10\hat{ }16, 8×10\hat{ }16, 9×10\hat{ }16, 1×10\hat{ }17, 2×10\hat{ }17, 3×10\hat{ }17, 4×10\hat{ }17, 5×10\hat{ }17, 6×10\hat{ }17, 7×10\hat{ }17, 8×10\hat{ }17, 9×10\hat{ }17, 1×10\hat{ }18, 2×10\hat{ }18, 3×10\hat{ }18, 4×10\hat{ }18, 5×10\hat{ }18, 6×10\hat{ }18, 7×10\hat{ }18, 8×10\hat{ }18, 9×10\hat{ }18, 1×10\hat{ }19, 2×10\hat{ }19, 3×10\hat{ }19, 4×10\hat{ }19, 5×10\hat{ }19, 6×10\hat{ }19, 7×10\hat{ }19, 8×10\hat{ }19, 9×10\hat{ }19, 1×10\hat{ }20, 2×10\hat{ }20, 3×10\hat{ }20, 4×10\hat{ }20, 5×10\hat{ }20, 6×10\hat{ }20, 7×10\hat{ }20, 8×10\hat{ }20, or 9×10\hat{ }20 FFU or PFU of a MYXV of the disclosure per kilogram of body weight of the subject.

A MYXV of the disclosure can be administered at any interval desired. In some embodiments, the MYXV can be administered hourly. In some embodiments, the MYXV can be administered about every 1, 2, 3, 4, 5, 6, 7, 8, 9, 10, 11, 12, 13, 14, 15, 16, 18, 20, 22, 24, 26, 28, 30, 32, 36, 40, 44, or 48 hours. In some embodiments, the MYXV can be administered twice a day, once a day, five times a week, four times a week, three times a week, two times a week, once a week, once every two weeks, once every three weeks, once every four weeks, once a month, once every five weeks, once every six weeks, once every eight weeks, once every two months, once every twelve weeks, once every three months, once every four months, once every six months, once a year, or less frequently.

A MYXV of the disclosure can be administered to a subject in a therapeutically-effective amount by various forms and routes including, for example, systemic, oral, topical, parenteral, intravenous injection, intravenous infusion, intratumoral injection, subcutaneous injection, intramuscular injection, intradermal injection, intraperitoneal injection, intracerebral injection, subarachnoid injection, intraspinal injection, intrasternal injection, intraarticular injection, endothelial administration, local administration, intranasal administration, intrapulmonary administration, intraarterial administration, intrathecal administration, inhalation, intralesional administration, intradermal administration, epidural administration, absorption through epithelial or mucocutaneous linings (e.g., oral mucosa, rectal and intestinal mucosa), intracapsular administration, subcapsular administration, intracardiac administration, transtracheal administration, subcuticular administration, subarachnoid administration, subcapsular administration, intraspinal administration, or intrasternal administration.

In some embodiments, the virus is administered systemically. In some embodiments, the virus is administered by injection at a disease site. In some embodiments, the virus is administered orally. In some embodiments, the virus is administered parenterally.

A MYXV of the disclosure (e.g., expressing one or more multi-specific immune cell engagers, such as BiKE, BiTE and/or MiTE), can be administered as a sole therapy or can be administered in combination with one or more other therapies. In some embodiments, a MYXV of the disclosure is administered in combination with a chemotherapy, an immunotherapy, a cell therapy, a radiation therapy, a stem cell transplant (such as an autologous stem cell transplant), or a combination thereof. For example, the MYXV expressing one or more multi-specific immune cell engagers, such as BiKE, BiTE and/or MiTE may be administered either prior to or following another treatment, such as administration of radiotherapy or conventional chemotherapeutic drugs and/or a stem cell transplant, such as an autologous stem cell transplant or an allogenic stem cell transplant (e.g., a HLA-matched, HLA-mismatched, or haploidentical transplant).

In some embodiments, a MYXV of the disclosure can be in combination with an immune checkpoint modulator. Examples of immune checkpoint modulators include, but are not limited to, PD-L1 inhibitors such as durvalumab (Imfinzi) from AstraZeneca, atezolizumab (MPDL3280A) from Genentech, avelumab from EMD Serono/Pfizer, CX-072 from CytomX Therapeutics, FAZ053 from Novartis Pharmaceuticals, KN035 from 3D Medicine/Alphamab, LY3300054 from Eli Lilly, or M7824 (anti-PD-L1/TGFbeta trap) from EMD Serono; PD-L2 inhibitors such as GlaxoSmithKline's AMP-224 (Amplimmune), and rHIgM12B7; PD-1 inhibitors such as nivolumab (Opdivo) from Bristol-Myers Squibb, pembrolizumab (Keytruda) from Merck, AGEN 2034 from Agenus, BGB-A317 from BeiGene, B1-754091 from Boehringer-Ingelheim Pharmaceuticals, CBT-501 (genolimzumab) from CBT Pharmaceuticals, INCSHR1210 from Incyte, JNJ-63723283 from Janssen Research & Development, MEDIO680 from MedImmune, MGA 012 from MacroGenics, PDR001 from Novartis Pharmaceuticals, PF-06801591 from Pfizer, REGN2810 (SAR439684) from Regeneron Pharmaceuticals/Sanofi, or TSR-042 from TESARO; CTLA-4 inhibitors such as ipilimumab (also known as Yervoy®, MDX-010, BMS-734016 and MDX-101) from Bristol Meyers Squibb, tremelimumab (CP-675,206, ticilimumab) from Pfizer, or AGEN 1884 from Agenus; LAG3 inhibitors such as BMS-986016 from Bristol-Myers Squibb, IMP701 from Novartis Pharmaceuticals, LAG525 from Novartis Pharmaceuticals, or REGN3767 from Regeneron Pharmaceuticals; B7-H3 inhibitors such as enoblituzumab (MGA271) from MacroGenics; KIR inhibitors such as Lirilumab (IPH2101; BMS-986015) from Innate Pharma; CD137 inhibitors such as urelumab (BMS-663513, Bristol-Myers Squibb), PF-05082566 (anti-4-1BB, PF-2566, Pfizer), or XmAb-5592 (Xencor); and PS inhibitors such as Bavituximab. In some embodiments, the MYXV is combined with an antibody or antigen-binding fragment thereof, an RNAi molecule, or a small molecule, that acts on or is specific for, for example, TIM3, CD52, CD30, CD20, CD33, CD27, OX40, GITR, ICOS, BTLA (CD272), CD160, 2B4, LAIR1, TIGIT, LIGHT, DR3, CD226, CD2, or SLAM.

An MYXV of the disclosure can be prepared using standard techniques. For example, the virus may be prepared by infecting cultured rabbit cells, or immortalized permissive human or primate cells, with the MYXV strain that is to be used, allowing the infection to progress such that the virus replicates in the cultured cells and can be released by standard methods for disrupting the cell surface and thereby releasing the virus particles for harvesting. Once harvested, the virus titer may be determined, for example, by infecting a confluent lawn of rabbit cells and performing a plaque assay (see Mossman et al. (1996) Virology 215:17-30 which is hereby incorporated by reference in its entirety).

### Cellular Delivery of MYXV

Further disclosed herein, in some embodiments, is a novel delivery strategy where a MYXV of the disclosure is first adsorbed to cells, and the cells are administered to a subject. This method can deliver a MYXV of the disclosure to sites of disease via virus-bearing “carrier” cells. In some embodiments, this cell-assisted delivery of virus has the ability to reduce or eliminate tumor burden and increase survival of the subject.

The delivery of MYXV via carrier cells represents a new potential therapeutic regimen for hematological cancers. In some embodiments, a MYXV of the disclosure is adsorbed to leukocytes (for example, leukocytes from bone marrow and/or peripheral blood), and the leukocytes are infused into a subject. Pre-loading leukocytes with MYXV ex vivo prior to leukocyte infusion into a cancer-bearing recipient can be exploited for multiple myeloma (MM) and for any other hematologic cancers disclosed herein. In some embodiments, pre-loading leukocytes with MYXV ex vivo prior to leukocyte infusion into a cancer-bearing recipient can be effective for treating any cancer amenable to the localization and infiltration of the leukocytes into distant tumor sites.

In some embodiments, the combined “leukocyte/MYXV” therapy causes increased cancer cell death in the tumor beds to enhance anti-tumor immunogenicity. For example, in some embodiments a MYXV of the disclosure (e.g., a MYXV expressing one or more multi-specific immune cell engagers, such as BiKE, BiTE and/or MiTE) is delivered to cancer sites such as the bone marrow beds that harbor minimal residual disease (MRD), via migration of leukocytes pre-adsorbed or pre-infected with virus ex vivo. This systemic delivery method is sometimes called “ex vivo virotherapy”, or EVV (e.g., EV2), because the virus is first delivered to leukocytes prior to infusion into the patient.

In some embodiments, the cell-mediated delivery of MYXV increases the level of direct killing of infected hematological cancer cells, and, while not being bound by theory, acts as an activator of the host immune system, which can lead to long term regression of cancer. This can provide a new method of treatment of hematological cancers in the bone and/or lymph nodes, which has proved to be difficult with current treatments.

Thus, in certain embodiments, methods of the disclosure comprise administering to a subject with cancer leukocytes that comprise an adsorbed MYXV of the disclosure (e.g., a MYXV expressing one or more multi-specific immune cell engagers, such as BiKE, BiTE and/or MiTE), thereby treating and/or inhibiting the cancer in the subject. A MYXV of the disclosure can be adsorbed by exposing leukocytes to the MYXV under conditions that permit binding of the MYXV to the surface of the leukocytes.

In some embodiments, a MYXV of the disclosure is adsorbed to leukocytes (for example, leukocytes from bone marrow and/or peripheral blood), and the leukocytes are infused into a subject. The leukocytes can be from bone marrow (for example, from bone marrow aspirate or bone marrow biopsy). The leukocytes can be from blood (e.g., peripheral blood mononuclear cells). In some embodiments, the leukocytes are obtained from a subject, for example a subject that has cancer, adsorbed with MYXV, and re-infused into the subject (e.g., as an autologous cell transplant). In some embodiments, the leukocytes are obtained from one or more allogenic donors (for example, HLA-matched, HLA-mismatched, or haploidentical donors). In some embodiments, the leukocytes are obtained from an HLA-matched sibling.

The leukocytes can be sorted or purified by, for example, red blood cell lysis, density gradient centrifugation (e.g., Ficoll-Paque), leukapheresis, techniques comprising antibodies or derivatives thereof (e.g., positive or negative selection via fluorescent activated cell sorting or magnetic activated cell sorting), or any combination thereof, before or after a MYXV of the disclosure is adsorbed. In some embodiments, the leukocytes are sorted or purified to enrich for cancer cells before or after a MYXV of the disclosure is adsorbed (e.g., cells expressing a marker associated with a cancer, e.g., CD138 for multiple myeloma cells). In some embodiments, the leukocytes are sorted or purified to enrich for non-cancer cells before or after a MYXV of the disclosure is adsorbed. In some embodiments, the cells are sorted or purified to enrich for one or more cell subsets cells before or after a MYXV of the disclosure is adsorbed (e.g., monocytes, lymphocytes, B cells, plasma cells, T cells, neutrophils, basophils, eosinophils, megakaryocytes, NK cells, NKT cells, mast cells, innate lymphoid cells, common myeloid precursors, common lymphoid precursors, myeloblasts, monoblasts, promonocytes, lymphoblasts, prolymphocytes, hemocytoblasts, megakaryoblasts, promegakaryocytes, stem cells, pro B cells, pre B cells, precursors thereof, or any combination thereof). In some embodiments, a MYXV of the disclosure is adsorbed to the leukocytes, and the leukocytes are enriched for cells comprising the MYXV (e.g., with MYXV bound and/or internalized).

The leukocytes can be stored (for example, cryopreserved) prior to or after adsorbing an MYXV of the disclosure. In some embodiments, the leukocytes can be cryopreserved, and later thawed prior to infusion into a subject.

In some embodiments, the method comprises adsorbing a MYXV of the disclosure onto the surface of leukocytes (e.g., peripheral blood mononuclear cells, bone marrow cells, or a purified/enriched subset thereof). In some embodiments, adsorbing the myxoma virus onto the surface of the leukocytes comprises exposing the leukocytes to the MYXV under conditions that permit binding of the MYXV to the surface of the mononuclear peripheral blood cells and/or bone marrow cells. In some embodiments, the method includes infecting the leukocytes with a MYXV of the disclosure. In some embodiments, infecting the leukocytes with a MYXV of the disclosure comprises exposing the leukocytes to the MYXV under conditions that permit internalization of the MYXV into at least a portion of the leukocytes. Exposing leukocytes to MYXV can comprise any suitable reagents or conditions (e.g., sterile cell culture media, media supplements, and appropriate incubation conditions to allow adsorption and/or infection of the leukocytes, and maintain viability of the leukocytes).

The MYXV and leukocytes can be exposed to each other at any ratio that permits the virus to adsorb to the leukocytes. In some embodiments, adsorbing the myxoma virus onto the surface of the leukocytes comprises exposing the leukocytes to the MYXV at a multiplicity of infection (MOI) of about 0.000001, 0.00001, 0.0001, 0.0001, 0.001, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 150, 200, 250, 300, 400, 500, 600, 700, 800, 900, 1000, 2000, 3000, 4000, 5000, 6000, 7000, 8000, 9000, 1×10\hat{ }4, 1×10\hat{ }5, 1×10\hat{ }6, 1×10\hat{ }9, 1×10\hat{ }10, 1×10\hat{ }11, 1×10\hat{ }12, 1×10\hat{ }13, 1×10\hat{ }14, or 1×10\hat{ }15 viruses per leukocyte.

In some embodiments, adsorbing the myxoma virus onto the surface of the leukocytes comprises exposing the leukocytes to the MYXV at a multiplicity of infection (MOI) of at least 0.000001, at least 0.00001, at least 0.0001, at least 0.0001, at least 0.001, at least 0.01, at least 0.02, at least 0.03, at least 0.04, at least 0.05, at least 0.06, at least 0.07, at least 0.08, at least 0.09, at least 0.1, at least 0.2, at least 0.3, at least 0.4, at least 0.5, at least 0.6, at least 0.7, at least 0.8, at least 0.9, at least 1, at least 1.1, at least 1.2, at least 1.3, at least 1.4, at least 1.5, at least 1.6, at least 1.7, at least 1.8, at least 1.9, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 25, at least 30, at least 35, at least 40, at least 45, at least 50, at least 60, at least 70, at least 80, at least 90, at least 100, at least 150, at least 200, at least 250, at least 300, at least 400, at least 500, at least 600, at least 700, at least 800, at least 900, at least 1000, at least 2000, at least 3000, at least 4000, at least 5000, at least 6000, at least 7000, at least 8000, at least 9000, at least 1×10\hat{ }4, at least 1×10\hat{ }5, at least 1×10\hat{ }6, at least 1×10\hat{ }9, at least 1×10\hat{ }10, at least 1×10\hat{ }11, at least 1×10\hat{ }12, at least 1×10\hat{ }13, at least 1×10\hat{ }14, or at least 1×10\hat{ }15 viruses per leukocyte.

In some embodiments, adsorbing the myxoma virus onto the surface of the leukocytes comprises exposing the leukocytes to the MYXV at a multiplicity of infection (MOI) of at most 0.000001, at most 0.00001, at most 0.0001, at most 0.0001, at most 0.001, at most 0.01, at most 0.02, at most 0.03, at most 0.04, at most 0.05, at most 0.06, at most 0.07, at most 0.08, at most 0.09, at most 0.1, at most 0.2, at most 0.3, at most 0.4, at most 0.5, at most 0.6, at most 0.7, at most 0.8, at most 0.9, at most 1, at most 1.1, at most 1.2, at most 1.3, at most 1.4, at most 1.5, at most 1.6, at most 1.7, at most 1.8, at most 1.9, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 25, at most 30, at most 35, at most 40, at most 45, at most 50, at most 60, at most 70, at most 80, at most 90, at most 100, at most 150, at most 200, at most 250, at most 300, at most 400, at most 500, at most 600, at most 700, at most 800, at most 900, at most 1000, at most 2000, at most 3000, at most 4000, at most 5000, at most 6000, at most 7000, at most 8000, at most 9000, at most 1×10\hat{ }4, at most 1×10\hat{ }5, at most 1×10\hat{ }6, at most 1×10\hat{ }9, at most 1×10\hat{ }10, at most 1×10\hat{ }11, at most 1×10\hat{ }12, at most 1×10\hat{ }13, at most 1×10\hat{ }14, or at most 1×10\hat{ }15 viruses per leukocyte.

In some embodiments, adsorbing the myxoma virus onto the surface of the leukocytes comprises exposing the leukocytes to the MYXV at a multiplicity of infection (MOI) of between about, for example, 0.000001 to 1×10\hat{ }15, 0.0001 to 1×10\hat{ }6, 0.001 to 1×10\hat{ }4, 0.001 to 1000, 0.001 to 100, 0.001 to 10, 0.001 to 1, 0.001 to 0.1, 0.001 to 0.01, 0.01 to 1×10\hat{ }4, 0.01 to 1000, 0.01 to 100, 0.01 to 10, 0.01 to 1, 0.01 to 0.1, 0.1 to 1×10\hat{ }4, 0.1 to 1000, 0.1 to 100, 0.1 to 10, 0.1 to 1, 1 to 1×10\hat{ }4, 1 to 1000, 1 to 100, or 1 to 10 viruses per leukocyte.

In some embodiments, adsorbing the myxoma virus onto the surface of the leukocytes comprises exposing the leukocytes to the MYXV at a multiplicity of infection (MOI) of between about 0.1 to 10. In some embodiments, adsorbing the myxoma virus onto the surface of the leukocytes comprises exposing the leukocytes to the MYXV at a multiplicity of infection (MOI) of between about 0.01 to 100. In some embodiments, adsorbing the myxoma virus onto the surface of the leukocytes comprises exposing the leukocytes to the MYXV at a multiplicity of infection (MOI) of between about 0.001 to 1000.

In some embodiments, the leukocytes are contacted or adsorbed with a MYXV of the disclosure for a period of about 5 minutes, 10 minutes, 15 minutes, 20 minutes, 25 minutes, 30 minutes, 35 minutes, 40 minutes, 45 minutes, 50 minutes, 55 minutes, 60 minutes, 65 minutes, 70 minutes, 75 minutes, 80 minutes, 85 minutes, 90 minutes, 95 minutes, 100 minutes, 105 minutes, 110 minutes, 115 minutes, 120 minutes, 2.5 hours, 3 hours, 3.5 hours, 4 hours, 4.5 hours, 5 hours, 5.5 hours, 6 hours, 7 hours, 8 hours, 9 hours, 10 hours, 11 hours, 12 hours, 13 hours, 14 hours, 15 hours, 16 hours, 18 hours, 20 hours, 22 hours, or 24 hours.

In some embodiments, the leukocytes are contacted or adsorbed with a MYXV of the disclosure for a period of at least 5 minutes, at least 10 minutes, at least at least 15 minutes, at least 20 minutes, at least 25 minutes, at least 30 minutes, at least 35 minutes, at least 40 minutes, at least 45 minutes, at least 50 minutes, at least 55 minutes, at least 60 minutes, at least 65 minutes, at least 70 minutes, at least 75 minutes, at least 80 minutes, at least 85 minutes, at least 90 minutes, at least 95 minutes, at least 100 minutes, at least 105 minutes, at least 110 minutes, at least 115 minutes, at least 120 minutes, at least 2.5 hours, at least 3 hours, at least 3.5 hours, at least 4 hours, at least 4.5 hours, at least 5 hours, at least 5.5 hours, at least 6 hours, at least 7 hours, at least 8 hours, at least 9 hours, at least 10 hours, at least 11 hours, at least 12 hours, at least 13 hours, at least 14 hours, at least 15 hours, at least 16 hours, at least 18 hours, at least 20 hours, at least 22 hours, at least 24 hours, or more.

In some embodiments, the leukocytes are contacted or adsorbed with a MYXV of the disclosure for a period of at most 5 minutes, at most 10 minutes, at most at most 15 minutes, at most 20 minutes, at most 25 minutes, at most 30 minutes, at most 35 minutes, at most 40 minutes, at most 45 minutes, at most 50 minutes, at most 55 minutes, at most 60 minutes, at most 65 minutes, at most 70 minutes, at most 75 minutes, at most 80 minutes, at most 85 minutes, at most 90 minutes, at most 95 minutes, at most 100 minutes, at most 105 minutes, at most 110 minutes, at most 115 minutes, at most 120 minutes, at most 2.5 hours, at most 3 hours, at most 3.5 hours, at most 4 hours, at most 4.5 hours, at most 5 hours, at most 5.5 hours, at most 6 hours, at most 7 hours, at most 8 hours, at most 9 hours, at most 10 hours, at most 11 hours, at most 12 hours, at most 13 hours, at most 14 hours, at most 15 hours, at most 16 hours, at most 18 hours, at most 20 hours, at most 22 hours, at most 24 hours, or less.

In some embodiments, the BM or PBMC cells are contacted or adsorbed with MYXV constructs ex vivo for about one hour.

### ADDITIONAL Ex Vivo Methods

As disclosed herein, MYXV is capable of selectively infecting cells that have a deficient innate anti-viral response, and can be used as an indicator of such a deficiency in cells. Thus, cells removed from a subject may be assayed for deficiency in an innate anti-viral response using the methods of the present disclosure. Such determination may indicate, when combined with other indicators, that the subject may be suffering from a particular disease state, for example, cancer. The cells may be removed from a subject, including a human subject, using known biopsy methods. The biopsy method will depend on the location and type of cell that is to be tested. Cells can be cultured and exposed to MYXV, for example by adding live MYXV to the culture medium. The multiplicity of infection (MOI), may be varied to determine an optimum MOI for a given cell type, density and culture technique, using a positive control cell culture that is known to be infected upon exposure to MYXV.

The amount of MYXV added to the cultured cells can vary depending on cell type, method of culturing and strain of virus.

Infectivity of the cultured cells by MYXV can be determined by various methods known to a skilled person, including the ability of the MYXV to cause cell death. It can also involve the addition of reagents to the cell culture to complete an enzymatic or chemical reaction with a viral expression product. The viral expression product can be expressed from a reporter gene that has been inserted into the MYXV genome.

In one embodiment, the MYXV can be modified to enhance the ease of detection of infection state. For example, the MYXV can be genetically modified to express a marker that can be readily detected by phase contrast microscopy, fluorescence microscopy, or by radioimaging. The marker can be an expressed fluorescent protein or an expressed enzyme that may be involved in a colorimetric or radiolabeling reaction. In some embodiments, the marker can be a gene product that interrupts or inhibits a particular function of the cells being tested.

### Pharmaceutical Compositions

An MYXV of the disclosure or a cell comprising an MYXV of the disclosure can be formulated as an ingredient in a pharmaceutical composition. Therefore, in some embodiments, the disclosure provides a pharmaceutical composition comprising a Myxoma virus expressing one or more multi-specific immune cell engagers, such as BiKE, BiTE and/or MiTE, and a pharmaceutically acceptable diluent or excipient. The compositions may contain pharmaceutically acceptable concentrations of salt, buffering agents, preservatives and various compatible carriers.

The pharmaceutical compositions may contain additional therapeutic agents, such as additional anti-cancer agents. In one embodiment, the compositions include a chemotherapeutic agent. The chemotherapeutic agent, for example, may be substantially any agent, which exhibits an effect against cancer cells or neoplastic cells of the subject and that does not inhibit or diminish the tumor killing effect of the MYXV expressing one or more multi-specific immune cell engagers. For example, the chemotherapeutic agent may be, without limitation, an anthracycline, an alkylating agent, an alkyl sulfonate, an aziridine, an ethylenimine, a methylmelamine, a nitrogen mustard, a nitrosourea, an antibiotic, an antimetabolite, a folic acid analogue, a purine analogue, a pyrimidine analogue, an enzyme, a podophyllotoxin, a platinum-containing agent or a cytokine. The chemotherapeutic agent can be one that is known to be effective against the particular cell type that is cancerous or neoplastic.

The proportion and identity of the pharmaceutically acceptable diluent can be determined, for example, by chosen route of administration, compatibility with a live virus, and standard pharmaceutical practice. In some embodiments, the pharmaceutical composition will be formulated with components that will not significantly impair the biological properties of the MYXV expressing one or more multi-specific immune cell engagers, such as BiKE, BiTE and/or MiTE. The pharmaceutical composition can be prepared by known methods for the preparation of pharmaceutically acceptable compositions suitable for administration to subjects, such that an effective quantity of the active substance or substances is combined in a mixture with a pharmaceutically acceptable vehicle. Suitable vehicles are described, for example, in Remington's Pharmaceutical Sciences (Remington's Pharmaceutical Sciences, Mack Publishing Company, Easton, Pa., USA 1995). On this basis, the compositions can comprise solutions of the MYXV or cells comprising the MYXV in association with one or more pharmaceutically acceptable vehicles or diluents, and contained in buffer solutions with a suitable pH and iso-osmotic with physiological fluids.

The pharmaceutical composition may be administered to a subject in a variety of forms depending on the selected route of administration, as disclosed herein. The composition of the disclosure may be administered orally or parenterally. Parenteral administration includes intravenous, intratumoral, intraperitoneal, subcutaneous, intramuscular, transepithelial, nasal, intrapulmonary, intrathecal, rectal and topical modes of administration. Parenteral administration may be by continuous infusion over a selected period of time (e.g., intravenous infusion).

The pharmaceutical composition may be administered orally, for example, with an inert diluent or with a carrier, or it may be enclosed in hard or soft shell gelatin capsules, or it may be compressed into tablets. For oral therapeutic administration, the MYXV expressing one or more multi-specific immune cell engagers (such as BiKE, BiTE and/or MiTE) may be incorporated with an excipient and be used in the form of ingestible tablets, buccal tablets, troches, capsules, elixirs, suspensions, syrups, wafers and the like.

Solutions of an MYXV of the disclosure or cells comprising an MYXV of the disclosure can be prepared in a physiologically suitable buffer. Under ordinary conditions of storage and use, these preparations can contain a preservative to prevent the growth of microorganisms, but that will not inactivate the live virus. Conventional procedures and ingredients for the selection and preparation of suitable formulations are described, for example, in Remington's Pharmaceutical Sciences and in The United States Pharmacopeia: The National Formulary (USP 24 NF19) published in 1999. The dose of the pharmaceutical composition that is to be used depends on the particular condition being treated, the severity of the condition, the individual subject parameters including age, physical condition, size and weight, the duration of the treatment, the nature of concurrent therapy (if any), the specific route of administration and other similar factors that are within the knowledge and expertise of the health practitioner. In certain embodiments, the therapeutic virus may be freeze dried for storage at room temperature.

### Kits

Aspects of the present disclosure concern a MYXV expressing one or more multi-specific immune cell engagers, such as BiKE, BiTE and/or MiTE, and kits including the same. The MYXV expressing one or more multi-specific immune cell engagers, such as BiKE, BiTE and/or MiTE, or pharmaceutical compositions comprising the MYXV, can be packaged as a kit, for example, containing instructions for use of the MYXV. A kit can comprise any MYXV disclosed herein, for example, a expressing one or more multi-specific immune cell engagers, such as BiKE, BiTE and/or MiTE, one or more reporter transgenes, one or more non-immunomodulatory transgenes, or a combination thereof. In some embodiments, a kit comprises a MYXV-BiTE, a MYXV-BiKE, a MYXV-MiTE, or a combination thereof. The kit can comprise one or more pharmaceutically-acceptable buffers, diluents, carriers, excipients, or vehicles, for example, for formulating the MYXV into a dosage form for administration to a recipient subject.

Disclosed herein, in some embodiments, is a kit that comprises a MYXV of the disclosure (e.g., a MYXV that expresses a multi-specific immune cell engager, such as BiKE, BiTE and/or MiTE), and materials for cellular delivery of MYXV as disclosed herein. The kit can comprise, for example, a plurality of cells, such as leukocytes from bone marrow and/or peripheral blood. The leukocytes can be autologous, allogeneic, haploidentical, HLA-matched, or HLA-mismatched relative to a subject who will be a recipient of the MYXV and the cells. In some embodiments, the plurality of cells is pre-adsorbed with or have been exposed to the MYXV of the disclosure. The kit can comprise instructions for adsorbing the MYXV to the plurality of cells and/or administering the MYXV-adsorbed cells to a recipient. The kit can comprise one or more pharmaceutically-acceptable buffers, diluents, carriers, excipients, or vehicles, for example, for adsorbing the MYXV to the plurality of cells, removing unbound MYXV, formulating the MYXV-adsorbed cells into a dosage form for administration to a recipient subject, or any combination thereof.

In some embodiments, the kit comprises a MYXV of the disclosure and a plurality of cells. In some embodiments, the kit comprises a MYXV of the disclosure and instructions for adsorbing the MYXV to the plurality of cells and/or administering the MYXV-adsorbed cells to a recipient. In some embodiments, the kit comprises a MYXV of the disclosure and one or more pharmaceutically-acceptable buffers, diluents, carriers, excipients, or vehicles. In some embodiments, the kit comprises a MYXV of the disclosure, a plurality of cells, and instructions for adsorbing the MYXV to the plurality of cells and/or administering the MYXV-adsorbed cells to a recipient. In some embodiments, the kit comprises a MYXV of the disclosure, a plurality of cells, and one or more pharmaceutically-acceptable buffers, diluents, carriers, excipients, or vehicles. In some embodiments, the kit comprises a MYXV of the disclosure, instructions for adsorbing the MYXV to the plurality of cells and/or administering the MYXV-adsorbed cells to a recipient, and one or more pharmaceutically-acceptable buffers, diluents, carriers, excipients, or vehicles. In some embodiments, the kit comprises a plurality of cells, instructions for adsorbing a MYXV to the plurality of cells and/or administering the MYXV-adsorbed cells to a recipient, and one or more pharmaceutically-acceptable buffers, diluents, carriers, excipients, or vehicles. In some embodiments, the kit comprises a MYXV of the disclosure, a plurality of cells, instructions for adsorbing the MYXV to the plurality of cells and/or administering the MYXV-adsorbed cells to a recipient, and one or more pharmaceutically-acceptable buffers, diluents, carriers, excipients, or vehicles.

### EXAMPLES

### Example 1: Design and Construction of Recombinant MYXV Construct Expressing a BiKE

This example demonstrates the design and construction of a myxoma virus that expresses a Bi-specific Natural Killer and Neutrophil engager (BiKE). BiKEs can contain one domain that specifically binds to an antigen expressed on the surface of NK cells and/or neutrophils (e.g., CD16), and one domain that specifically binds to an antigen expressed on a target cell (e.g., CD138 for multiple myeloma cells). These molecules are designed to form an antigen-specific immunological synapse between NK cells or neutrophils and tumor cells in order to trigger enhanced NK/neutrophil cell-mediated killing of tumor targets. Expressing a BiKE construct (e.g., a secreted construct) from MYXV could potentiate the capacity of NK and neutrophils to kill MM cells in the tumor microenvironment, and the virus-expressed BiKE technology could also be applied to any other cancers for which a cancer-specific cell surface marker exists.

A BiKE was designed comprising two single chain variable fragments (scFvs) derived from antibodies, joined by a short peptide linker. One scFv arm binds CD16 on the surface of NK cells and neutrophils, while the other binds a chosen target antigen (in this case CD138, a hallmark of multiple myeloma (MM) cells).

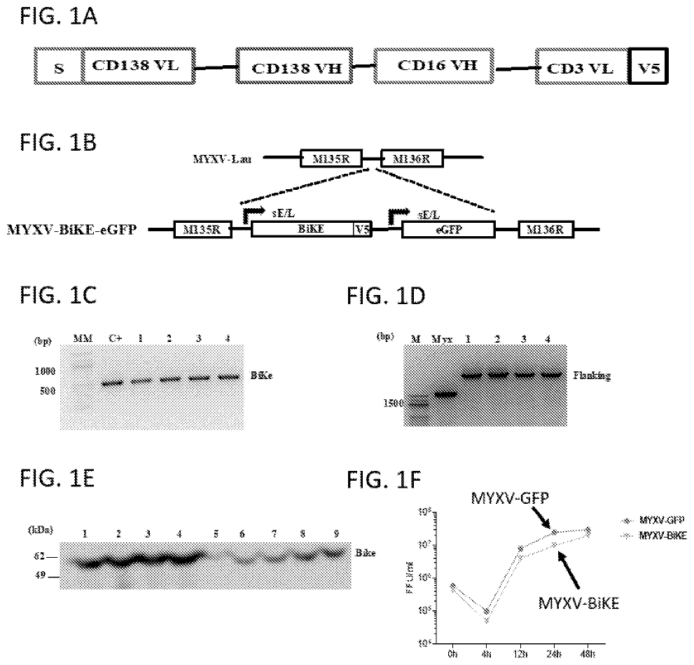

The anti-CD16 scFv human Ab sequences (heavy and light chain variable domains) were obtained from publicly available sources (AY345160.1 and AY345161.2; Genbank). The anti-CD138 scFv sequence was obtained from a publicly available source (Genbank). To form scFvs, the anti-CD16 variable regions were connected by a (G4S1)3 linker, and the anti-CD138 variable regions were connected by a (G4S1)3 linker. The anti-CD16 and anti-CD138 scFvs were connected to each other by a (G4S1)2 flexible linker. The BiKE was arranged, from N-to-C terminus, VL(CD138)-VH(CD138)-VH(CD16)-VL(CD16), and included the signal peptide from the mouse Ig heavy chain at the N-terminus, and a V5 tag at the C-terminus (FIG. 1A and SEQ ID NO: 6). The BiKE construct was optimized for human codon usage and synthesized by Genscript.

MYXV-BiKE-GFP was constructed by inserting a BiKE-expressing cassette containing the BiKE coding sequence (GenScript) with a C-terminal V5-tag and under the control of the poxvirus synthetic early/late promoter (sE/L) at an intergenic location between the M135 and M136 genes in the wild-type (wt) MYXV strain Laussane (MYXV-Lau) genome. An expression cassette for an enhanced green fluorescent protein (eGFP) was inserted immediately downstream of the BiKE expression cassette, and its expression was also driven by a poxvirus synthetic early/late promoter (FIG. 1B). The eGFP can serve as a fluorescent marker for MYXV replication in vitro and in vivo, as MYXV infection can be monitored by live imaging of GFP expression.

To create the MYXV-BiKE construct, a recombinant plasmid was first constructed using Gateway System (ThermoFisher Scientific). Upstream and downstream hybridizing sequences were amplified by PCR to generate entry clones by Gateway BP recombination with appropriate pDONR vectors. The final recombinant plasmid was constructed by recombining three entry clones with a destination vector in a sequential manner. The BiKE and eGFP expression cassettes were inserted into the MYXV genome by infecting RK13 cells with MYXV-Lau and then transfecting the appropriate recombination plasmid. Multiple rounds of foci purification were conducted to obtain pure stocks of the recombinant viruses, the specificity confirmed by PCR using the appropriate primers set:

BIKE_CD16_F
(SEQ ID NO: 1)
TCAGCAAGGACACATCCTCTAA
BIKE_CD16_R
(SEQ ID NO: 2)
TAAGGATCCTCATTGGACTGC

The purity was also confirmed by PCR using the appropriate primers set (FIGS. 1C-D).

BiKE expression was confirmed by Western Blot, by detecting the presence of a band of 56 kDa in lysates from MYXV-BiKE infected RK13 cells by using a mouse monoclonal Ab specific to the V5 tag (Invitrogen), both in cell lysates and supernatants (FIG. 1E). The replication capacity of the new construct MYXV-BiKE in RK13 cells was similar to the parental virus MYXV-GFP (FIG. 1F).

SEQ ID NO: 3 provides the nucleotide sequence of a transgene encoding the BiKE. SEQ ID NO: 4 provides the amino acid sequence of a transgene encoding the BiKE. In SEQ ID NO: 4, the N-terminal signal sequence is underlined, linkers are in bold, and the C-terminal V5 tag is in italics. In some embodiments, a mature form of BiKE does not comprise the signal sequence and/or V5 tag. For example, in some embodiments a mature BiKE of the disclosure comprises the sequence of SEQ ID NO: 5.

DNA BiKE sequence:
(SEQ ID NO: 3)
ATGAAGAGCCAGACCCAGGTGTTCATCTTCCTGCTGCTGTGCGTGAGCG
GCGCCCACGGCGACATCCAGATGACCCAGAGCACCAGCAGCCTGAGCGC
CAGCCTGGGCGACAGGGTGACCATCAGCTGCAGCGCCAGCCAGGGCATC
AACAACTACCTGAACTGGTACCAGCAGAAGCCCGACGGCACCGTGGAGC
TGCTGATCTACTACACCAGCACCCTGCAGAGCGGCGTGCCCAGCAGGTT
CAGCGGCAGCGGCAGCGGCACCGACTACAGCCTGACCATCAGCAACCTG
GAGCCCGAGGACATCGGCACCTACTACTGCCAGCAGTACAGCAAGCTGC
CCAGGACCTTCGGCGGCGGCACCAAGCTGGAGATCAAGGGTGGCGGTGG
CTCCGGCGGTGGTGGGTCGGGTGGCGGCGGATCTAGCCAGGTGCAGCTG
CAGCAGAGCGGCAGCGAGCTGATGATGCCCGGCGCCAGCGTGAAGATCA
GCTGCAAGGCCACCGGCTACACCTTCAGCAACTACTGGATCGAGTGGGT
GAAGCAGAGGCCCGGCCACGGCCTGGAGTGGATCGGCGAGATCCTGCCC
GGCACCGGCAGGACCATCTACAACGAGAAGTTCAAGGGCAAGGCCACCT
TCACCGCCGACATCAGCAGCAACACCGTGCAGATGCAGCTGAGCAGCCT
GACCAGCGAGGACAGCGCCGTGTACTACTGCGCCAGGAGGGACTACTAC
GGCAACTTCTACTACGCCATGGACTACTGGGGCCAGGGCACCAGCGTGA
CCGTGAGCAGCGGTGGCGGTGGCTCCGGCGGTGGTGGGTCGCAGGTTAC
TCTGAAAGAGTCTGGCCCTGGGATATTGCAGCCCTCCCAGACCCTCAGT
CTGACTTGTTCTTTCTCTGGGTTTTCACTGAGGACTTCTGGTATGGGTG
TAGGCTGGATTCGTCAGCCTTCAGGGAAGGGTCTAGAGTGGCTGGCACA
CATTTGGTGGGATGATGACAAGCGCTATAATCCAGCCCTGAAGAGCCGA
CTGACAATCTCCAAGGATACCTCCAGCAACCAGGTATTCCTCAAAATCG
CCAGTGTGGACACTGCAGATACTGCCACATACTACTGTGCTCAAATAAA
CCCCGCCTGGTTTGCTTACTGGGGCCAAGGGACTCTGGTCACTGTCTCT
GCCGGTGGCGGTGGCTCCGGCGGTGGTGGGTCGGGTGGCGGCGGATCTG
ACACTGTGCTGACCCAATCTCCAGCTTCTTTGGCTGTGTCTCTAGGGCA
GAGGGCCACCATCTCCTGCAAGGCCAGCCAAAGTGTTGATTTTGATGGT
GATAGTTTTATGAACTGGTACCAACAGAAACCAGGACAGCCACCCAAAC
TCCTCATCTATACTACATCCAATCTAGAATCTGGGATCCCAGCCAGGTT
TAGTGCCAGTGGGTCTGGGACAGACTTCACCCTCAACATCCATCCTGTG
GAGGAGGAGGATACTGCAACCTATTACTGTCAGCAAAGTAATGAGGATC
CGTACACGTTCGGAGGGGGGACCAAGCTGGAAATAAAAGGTAAGCCTAT
CCCTAACCCTCTCCTCGGTCTCGATTCTACGTAA.
Protein BiKE sequence:
(SEQ ID NO: 4)
MKSQTQVFIFLLLCVSGAHGDIQMTQSTSSLSASLGDRVTISCSASQGI
NNYLNWYQQKPDGTVELLIYYTSTLQSGVPSRFSGSGSGTDYSLTISNL
EPEDIGTYYCQQYSKLPRTFGGGTKLEIKGGGGSGGGGSGGGGSSQVQL
QQSGSELMMPGASVKISCKATGYTFSNYWIEWVKQRPGHGLEWIGEILP
GTGRTIYNEKFKGKATFTADISSNTVQMQLSSLTSEDSAVYYCARRDYY
GNFYYAMDYWGQGTSVTVSSGGGGSGGGGSQVTLKESGPGILQPSQTLS
LTCSFSGFSLRTSGMGVGWIRQPSGKGLEWLAHIWWDDDKRYNPALKSR
LTISKDTSSNQVFLKIASVDTADTATYYCAQINPAWFAYWGQGTLVTVS
AGGGGSGGGGSGGGGSDTVLTQSPASLAVSLGQRATISCKASQSVDFDG
DSFMNWYQQKPGQPPKLLIYTTSNLESGIPARFSASGSGTDFTLNIHPV
EEEDTATYYCQQSNEDPYTFGGGTKLEIKGKPIPNPLLGLDST.
Protein BiKE sequence lacking signal peptide and
V5 tag:
(SEQ ID NO: 5)
DIQMTQSTSSLSASLGDRVTISCSASQGINNYLNWYQQKPDGTVELLIY
YTSTLQSGVPSRFSGSGSGTDYSLTISNLEPEDIGTYYCQQYSKLPRTF
GGGTKLEIKGGGGSGGGGSGGGGSSQVQLQQSGSELMMPGASVKISCKA
TGYTFSNYWIEWVKQRPGHGLEWIGEILPGTGRTIYNEKFKGKATFTAD
ISSNTVQMQLSSLTSEDSAVYYCARRDYYGNFYYAMDYWGQGTSVTVSS
GGGGSGGGGSQVTLKESGPGILQPSQTLSLTCSFSGFSLRTSGMGVGWI
RQPSGKGLEWLAHIWWDDDKRYNPALKSRLTISKDTSSNQVFLKIASVD
TADTATYYCAQINPAWFAYWGQGTLVTVSAGGGGSGGGGSGGGGSDTVL
TQSPASLAVSLGQRATISCKASQSVDFDGDSFMNWYQQKPGQPPKLLIY
TTSNLESGIPARFSASGSGTDFTLNIHPVEEEDTATYYCQQSNEDPYTF
GGGTKLEIK.

### Example 2: In Vitro Studies Using Human Primary Patient Samples Contaminated with Multiple Myeloma (MM) Cells

In order to evaluate the susceptibility of primary patient samples contaminated with multiple myeloma (MM) cells from drug-refractory patients to MYXV infection, primary un-manipulated peripheral blood (PB) from patients three patients with different levels of MM cells (CD138+) were subjected to purification using Ficoll-paque plus gradient to isolate mononuclear cells and eliminate the majority of red blood cells (RBCs). The patients are referred to as patient #s 2, 3, and 4.

TABLE 4
Percentages of primary MM cells (CD138+)

| Patient # | % MM cells |
|---|---|
| 2 | <1.0 |
| 3 | 2.3 |
| 4 | 15.0 |

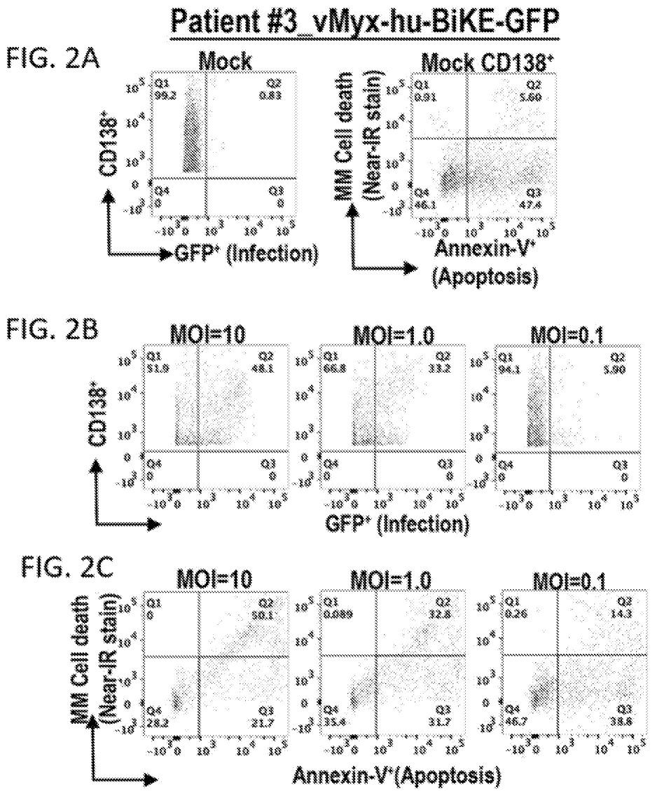

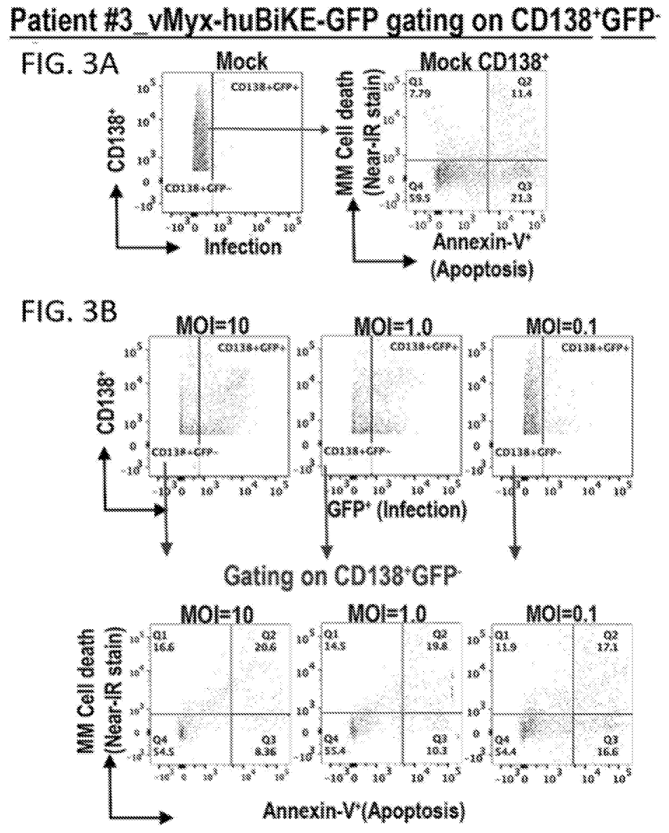

These primary cells in suspension were then mock-treated (i.e., no virus added), or incubated with MYXV-BiKE-GFP at 37° C. for 1 hour to allow virus adsorption. Experiments were conducted at different multiplicities of infection (MOI) including MOI=10, 1, and 0.1 as shown in FIGS. 2-5 and Tables 4-5. After this, mock-treated, or MYXV-treated cells were incubated overnight (˜24 hours) at 37° C. to allow virus infection. For patient #3, the percentages of virus infection (i.e., using MYXV-BiKE) at MOI=10, 1.0 and 0.1, as well as percentages of viability, apoptosis, and cell death of MM cells were determined using flow cytometry (FIGS. 2A-C). In addition to this, the percentages of viability, apoptosis, and cell death of uninfected MM cells in patient samples that were exposed to the virus were evaluated using flow cytometry (FIGS. 3A-B). This allows the measurement of MM cell death in cells that were not directly infected by the virus (e.g., do not express any virus-specific fluorescent protein), but were killed in an “off-target” fashion, e.g., by MYXV-activated or BiKE-activated leukocytes from the same patient samples.

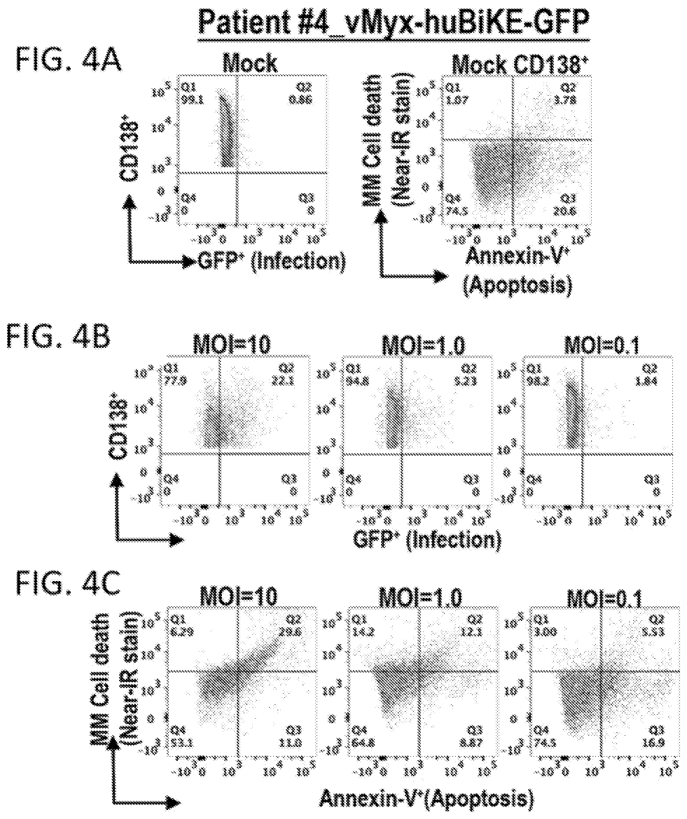

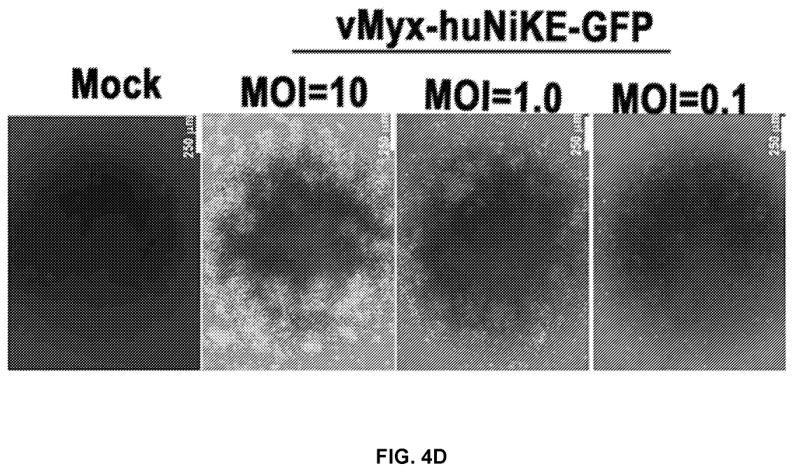

The levels of infection using MYXV-BiKE-GFP in cells from patient #4 were first evaluated using fluorescence microscopy (FIG. 4D). The percentages of infected, viable, and apoptotic MM cells were determined using flow cytometry (FIG. 4A-C). Furthermore, the percentages of viability, apoptosis, and cell death of those myeloma cells that were exposed to the virus but were un-infected was evaluated using flow cytometry (FIGS. 5A-B).

CD138 was used as a marker of multiple myeloma (MM) cells. GFP was used as a marker of MYXV-infected cells. Annexin-V was used as a marker of apoptotic cells. Near-IR stain was used as a marker of dead cells.

Tables 4 & 5 summarize the data from patients #3 and #4. Because the amount of CD138⁺ MM cells in patient 2 was below 1%, the percentages of MM cell infection and cell death could not be determined for that patient. The apoptosis and MM cell killing data shown in Table 5 was generated by gating on CD138⁺ (MM) cells, and data indicate that MYXV-BiKE can efficiently infect and kill MM cells within primary human peripheral blood samples derived from drug-refractory patients. For patient #3 the MYXV-BiKE increased killing of MM cells at all the different MOI's tested (FIGS. 2A-C, and Table 5). For example, 50.1% of cells infected with MYXV expressing huBiKE at an MOI of 10 were killed, and 6.51% of mock-treated cells were killed. For patient #4, MYXV-BiKE also killed infected MM cells following virus infection (FIGS. 4A-C and Table 5). For example, 35.9% of cells infected with MYXV expressing BiKE at an MOI of 10 were killed, and 4.85% of mock-treated cells were killed.

TABLE 5
Percentages of infection, apoptosis, and cell death of hu-primary
MM cells (CD138⁺) 24 hours after exposure to MYXV-BIKE.

| | Patient #3 | | | | Patient #4 | | | |
|---|---|---|---|---|---|---|---|---|
| | | MOI = | MOI = | MOI = | | MOI = | MOI = | MOI = |
| | Mock | 10 | 1 | 0.1 | Mock | 10 | 1 | 0.1 |
| % infection | 0.83 | 48.1 | 33.2 | 5.9 | 0.86 | 22.1 | 5.23 | 1.84 |
| % Annexin V− | 0.91 | 0 | 0.087 | 0.26 | 1.07 | 6.29 | 14.2 | 3 |
| IR+ | | | | | | | | |
| % Annexin V+ | 5.6 | 50.1 | 32.8 | 14.3 | 3.78 | 29.6 | 12.1 | 5.53 |
| Near-IR+ | | | | | | | | |
| % Near-IR+ | 6.51 | 50.1 | 32.9 | 14.56 | 4.85 | 35.9 | 26.3 | 8.53 |

MOI = multiplicity of infection.
Mock = mock infected.
% infection as determined by GFP positivity.
Annexin V+ indicates Annexin V positive (apoptotic or dead).
Annexin V− indicates Annexin V negative (not apoptotic or dead).
Near-IR+ indicates Near IR+ (dead).

Data shown in Table 6 was generated by gating on killing of uninfected MM cells (i.e., CD138⁺GFP⁻). This “off-target” killing of un-infected MM cells was higher for MYXV-BiKE at all MOIs compared to mock-treated cells (FIGS. 3A-B and Table 6 for patient #3, and FIGS. 5A-B and Table 6 for patient #4). For example, 96.12% of uninfected cells from patient 4 were killed in experiments where MYXV-BiKE had been added to the culture at an MOI of 10, compared to 5.41% of mock-treated cells.

TABLE 6
Percentages of viability, apoptosis, and cell death of uninfected (GFP negative)
hu-primary MM cells (CD138⁺) 24 hours after culture exposure to MYXV-BIKE.

| | Patient #3 | | | | | Patient #4 | | | | |
|---|---|---|---|---|---|---|---|---|---|---|
| | | MOI = | MOI = | MOI = | | | MOI = | MOI = | MOI = | |
| | Mock | 10 | 1 | 0.1 | | Mock | 10 | 1 | 0.1 | |
| % Annexin V− | | 7.79 | | 16.6 | 14.5 | 11.9 | 1.11 | 9.32 | 24.7 | 26.1 |
| IR+ | | | | | | | | | | |
| % Annexin V+ | | 11.4 | 20.6 | 19.7 | 17.1 | 4.3 | 86.8 | 60.1 | 31.9 | |
| Near-IR+ | | | | | | | | | | |

| | | | | | | | | |
|---|---|---|---|---|---|---|---|---|
| % Near-IR+ | 19.2 | 37.2 | 34.3 | 29 | 5.41 | 96.12 | 84.8 | 58 |

MOI = multiplicity of infection.
Mock = mock infected.
Annexin V+ indicates Annexin V positive (apoptotic or dead).
Annexin V− indicates Annexin V negative (not apoptotic or dead).
Near-IR+ indicates Near IR+ (dead).

### Example 3: BiKE Specifically Binds to Human Multiple Myeloma Cells and Human Natural Killer Cells

This example demonstrates that a BiKE of the disclosure specifically binds to human multiple myeloma (MM) cells and human natural killer (NK) cells.

The MYXV-BiKE described in Example 1 was propagated in RK13 cells. Supernatants from MYXV-BiKE-infected RK13 cells, containing secreted BiKE, were harvested. As controls, supernatants were also harvested from RK13 cells that were mock infected, or infected with wild type MYXV.

Harvested supernatants were added to cultures of human NK cells or human MM (U266) cells. To detect BiKE bound to cells, cells were stained with a PE-conjugated monoclonal antibody specific for the V5 tag at the C-terminus of BiKE, washed, and analyzed by flow cytometry.

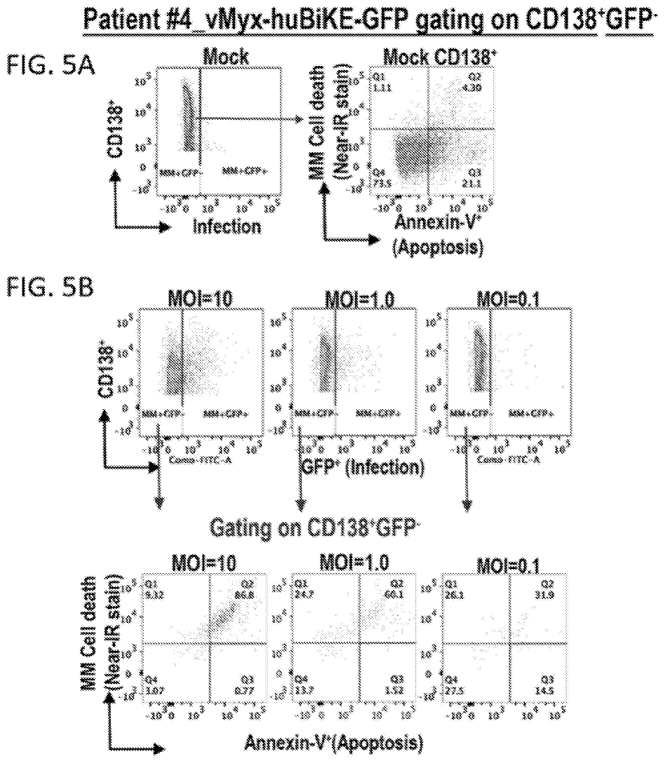

FIG. 6 demonstrates that BiKE bound to human MM and NK cells, while binding was not detected for control MM cells or NK cells (incubated with supernatants harvested from mock-infected or wild type-MYXV-infected cells).

### Example 4: BiKE Increases Killing of Human Multiple Myeloma Cells Co-Cultured with Human Natural Killer Cells

This example demonstrates that a BiKE of the disclosure increases killing of human multiple myeloma (MM) cells co-cultured with human natural killer (NK) cells.

RK13 cells were infected with the MYXV-BiKE described in Example 1 at multiplicities of infection (MOIs) of 1, 5, or 10. Supernatants from the MYXV-BiKE-infected RK13 cells, containing secreted BiKE, were harvested. As controls, supernatants were also harvested from RK13 cells that were mock infected.

Harvested supernatants were added to co-cultures containing primary human NK cells and human MM (U266) cells. After incubation for 24 hours, cells were stained to identify MM cells (CD138+) and dead cells (Near IR stain), then analyzed by flow cytometry.

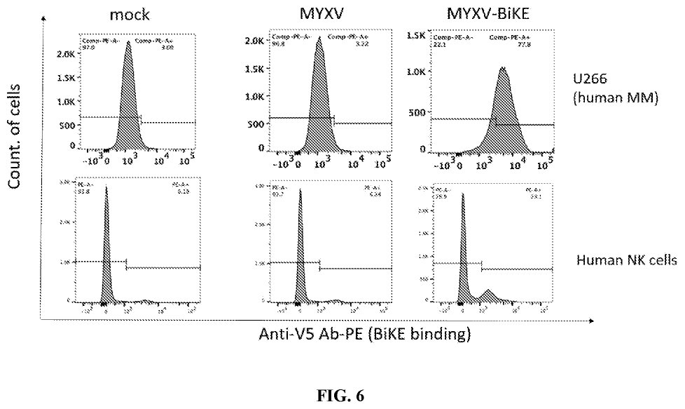

FIG. 7 demonstrates that the BiKE antibodies were able to induce NK-cell-mediated killing of MM cells, and that killing was dependent on the MOI of the source supernatant culture.

For co-cultures incubated in supernatants from mock-infected cells, 10.6% of cells were CD138+ Near IR−, and 0.74% of cells were CD1388+, Near IR+. For co-cultures incubated in supernatants from cells infected with MYXV-BIKE at an MOI of 1, 5.17% of cells were CD138+ Near IR−, and 1.71% of cells were CD138+, Near IR+. For co-cultures incubated in supernatants from cells infected with MYXV-BIKE at an MOI of 5, 2.99% of cells were CD138+ Near IR−, and 4.42% of cells were CD1388+, Near IR+. For co-cultures incubated in supernatants from cells infected with MYXV-BIKE at an MOI of 10, 0.024% of cells were CD138+ Near IR−, and 7.09% of cells were CD1388+, Near IR+.

A larger co-culture experiment was performed using BiKE harvested from Vero cells, with a comparison of NK effector cells to NK-depleted PBMC effector cells, and a 48 hour incubation with BiKE.

PBMCs from primary human peripheral blood from healthy patients were first isolated using Ficoll-Paque PLUS gradient. NK cells were isolated from these PBMCs using MACS human NK cell isolation kit and LS magnetic columns to deplete magnetically labeled cells. The NK cells or PBMCs were then co-incubated with human MM target cells (U266 cells), in the presence or absence of BiKE, and the effect of BiKE on viability determined. 1×10\hat{ }6 NK cells or PBMCs were incubated with 2×10\hat{ }5 U266 cells in the presence of either complete media, 0.5×MYXV-GFP supernatant (250 μL complete media+250 μL serum-free RPMI supernatant from Vero cells infected with MYXV-GFP at MOI 5 for 48 hours), 0.25×MYXV-BIKE-GFP supernatant (375 μL complete media+125 μL serum-free RPMI supernatant from Vero cells infected with MYXV-BIKE-GFP at MOI 5 for 48 hours), or 0.5×MYXV-BIKE-GFP sup (prepared similarly). All samples were incubated in 24-well plates at 37° C. At 48 hours post treatment, the cells were then labeled with near-IR LIVE/DEAD stain. The cells were subsequently labeled with 1 μL human anti-CD138 antibody (to identify MM cells) and 1 μL anti-V5 antibody (to detect the V5 tag on the BIKE construct) in 100 μL staining buffer per condition and incubated for 15 minutes at 4° C., protected from light. All samples were then fixed using 100 μL Cytofix and then incubated for 15 minutes at 4° C. (protected from light) before resuspending in 270 μL staining buffer for flow cytometry analysis.

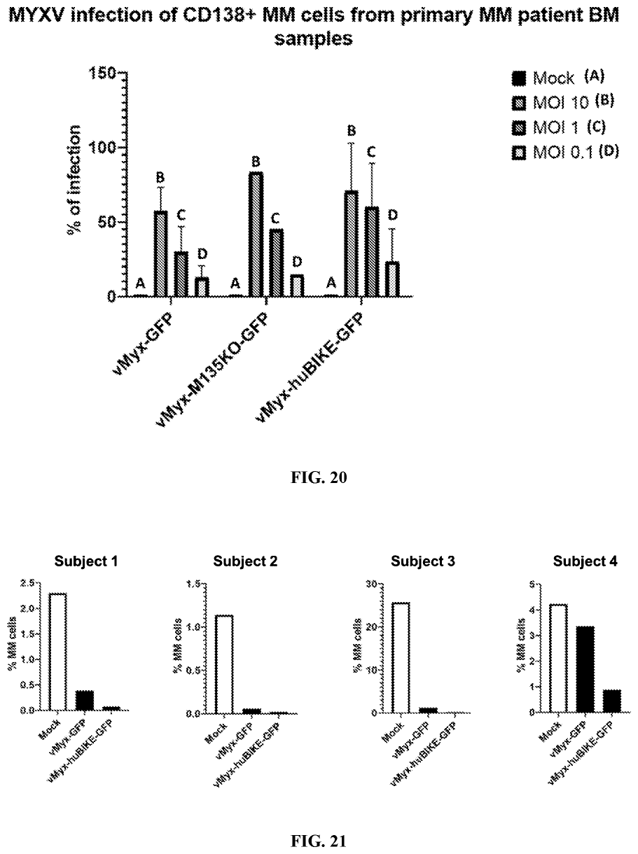

FIG. 22 shows the percent of CD138+ cells that were dead at 48 hours post-treatment. Co-cultures were performed in triplicate, and p values were obtained for each infection based on flow cytometric analysis of the proportion of the U266 cell population that were dead according to near-IR LIVE/DEAD stain. Significance (*=p<0.05; **=p<0.01; ***=p<0.001) was determined using Holm-Sidak's t test for multiple comparisons. The data show that BiKE can enhance killing of MM cells by NK cells. For example, killing of MM cells in co-cultures with NK cells was significantly higher for the samples incubated with 0.5×MYXV-BiKE supernatant than cultures incubated with 0.5×MYXV-GFP supernatant.

### Example 5: MYXV-BiKE Infects and Kills Human Hematologic Cancer Cells In Vitro

In order to evaluate the susceptibility of human hematologic cancer cells to MYXV-BiKE, human acute myeloid leukemia (AML) and multiple myeloma (MM) cell lines were infected with MYXV-BiKE. THP-1 cells were used as an example of AML cells. U266 cells were used as an example of MM cells. U266 cells were maintained in RPMI 1640 supplemented with 20% fetal bovine serum (FBS), 2 mM L-Glutamine, and 100 U/ml of penicillin-streptomycin. THP-1 cells were maintained in RPMI 1640 supplemented with 10% FBS, 2 mM L-Glutamine, and 100 U/ml of penicillin-streptomycin.

Cells were mock-infected, or infected with MYXV-BiKE-GFP, MYXV-M135KO-GFP, or wild type (WT) MYXV-GFP at a multiplicity of infection (MOI) of 0.1, 1, or 10. Cells were infected at 37° C. for 1 hour to allow virus adsorption, then incubated to 24 or 48 hours post infection (hpi).

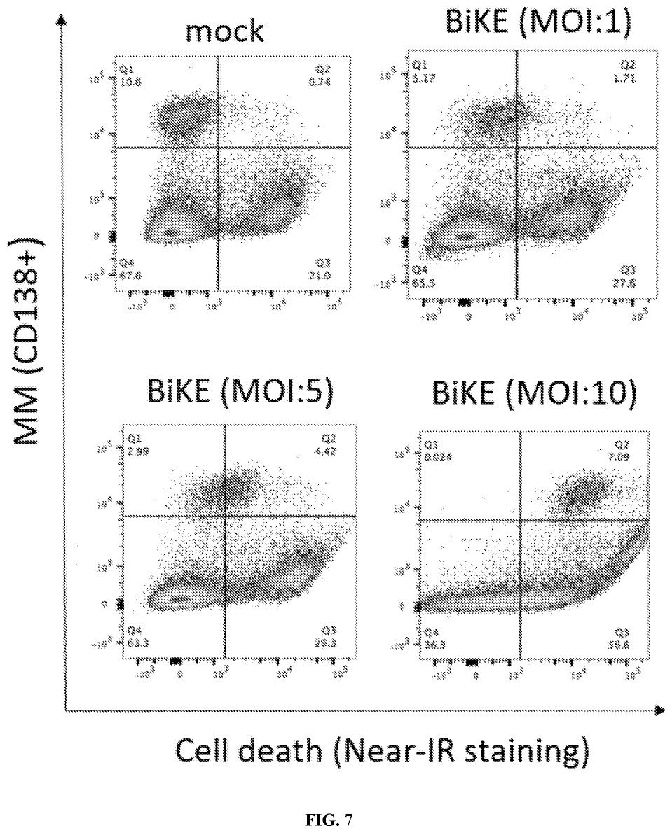

Infection was evaluated at 24 and 48 hpi by fluorescence microscopy. Images were taken at 5× magnification, with 338.00 ms exposure, and 2.5 gain. FIG. 8A and FIG. 8B demonstrate infection of THP-1 cells at 24 and 48 hours post-infection, respectively. FIG. 8C and FIG. 8D demonstrate infection of U266 cells at 24 and 48 hours post-infection, respectively.

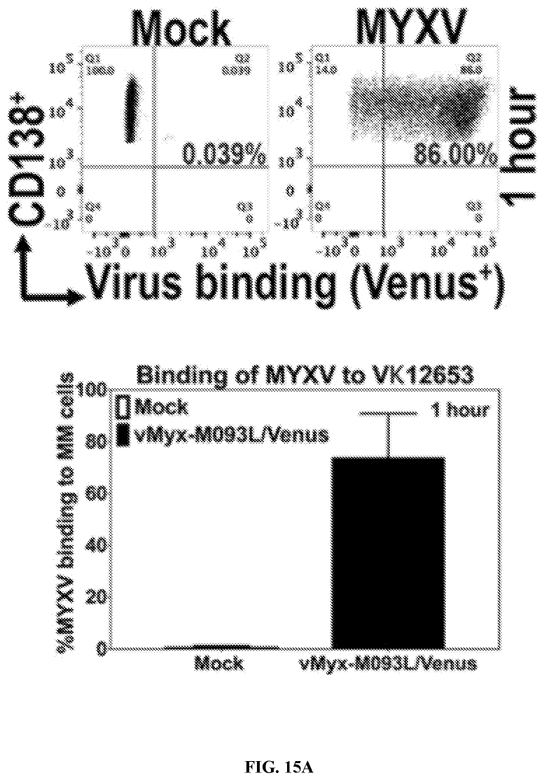

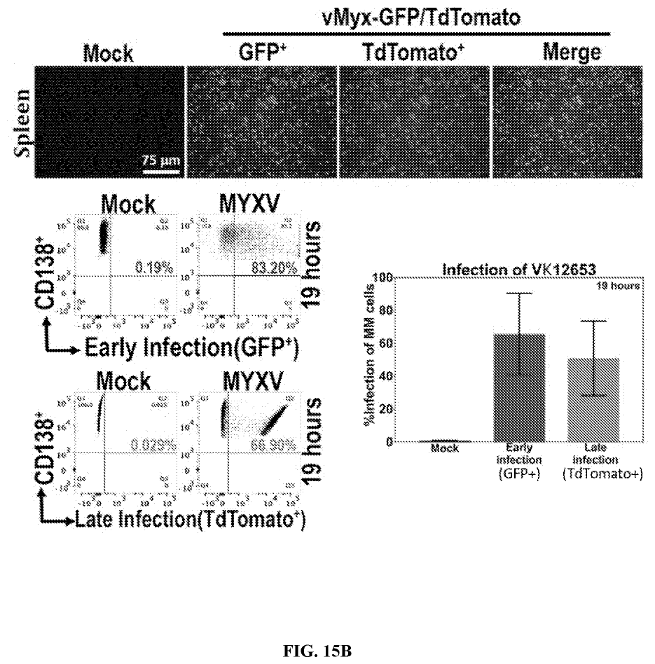

The infection rate was also quantified by flow cytometry, with populations of infected cells evaluated for GFP expression. FIG. 17A shows the percent of THP-1 cells that were GFP positive at 24 and 48 hours post-infection. FIG. 17B shows the percent of U266 cells that were GFP positive at 24 and 48 hours post-infection.

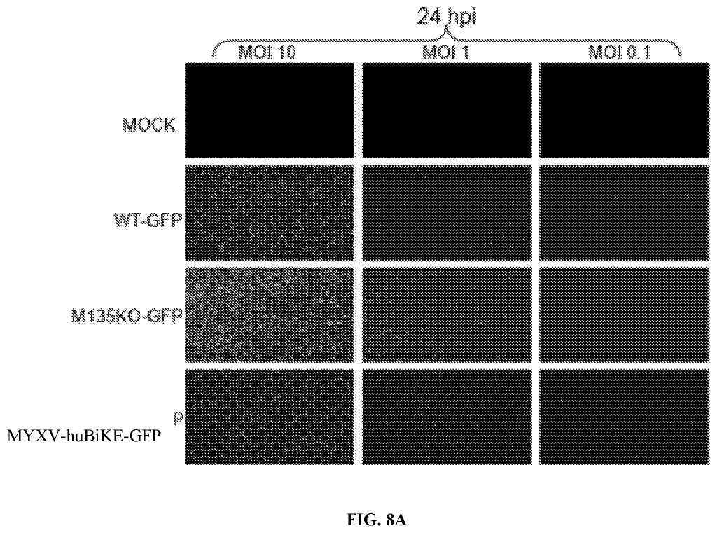

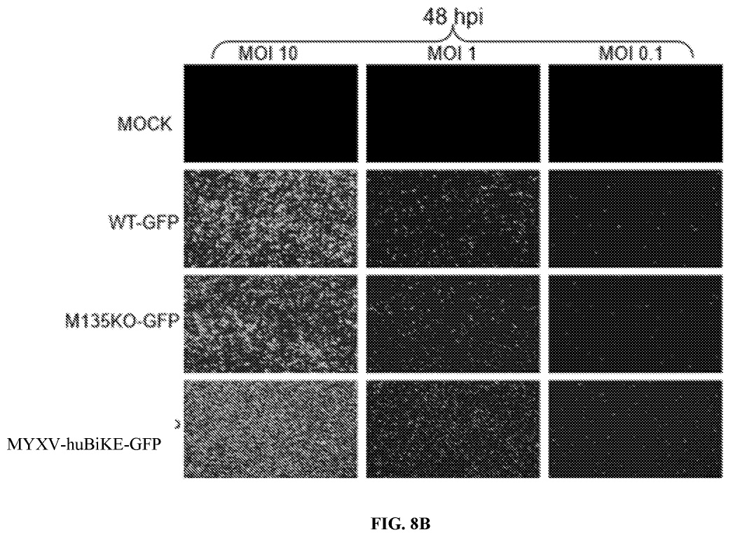

Cell killing was evaluated at 24 and 48 hours post-infection by flow cytometry using a near-IR live/dead stain. FIG. 9 demonstrates killing of THP-1 cells. FIG. 10 demonstrates killing of U266 cells.

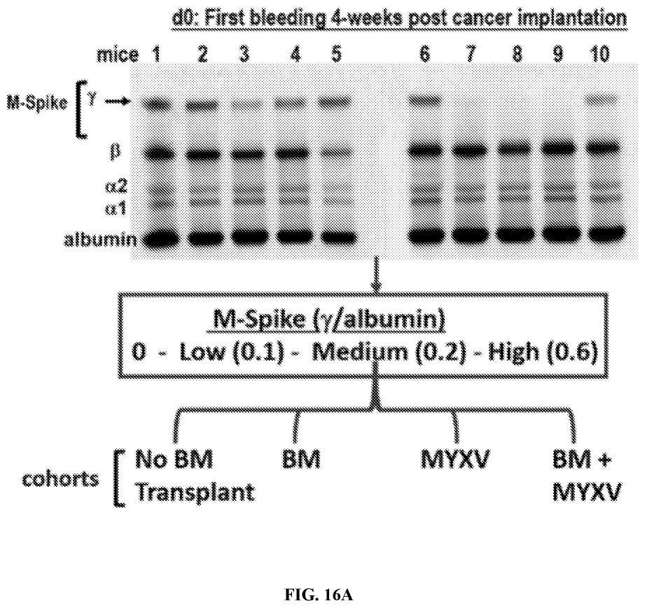

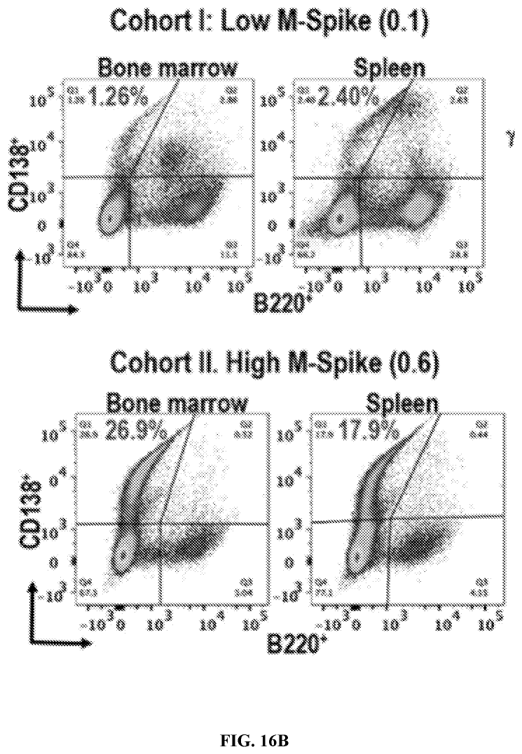

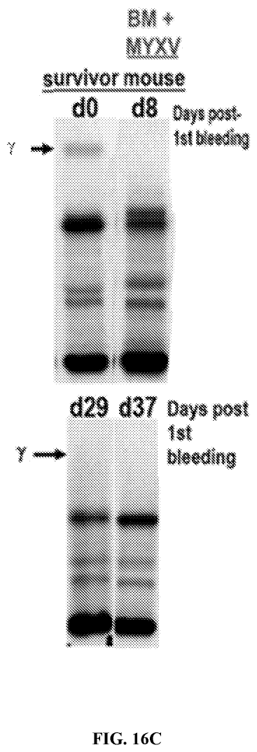

Cell killing was further characterized by gating GFP+ cells (for direct killing of infected cells, or on-target killing) and GFP negative cells (for indirect killing of uninfected cells, or off-target killing). FIG. 18A illustrates the percent of infected U266 cells that were killed at 24 and 48 hours. FIG. 18B illustrates the percent of uninfected U266 cells that were killed at 24 and 48 hours. FIG. 19 provides the ratio of dead U266 cells to infected U266 cells.

These data demonstrate that MYXV-BiKE can infect, replicate within, and kill human hematologic cancer cells, and, in some cases, can elicit enhanced killing compared to MYXV lacking BiKE. Without wishing to be bound by theory, killing induced by MYXV-BiKE may be further enhanced in conditions where effector immune cells are present that can be engaged by the BiKE construct, e.g., as demonstrated in Example 4.

### Example 6: MYXV-BiKE Kills Primary Human Multiple Myeloma Cells from Bone Marrow

This example demonstrates MYXV-BiKE killing of multiple myeloma (MM) cells in bone marrow samples from human patients.

Primary bone marrow samples were obtained from multiple myeloma patients via bone marrow biopsy, and were subjected to purification using Ficoll-paque plus gradient to isolate mononuclear cells. Mononuclear cells were then resuspended in 380 μL complete media per condition into 24-well plates.

These primary cells in suspension were then mock-treated (i.e., no virus added), or incubated with MYXV-BiKE-GFP, MYXV-M135KO-GFP, or wild type MYXV-GFP at 37° C. for 1 hour to allow virus adsorption. Experiments were conducted at different multiplicities of infection (MOI) including MOI=10, 1, and 0.1. After the 1 hour of incubation, 120 μL complete media was added to each well and the plates were incubated overnight (˜24 hours) at 37° C. to allow virus infection to progress.

At 24 hours post infection, the cells were then labeled with near-IR LIVE/DEAD stain. The primary cells were subsequently labeled with 1 μL human anti-CD138 antibody in 100 μL staining buffer per condition and incubated for 15 minutes at 4° C. with light protection. All samples were then fixed using 100 μL Cytofix and then incubated for 15 minutes at 4° C. with light protection before resuspending in 270 μL staining buffer for flow cytometry analysis.

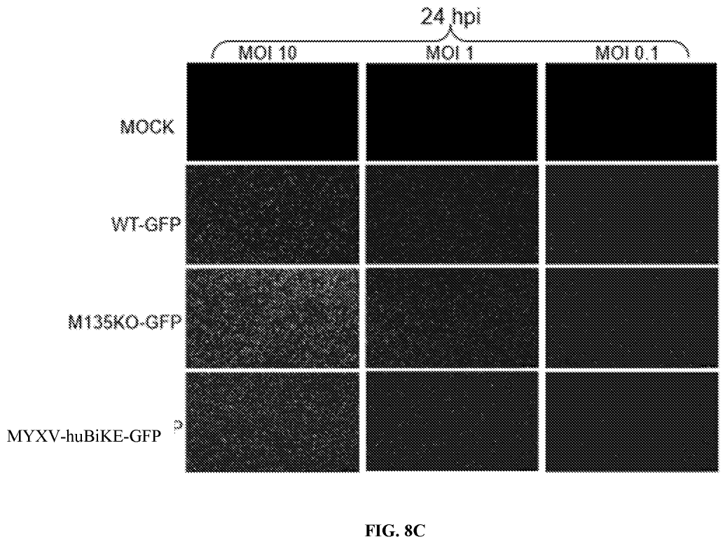

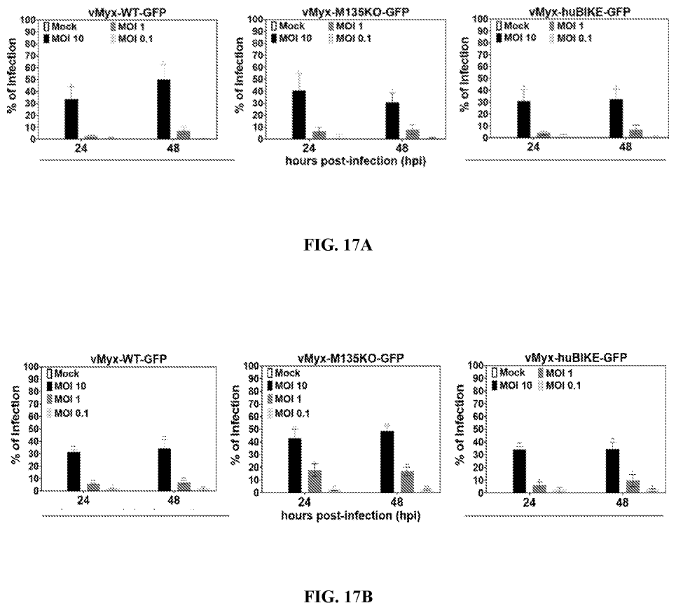

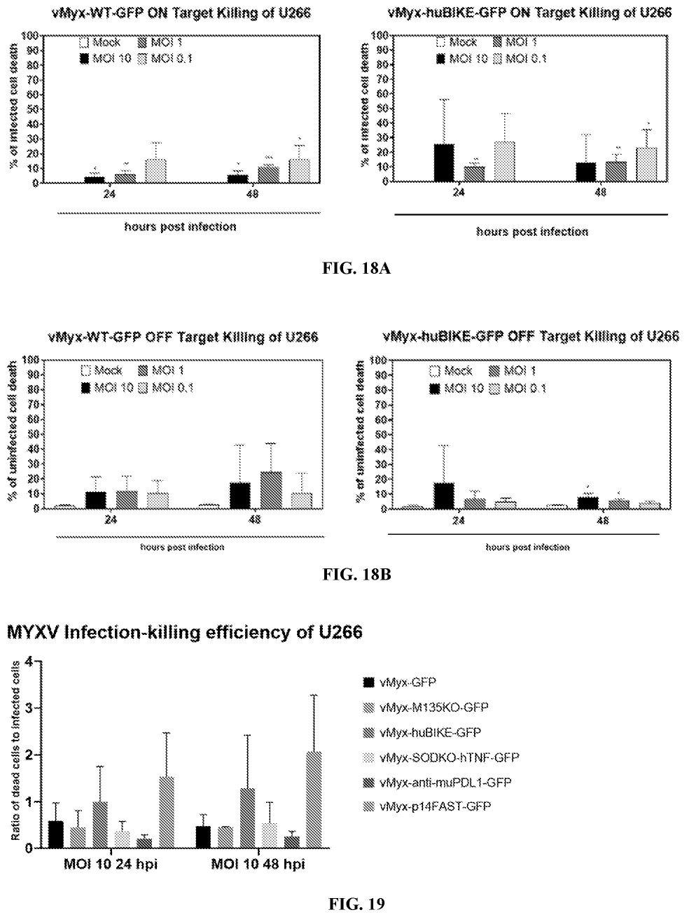

The percent of killed MM cells was evaluated by flow cytometry. CD138 was used as a marker of multiple myeloma (MM) cells. FIG. 20 illustrates the proportion of CD138+ MM cells that were infected by MYXV-BiKE-GFP, MYXV-M135KO-GFP, or wild type MYXV-GFP at the indicated MOI. FIG. 11 demonstrates killing of MM cells by MYXV-BiKE. FIG. 21 quantifies the proportion of intact cells that were CD138+ after mock-infection or infection with MYXV-BiKE-GFP or wild type MYXV-GFP at an MOI of 10, for samples obtained from four subjects.

These data demonstrate that MYXV-BiKE can infect and kill primary human hematologic cancer cells. In some cases, MYXV-BiKE is observed to elicit enhanced killing compared to a MYXV that does not express BiKE.

### Example 7: Design and Construction of Recombinant MYXV Constructs Expressing a Bispecific T Cell Engager (BiTE), Bispecific Natural Killer and Neutrophil Engager (BiKE), and/or Membrane-Integrated T Cell Engager (MiTE)

This example demonstrates the design and construction of recombinant MYXV constructs that express one or more multi-specific immune cell engagers (for example, a bispecific T cell engager (BiTE), bispecific natural killer and neutrophil engager (BiKE), and/or membrane-integrated T cell engager (MiTE)).

A DNA sequence is generated that encodes a multi-specific protein with binding specificity for an immune cell, and a binding specificity for a target antigen. For example, a single chain variable fragment (scFv) comprising the light chain variable domain and heavy chain variable domain from an antibody that binds to CD138 or CD3 can be used to confer binding specificity to an immune cell. A scFv comprising the light chain variable domain and heavy chain variable domain from an antibody that binds to CD138, CD19, EpCAM, Her2/neu, EGFR, CEA, EpHA2, CD33 or MCSP can be used to confer binding specificity to a target antigen, e.g., a target antigen expressed by a cancer cell of interest. Peptide linkers can optionally be used to link the heavy chain and light chain variable fragments of each scFv, and to link the scFvs together to form the multi-specific protein. In some cases, the protein can comprise a signal sequence to promote secretion of the multi-specific immune cell engager. In some cases, the protein can comprise a transmembrane domain for anchoring in the plasma membrane. In some cases, the protein can comprise an epitope tag for detection and/or purification of the protein (e.g., a V5 tag).

Plasmids are generated for integration of the multi-specific immune cell engager into the myxoma virus genome. The multi-specific immune cell engager (e.g., BiTE, BiKE, and/or MiTE) can be expressed under a poxvirus synthetic early/late promoter (sE/L), that allows expression only in virus-infected cells. In addition to the multi-specific immune cell engager transgene, a reporter gene, for example green fluorescent protein (GFP) or TdTomato, can also optionally be expressed under a poxvirus promoter for quick selection and purification of transgene-expressing recombinant virus. Additional reporter genes, for example Firefly luciferase (F-Luc) can allow real time monitoring of viral replication in live animals. The transgene and reporter genes can be inserted between the ORFs M135 and M136 of the myxoma virus genome to maintain a parental wild type MYXV backbone. Transgenes can also be inserted in a gene knockout virus background. In this case, the M135 gene locus is selected for construction of a transgene expressing cassette with an M135 knockout. The final recombination plasmid cassette contains: transgene, reporter gene(s) and gene sequences from MYXV where the recombination will take place.

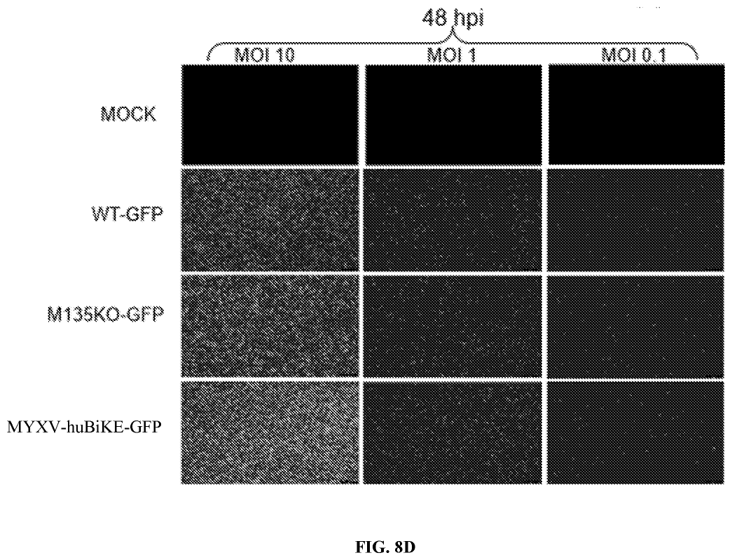

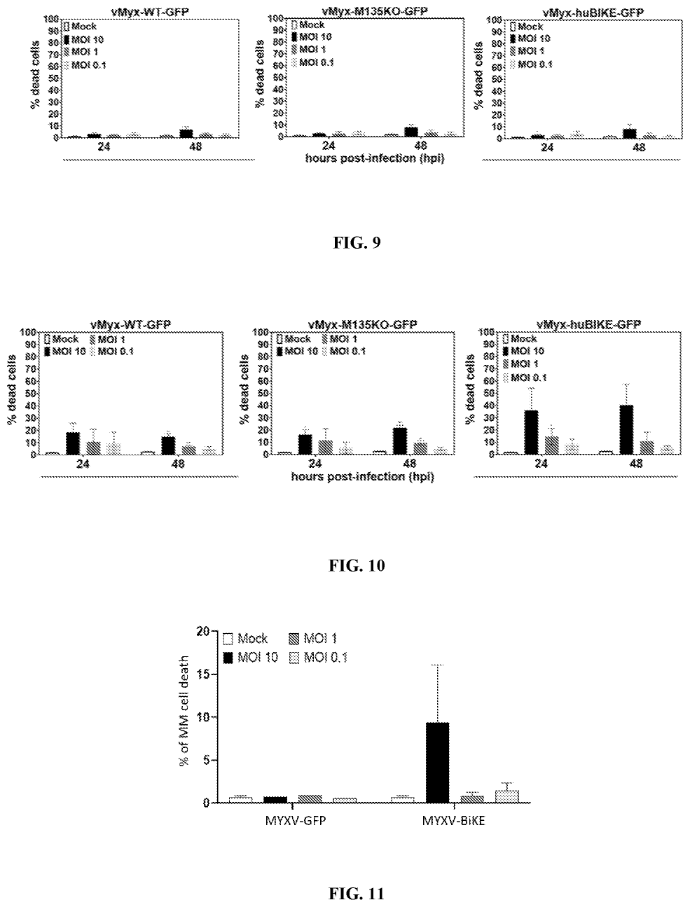

The construction of the recombination plasmids is done by Gateway technology (Multisite Gateway Pro), which allows construction of a single plasmid from multiple DNA fragments by recombination in bacteria. Four entry clones are generated containing different elements to make the final recombination cassette. They are: a) element 1, myxoma virus M135 region; b) element 2, multi-specific immune cell engager with poxvirus Syn E/L promoter sequence and a V5 tag; c) element 3, reporter gene TdTomato under poxvirus late p11 promoter; d) element 4, firefly luciferase under poxvirus Syn E/L promoter, together with the myxoma virus M136 gene sequence. For making the M135KO virus backbone, element 1 is replaced with a partial sequence from the M134 ORF and 50 nt from the M135 ORF. For constructing the final recombination plasmid cassette, all these four elements are recombined with a Gateway destination vector by LR recombination reaction using a standard protocol. The final recombination plasmids are: (i) pDEST M135-136-FLuc-ENGAGER-TdTomato, and (ii) pDEST M135KO-Fluc-ENGAGER-TdTomato, as illustrated in FIG. 12 and FIG. 13 respectively.

At this stage before making the recombinant viruses, the expression of the multi-specific immune cell engager can be confirmed by Western blot analysis (e.g., for the V5 epitope tag) after transfection of the plasmids into RK13 cells.

The final recombination plasmids encoding the transgenes and selection markers together with the flanking sequences are transfected into RK13 cells that are infected with wild type MYXV Lausanne. Recombinant viruses are isolated and serially purified based on the expression of selection marker. Expression of the multi-specific immune cell engager is again confirmed by Western blot analysis and functional assays. Viruses are generated comprising the multi-specific immune cell engager on a wild type virus background, and on a knockout background (in this example, with knockout of M135).

The replication capacity of the MYXV constructs that express the multi-specific immune cell engager can be tested and compared to wild type MYXV, for example, by infecting RK13 cells.

The technique can be adapted to generate knockouts and/or knock-ins at other genetic loci disclosed herein by using alternate flanking sequences. For example, MYXV comprising a deletion or disruption in M153 rather than M135, and expressing one or more multi-specific immune cell engagers can be generated, e.g., by using appropriate flanking sequences for insertion in the M153 gene.

### Example 8: MYXV Expressing Multi-Specific Immune Cell Engagers Enhance Killing of Hematologic Cancer Cells

This example demonstrates evaluating MYXV of the disclosure that express a multi-specific immune cell engager for the ability to kill primary human hematologic cancer cells. Primary blood and bone marrow samples are obtained from patients with hematologic cancers. The samples are subjected to purification using Ficoll-paque plus gradient to isolate mononuclear cells and eliminate the majority of red blood cells (RBCs).

These primary cells in suspension are then mock-treated (i.e., no virus added), or incubated with MYXV of the disclosure at 37° C. for 1 hour to allow virus adsorption. Experiments are conducted at different multiplicities of infection (MOI) including MOI=10, 1, and 0.1. After this, mock-treated, or MYXV-treated cells are incubated overnight (˜24 hours) at 37° C. to allow virus infection.

The percentages of virus infection and the percentages of viability, apoptosis, and cell death of cancer cells are determined using flow cytometry. The percentages are also determined for uninfected cancer cells in patient samples that are exposed to the virus, allowing measurement of cancer cell death in cells that are not directly infected by the virus (i.e. do not express any virus-specific fluorescent protein), but are killed in an “off-target” fashion, e.g., by leukocytes from the same patient samples that are directed to the cancer cells by the multi-specific immune cell engager.

MYXV of the disclosure that express multi-specific immune cell engagers (e.g., MYXV-BiTE, MYXV-BiKE, MYXV-MiTE) productively infects cancer cells. MYXV of the disclosure that express multi-specific immune cell engagers (e.g., MYXV-BiTE, MYXV-BiKE, MYXV-MiTE) directly kills cancer cells that they infect, and promote killing of un-infected cancer cells via the multi-specific immune cell engager.

### Example 9: Multi-Specific Immune Cell Engagers Specifically Bind to Human Cancer Cells and Human Immune Cells

This example demonstrates that multi-specific immune cell engagers of the disclosure specifically bind to human cancer cells and human immune cells.

The MYXV-BiKE, MYXV-BiTE, and/or MYXV-MiTE described in Example 7 are propagated in RK13 cells. The BiKE specifically binds CD16 and CD138. The BiTE specifically binds CD3 and CD13S. The MiTE specifically binds CD3 and CD138. Constructs that also bind other suitable targets disclosed herein can also be generated. Supernatants from MYXV-BiKE-infected RK13 cells, containing secreted BiKE, are harvested. Supernatants from MYXV-BiTE-infected RK13 cells, containing secreted BiTE, are harvested. Supernatants from MYXV-BiTE-infected RK13 cells, containing secreted MiTE, are harvested. As controls, supernatants are also harvested from RK13 cells that are mock infected, or infected with wild type MYXV.

Harvested supernatants are added to cultures of human immune cells, for example, human T cells, human NK cells, or human multiple myeloma (e.g., U266) cells.

To detect BiKE BiTE, or MiTE bound to cells, cells are stained with a PE-conjugated monoclonal antibody specific for the V5 tag at the C-terminus of BiKE/BiTE/MiTE, washed, and analyzed by flow cytometry.

A BiKE with a binding specificity for CD16 and a binding specificity for CD138 exhibits binding to NK cells and multiple myeloma cells.

A BiTE with a binding specificity for CD3 and a binding specificity for CD138 exhibits binding to T cells and multiple myeloma cells.

A MiTE with a binding specificity for CD3 and a binding specificity for CD138 exhibits binding to T cells and multiple myeloma cells.

### Example 10: Multi-Specific Immune Cell Engagers Increase Killing of Human Cancer Cells Co-Cultured with Human Immune Cells

This example demonstrates that multi-specific immune cell engagers of the disclosure can increase killing of human cancer cells co-cultured with human immune cells.

RK13 cells are infected with the MYXV-BiKE, MYXV-BiTE, or MYXV-MiTE described in Example 7 at multiplicities of infection (MOIs) of 1, 5, or 10. The BiKE specifically binds CD16 and CD138. The BiTE specifically binds CD3 and CD138. The MiTE specifically binds CD3 and CD138. Supernatants from the MYXV-BiKE-infected RK13 cells, containing secreted BiKE, supernatants from the MYXV-BiTE-infected RK13 cells, containing secreted BiTE, and supernatants from the MYXV-MiTE-infected RK13 cells, containing secreted MiTE, are harvested. As controls, supernatants are also harvested from RK13 cells that were mock infected.

Harvested supernatants are added to co-cultures containing (i) primary human NK cells and human multiple myeloma MM (U266) cells, or (ii) primary human T cells and MM (U266) cells. After incubation for 24 hours, cells are stained to identify MM cells (CD138+) and dead cells (Near IR stain), then analyzed by flow cytometry.

BiKE, BiTE, and MiTE increase killing of the MM cells by recruiting NK cells and T cells.

### Example 11: Oncolytic Virotherapy with Myxoma Virus (MYXV) Against Multiple Myeloma (MM): Identification of MYXV Constructs Suitable for Eliminating Contaminating Cancer Cells from Primary Human Samples

Experiments are conducted to identify MYXV constructs and experimental conditions suitable for eliminating contaminating refractory cancer cells from primary human cell samples. Bone marrow or peripheral blood samples are obtained from a subject with a hematological cancer (e.g., a myeloma, a leukemia, or a lymphoma). Mononuclear cells are isolated (e.g., via Ficoll-Paque). Samples of mononuclear cells comprising cancer cells are treated with MYXV constructs of the disclosure (e.g., expressing one or more multi-specific immune cell engagers and/or comprising one or more deletions) under various conditions (e.g., MOI, incubation time), and the ability of the MYXV constructs to kill cancer cells is determined as disclosed herein (e.g., via flow cytometry, fluorescence microscopy, and/or cytotoxicity assay).

The identified construct and/or experimental conditions can be used for treating the subject. For example, A MYXV construct identified as suitable can be directly administered to the subject (e.g., via injection or intravenous infusion), or can be administered via MYXV-adsorbed leukocytes.

### Example 12: Oncolytic Virotherapy with a Myxoma Virus (MYXV)

A subject is identified as having a hematological cancer (e.g., a myeloma, leukemia, or lymphoma). The hematological cancer can optionally be a hematological cancer that comprises minimal residual disease (MRD) and/or drug-resistant MRD.

Optionally, studies are conducted to identify a MYXV construct of the disclosure (e.g., expressing one or more multi-specific immune cell engagers and/or comprising one or more deletions) that eliminates cancer cells from a sample taken from the subject (e.g., a peripheral blood or bone marrow sample).

A MYXV is administered to the subject (e.g., administered via injection or infusion). The MYXV infects cancer cells in the subject and expresses the multi-specific immune cell engager, leading to cancer cell killing and an anti-cancer immune response.

### Example 13: Oncolytic Virotherapy with Myxoma Virus (MYXV) Via Autologous Transplant of MYXV-Adsorbed Leukocytes

A MYXV is administered to a subject with a hematological cancer via autologous transplant of MYXV-adsorbed leukocytes.

Bone marrow or peripheral blood samples are obtained from a subject with a hematological cancer (e.g., a myeloma, leukemia, or lymphoma), and mononuclear cells are isolated (e.g., via Ficoll-Paque). Cancer cells can be separated from non-cancer cells (e.g., via FACS or MACS). A MYXV of the disclosure is adsorbed to leukocytes (for example, adsorbed for about an hour at an MOI of about 0.1 to 10). The MYXV-adsorbed leukocytes are administered back to the subject via intravenous infusion. The MYXV infects cancer cells in the subject and expresses the multi-specific immune cell engager, leading to cancer cell killing and an anti-cancer immune response.

### Example 14: Oncolytic Virotherapy with Myxoma Virus (MYXV) Via Allogenic Transplant of MYXV-Adsorbed Leukocytes

A MYXV is administered to a subject with a hematological cancer (e.g., a myeloma, leukemia, or lymphoma) via allogenic transplant of MYXV-adsorbed leukocytes. Bone marrow or peripheral blood samples are obtained from a donor (e.g., an HLA-matched, HLA-mismatched, haploidentical, or sibling donor, or a combination thereof). Mononuclear cells are isolated (e.g., via Ficoll-Paque). Optionally, cells are purified or enriched for specific leukocyte subsets (e.g., via FACS or MACS). A MYXV of the disclosure is adsorbed to leukocytes (for example, adsorbed for about an hour at an MOI of about 0.1 to 10). The MYXV-adsorbed leukocytes are administered back to the subject via intravenous infusion. The MYXV infects cancer cells in the subject and expresses the multi-specific immune cell engager, leading to cancer cell killing and an anti-cancer immune response.

### Example 15: MYXV-Adsorbed Primary Human PBMCs Transfer MYXV to Susceptible MM Cells

PBMCs from primary human peripheral blood from healthy patients were first isolated using Ficoll-Paque PLUS gradient. NK cells were isolated from these PBMCs using MACS human NK cell isolation kit and LS magnetic columns to deplete magnetically labeled cells. The NK cell-depleted fraction was retained and used separately. 1×10\hat{ }6 NK cells or PBMCs depleted of NK cells were incubated with MYXV (MYXV-GFP or MYXV-BIKE-GFP) in 380 μL complete media per condition in 24-well plates at 37° C. for 1 hour to allow virus adsorption. After the 1 h of incubation, the primary cells were washed three times using 500 μL 1×PBS+10% FBS to remove unbound virus.

The primary cells were then resuspended in 500 μL complete media containing 2×10\hat{ }5 U266 cells (CD138+ MM cells). After co-incubating for 24 hours, the cells were labeled with near-IR LIVE/DEAD. The cells were subsequently labeled with 1 μL human anti-CD138 antibody in 100 μL staining buffer per condition and incubated for 15 minutes at 4° C., protected from light. All samples were then fixed using 100 μL Cytofix and incubated for 15 minutes at 4° C. with light protection, before resuspending in 270 μL staining buffer for flow cytometry analysis.

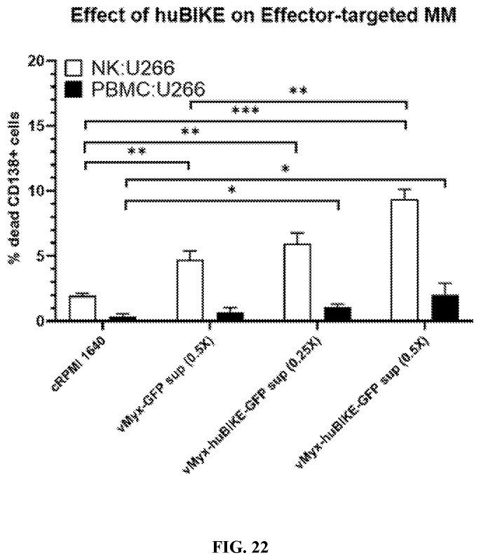

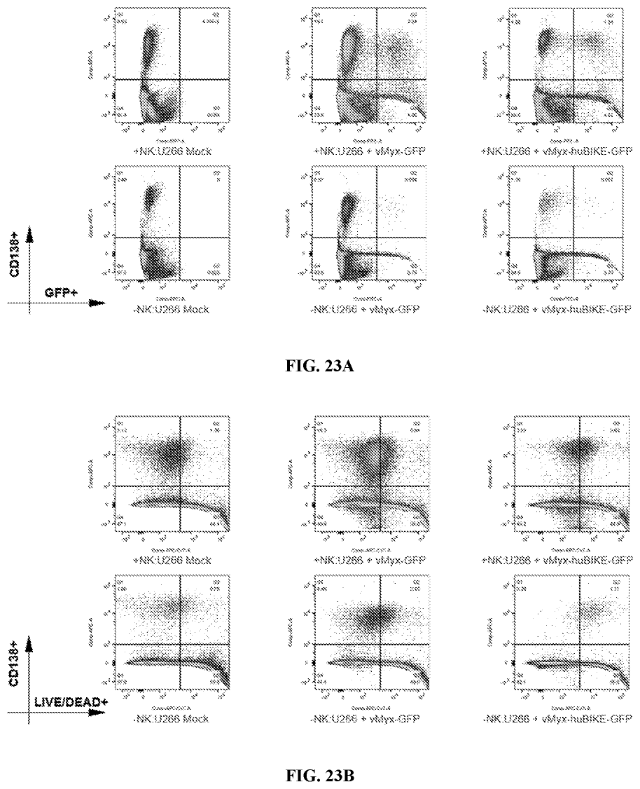

FIG. 23A provides dot-plots that demonstrate infection of CD138+ MM cells after co-incubation with MYXV-GFP or MYXV-BiKE-adsorbed NK cells (top row) or NK-depleted PBMCs (−NK, bottom row). These data demonstrate that virus-adsorbed NK cells or PBMCs can deliver a MYXV of the disclosure to human hematologic cancer cells, which the MYXV can then infect.

FIG. 23B provides dot-plots that demonstrate killing of CD138+ MM cells after co-incubation with MYXV-GFP or MYXV-BiKE-adsorbed NK cells (top row) or NK-depleted PBMCs (−NK, bottom row). These data demonstrate that virus-adsorbed NK cells or PBMCs can deliver a MYXV of the disclosure to human hematologic cancer cells, which the MYXV can then infect and kill.

### Example 16: Ex Vivo MYXV Virotherapy in Conjunction with Auto-Transplants in the Vk*MYC Immunocompetent Mouse Model of Minimal Residual Disease (MRD) to Target and Eliminate Drug-Resistant Disseminated MM In Vivo

Two C57BL/6-derived VK*MYC cell lines were used for in vivo experiments: VK12598, which is bortezomib-resistant (BOR-resistant), and the multi-drug resistant line VK12653. First, the susceptibility of these two VK*MYC cell lines to MYXV binding and infection was evaluated.

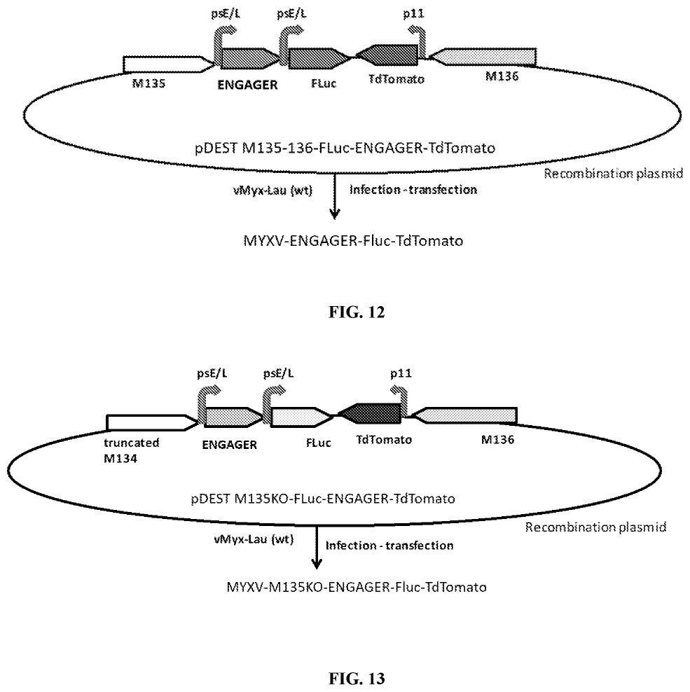

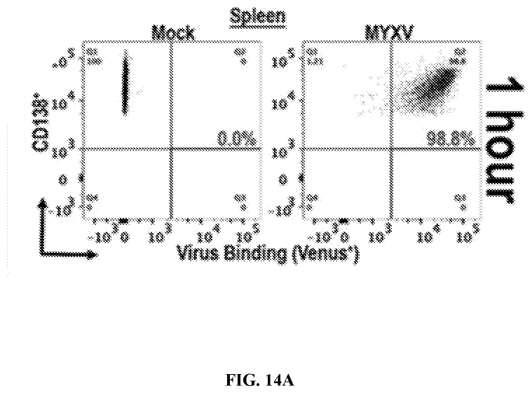

MYXV binding to VK12598 and VK12653, in vitro studies: For binding experiments, MYXV-M093L-Venus virus (comprising a fusion of the fluorescent protein Venus at the amino terminus of M093L) was used at a multiplicity of infection (MOI) of 10. In brief, either VK12598, or VK12653 were freshly isolated from BM (or from freshly-thawed BM), and incubated with MYXV-M093L-Venus at 4° C. for 1 hour to allow virus binding. Unbound virus was removed by washing the virus-adsorbed cells twice. Levels of virion binding were quantified using flow cytometry. For analyses of virus infection, cells were incubated with reporter MYXV-GFP(E/L)/TdTomato(L) at MOI=10 for 1 hour at 37° C. to allow virus adsorption. Cells were incubated overnight at 37° C. to allow virus infection. MYXV efficiently bound to both cell lines (FIGS. 14A and 15A). In addition to this, MYXV productively infects both cell lines (FIGS. 14B-C and 15B).

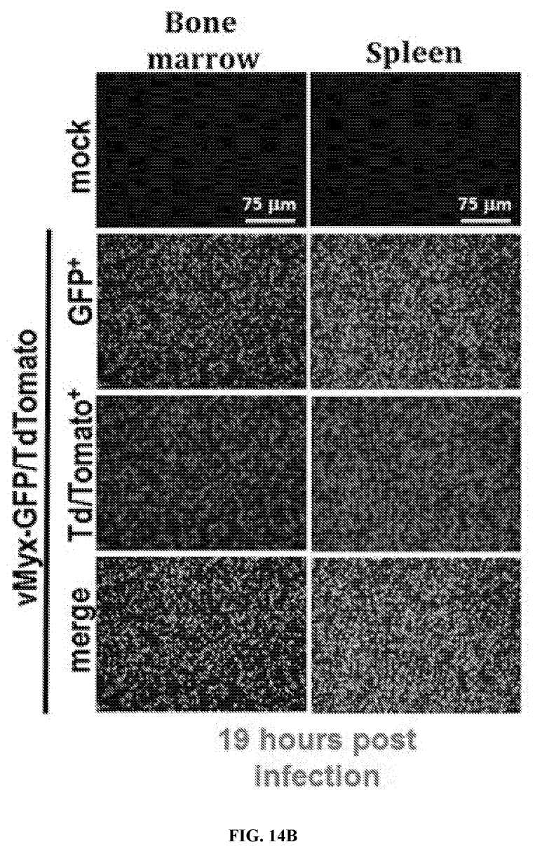

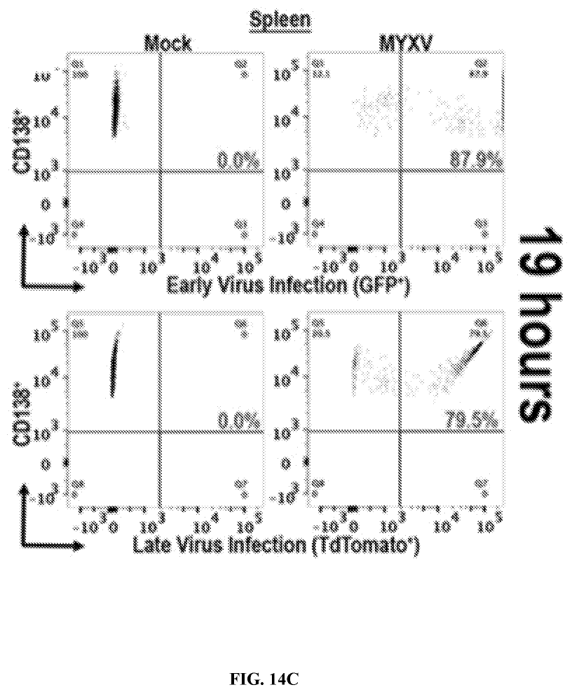

In vivo studies using the VK12598 cell line: In the first in vivo experiment, C57BL/6 mice were pre-seeded with VK12598 cells (e.g., 1×10⁶ cells per mouse). Four weeks post-MM cell implantation, mice were subjected to bleeding and the M-Spike was measured. Mice were separated according to the levels of M-Spike (e.g., 0, low=0.1, medium=0.2, high=0.6) (FIG. 16A, Top panel). Mice were then treated as follows: No C57BL/6 BM transplant (Cohort I), C57BL/6 BM cells alone (Cohort II), MYXV-M135KO-GFP alone (Cohort III), C57BL/6 BM ex vivo treated with MYXV-M135KO-GFP (Cohort IV) (FIG. 16A, bottom panel). FIG. 16B shows the percentage of MM (CD138⁺B220⁻) in a representative mouse from Cohort I with low M-spike (0.1) and the percentage of MM (CD138⁺B220⁻) in a representative mouse from Cohort II with high M-spike (0.6). FIG. 16C shows the M-spike of the only survivor from Cohort IV, which exhibited total regression of MM, with no M-spike band detected on day 8, day 29, and day 37 post-transplant. These data indicate that a transplant of ex vivo MYXV-treated bone marrow can induce MM regression. Together, these data may suggest that the cohort treatment in this first experiment started too late in the disease progression, and instead virotherapy should be started much earlier in this model (e.g. less than 1 week post-MM implantation rather than 4 weeks post-MM implantation). Although MM regression can occur even at this late intervention time, starting virotherapy earlier (e.g., in mouse cohorts that are not so close to death or end-point) may allow improved evaluation of the virus technology.

In additional trial, MYXV is tested in combination with other therapeutics (such as the SMAC mimetic LC161). VK12598 cancer cells are implanted, M-Spike quantified at 1-4 weeks, and the mice are treated with cyclophosphamide to induce a transient complete response (CR), which can last 1 month. At either one or two weeks post cyclophosphamide, the mice are transplanted with BM+MYXV or PBMC+MYXV (e.g., MYXV expressing an immune cell engager as disclosed herein) in order to test if the virotherapy can extend or complete the partial regression initiated by the cyclophosphamide. In this setting, the capacity of MYXV to eliminate MM minimal residual disease (MRD) as defined by disease that functionally resists this chemotherapy is investigated. The capacity of MYXV to eliminate the multidrug-resistant VK12653 cells line, either as a monotherapy or in combination therapy is also investigated.

While this disclosure has been described with an emphasis upon particular embodiments, it will be obvious to those of ordinary skill in the art that variations of the particular embodiments may be used, and it is intended that the disclosure may be practiced otherwise than as specifically described herein. Features, characteristics, compounds, or examples described in conjunction with a particular aspect, embodiment, or example of the invention are to be understood to be applicable to any other aspect, embodiment, or example of the invention. Accordingly, this disclosure includes all modifications encompassed within the spirit and scope of the disclosure as defined by the following claims. We therefore claim as our invention all that comes within the scope and spirit of these claims.

### Embodiments

Embodiment 1. A myxoma virus (MYXV) comprising a transgene that encodes a multi-specific immune cell engager.

Embodiment 2. The myxoma virus of embodiment 1, wherein the multi-specific immune cell engager is a Bi-specific Natural Killer and Neutrophil engager (BiKE).

Embodiment 3. The myxoma virus of embodiment 2, wherein the BiKE binds to an antigen present on a natural killer cell, a neutrophil, or a combination thereof.

Embodiment 4. The myxoma virus of embodiment 2 or embodiment 3, wherein the BiKE binds to an antigen present on a hematologic cancer cell.

Embodiment 5. The myxoma virus of any one of embodiments 2-4, wherein the BiKE binds to an antigen present on a myeloma cell.

Embodiment 6. The myxoma virus of any one of embodiments 2-5, wherein the BiKE binds to an antigen present on a leukemia cell.

Embodiment 7. The myxoma virus of any one of embodiments 2-6, wherein the BiKE binds to an antigen present on a lymphoma cell.

Embodiment 8. The myxoma virus of any one of embodiments 2-7, wherein the BiKE binds to CD16.

Embodiment 9. The myxoma virus of any one of embodiments 2-8, wherein the BiKE binds CD138.

Embodiment 10. The myxoma virus of any one of embodiments 2-9, wherein the BiKE comprises one or more single chain variable fragments (scFvs).

Embodiment 11. The myxoma virus of any one of embodiments 2-9, wherein the BiKE comprises one or more humanized single chain variable fragments (scFvs).

Embodiment 12. The myxoma virus of any one of embodiments 2-11, wherein the BiKE comprises a sequence that is at least 70%, at least 80%, at least 90%, at least 95%, at least 97%, at least 98%, at least 99%, or 100% identical to any one of SEQ ID NOs: 4-21.

Embodiment 13. The myxoma virus of any one of embodiments 2-12, wherein the BiKE is between the M135 and M136 open reading frames of the myxoma virus genome.

Embodiment 14. The myxoma virus of any one of embodiments 1-13, further comprising a reporter gene.

Embodiment 15. The myxoma virus of embodiment 14, wherein the reporter gene is a fluorescent protein.

Embodiment 16. The myxoma virus of embodiment 14, wherein the reporter gene is a luminescent substrate or enzyme.

Embodiment 17. The myxoma virus of any one of embodiments 1-16, further comprising a deletion in the myxoma virus genome.

Embodiment 18. The myxoma virus of embodiment 17, wherein the myxoma virus comprises a deletion or disruption of one or more genes selected from the group consisting of M001R, M002R, M003.1R, M003.2R, M004.1R, M004R, M005R, M006R, M007R, M008.1R, M008R, M009L, M013, M036L, M063L, M11L, M128L, M131R, M135R, M136R, M141R, M148R, M151R, M152R, M153R, M154L, M156R, M-T2, M-T4, M-T5, M-T7, and SOD.

Embodiment 19. The myxoma virus of embodiment 17, wherein the myxoma virus comprises a deletion of M135.

Embodiment 20. The myxoma virus of embodiment 1, wherein the multi-specific immune cell engager is a Bi-specific T Cell Engager (BiTE).

Embodiment 21. The myxoma virus of embodiment 20, wherein the BiTE binds to an antigen present on a T cell.

Embodiment 22. The myxoma virus of embodiment 20 or embodiment 21, wherein the BiTE binds to an antigen present on a hematologic cancer cell.

Embodiment 23. The myxoma virus of any one of embodiments 20-22, wherein the BiTE binds to an antigen present on a myeloma cell.

Embodiment 24. The myxoma virus of any one of embodiments 20-23, wherein the BiTE binds to an antigen present on a leukemia cell.

Embodiment 25. The myxoma virus of any one of embodiments 20-24, wherein the BiTE binds to an antigen present on a lymphoma cell.

Embodiment 26. The myxoma virus of any one of embodiments 20-25, wherein the BiTE binds to CD3.

Embodiment 27. The myxoma virus of any one of embodiments 20-26, wherein the BiTE binds CD138.

Embodiment 28. The myxoma virus of any one of embodiments 20-27, wherein the BiTE comprises one or more single chain variable fragments (scFvs).

Embodiment 29. The myxoma virus of any one of embodiments 20-28, wherein the BiTE comprises one or more humanized single chain variable fragments (scFvs).

Embodiment 30. The myxoma virus of any one of embodiments 20-29, wherein the BiTE comprises a sequence that is at least 70%, at least 80%, at least 90%, at least 95%, at least 97%, at least 98%, at least 99%, or 100% identical to any one of SEQ ID NOs: 6, 7, 10-15, or 32-39.

Embodiment 31. The myxoma virus of any one of embodiments 20-30, wherein the BiTE is between the M135 and M136 open reading frames of the myxoma virus genome.

Embodiment 32. The myxoma virus of any one of embodiments 20-31, further comprising a reporter gene.

Embodiment 33. The myxoma virus of embodiment 32, wherein the reporter gene is a fluorescent protein.

Embodiment 34. The myxoma virus of embodiment 32, wherein the reporter gene is a luminescent substrate or enzyme.

Embodiment 35. The myxoma virus of any one of embodiments 20-34, further comprising a deletion in the myxoma virus genome.

Embodiment 36. The myxoma virus of any one of embodiments 20-34, wherein the myxoma virus comprises a deletion or disruption of one or more genes selected from the group consisting of M001R, M002R, M003.1R, M003.2R, M004.1R, M004R, M005R, M006R, M007R, M008.1R, M008R, M009L, M013, M036L, M063L, M11L, M128L, M131R, M135R, M136R, M141R, M148R, M151R, M152R, M153R, M154L, M156R, M-T2, M-T4, M-T5, M-T7, and SOD.

Embodiment 37. The myxoma virus of any one of embodiments 20-34, wherein the myxoma virus comprises a deletion of M135.

Embodiment 38. The myxoma virus of embodiment 1, wherein the multi-specific immune cell engager is a membrane-integrated T cell engager (MiTE).

Embodiment 39. The myxoma virus of embodiment 38, wherein the MiTE binds to an antigen present on a T cell.

Embodiment 40. The myxoma virus of embodiment 38 or embodiment 39, wherein the MiTE binds to an antigen present on a hematologic cancer cell.

Embodiment 41. The myxoma virus of any one of embodiments 38-40, wherein the MiTE binds to an antigen present on a myeloma cell.

Embodiment 42. The myxoma virus of any one of embodiments 38-41, wherein the MiTE binds to an antigen present on a leukemia cell.

Embodiment 43. The myxoma virus of any one of embodiments 38-42, wherein the MiTE binds to an antigen present on a lymphoma cell.

Embodiment 44. The myxoma virus of any one of embodiments 38-43, wherein the MiTE binds to CD3.

Embodiment 45. The myxoma virus of any one of embodiments 38-44, wherein the MiTE binds CD138.

Embodiment 46. The myxoma virus of any one of embodiments 38-45, wherein the MiTE comprises one or more single chain variable fragments (scFvs).

Embodiment 47. The myxoma virus of any one of embodiments 38-45, wherein the MiTE comprises one or more humanized single chain variable fragments (scFvs).

Embodiment 48. The myxoma virus of any one of embodiments 38-47, wherein the MiTE comprises a sequence that is at least 70%, at least 80%, at least 90%, at least 95%, at least 97%, at least 98%, at least 99%, or 100% identical to any one of SEQ ID NOs: 6, 7, 10-15, or 32-39.

Embodiment 49. The myxoma virus of any one of embodiments 38-48, wherein the MiTE is between the M135 and M136 open reading frames of the myxoma virus genome.

Embodiment 50. The myxoma virus of any one of embodiments 38-49, further comprising a reporter gene.

Embodiment 51. The myxoma virus of embodiment 50, wherein the reporter gene is a fluorescent protein.

Embodiment 52. The myxoma virus of embodiment 50, wherein the reporter gene is a luminescent substrate or enzyme.

Embodiment 53. The myxoma virus of any one of embodiments 38-52, further comprising a deletion in the myxoma virus genome.

Embodiment 54. The myxoma virus of any one of embodiments 38-53, wherein the myxoma virus comprises a deletion or disruption of one or more genes selected from the group consisting of M001R, M002R, M003.1R, M003.2R, M004.1R, M004R, M005R, M006R, M007R, M008.1R, M008R, M009L, M013, M036L, M063L, M11L, M128L, M131R, M135R, M136R, M141R, M148R, M151R, M152R, M153R, M154L, M156R, M-T2, M-T4, M-T5, M-T7, and SOD.

Embodiment 55. The myxoma virus of any one of embodiments 38-53, wherein the myxoma virus comprises a deletion of M135.

Embodiment 56. A composition comprising the myxoma virus of any one of embodiments 1-55 and a pharmaceutically acceptable carrier.

Embodiment 57. A method of treating a hematological cancer in a subject in need thereof, comprising administering to the subject the myxoma virus of any one of embodiments 1-56.

Embodiment 58. The method of embodiment 57, wherein the subject is a human.

Embodiment 59. The method of embodiment 57 or embodiment 58, wherein the myxoma virus is capable of infecting cells that have a deficient innate anti-viral response.

Embodiment 60. The method of any one of embodiments 57-59, wherein the myxoma virus is capable of infecting cancer cells.

Embodiment 61. The method of any one of embodiments 57-60, wherein the hematological cancer is a myeloma, leukemia, or lymphoma.

Embodiment 62. The method of any one of embodiments 57-60, wherein the hematological cancer is multiple myeloma.

Embodiment 63. A method of treating a hematological cancer in a subject in need thereof, comprising administering to the subject a leukocyte, wherein the leukocyte comprises the myxoma virus of any one of embodiments 1-56.

Embodiment 64. The method of embodiment 63, further comprising adsorbing the myxoma virus ex vivo onto a surface of the leukocyte.

Embodiment 65. The method of embodiment 64, wherein the adsorbing the myxoma virus onto the surface of the leukocyte comprises exposing the leukocyte to the myxoma virus under conditions that permit binding of the myxoma virus to the surface of the leukocyte.

Embodiment 66. The method of embodiment 64 or embodiment 65, wherein the myxoma virus is exposed to the leukocyte for at least five minutes.

Embodiment 67. The method of embodiment 64 or embodiment 65, wherein the myxoma virus is exposed to the leukocyte for about one hour.

Embodiment 68. The method of any one of embodiments 64-67, wherein the myxoma virus is exposed to the leukocyte at a multiplicity of infection (MOI) of between about 0.001 and 1000.

Embodiment 69. The method of any one of embodiments 64-67, wherein the myxoma virus is exposed to the leukocyte at a multiplicity of infection (MOI) of between about 0.1 and 10.

Embodiment 70. The method of any one of embodiments 63-69, wherein the leukocyte is obtained from peripheral blood.

Embodiment 71. The method of any one of embodiments 63-69, wherein the leukocyte is obtained from bone marrow.

Embodiment 72. The method of any one of embodiments 63-69, wherein the leukocyte is a peripheral blood mononuclear cell.

Embodiment 73. The method of any one of embodiments 63-72, wherein the leukocyte is obtained from the subject.

Embodiment 74. The method of any one of embodiments 63-73, wherein the leukocyte is obtained from a donor that is HLA-matched, HLA-mismatched, haploidentical, or a combination thereof relative to the subject.

Embodiment 75. The method of any one of embodiments 63-74, wherein the leukocyte is administered in a pharmaceutical composition.

Embodiment 76. The method of any one of embodiments 63-75, wherein the leukocyte is administered systemically.

Embodiment 77. The method of any one of embodiments 63-76, wherein the leukocyte is administered parenterally.

Embodiment 78. The method of any one of embodiments 63-77, wherein the leukocyte is administered by infusion.

What is claimed is:

1. A myxoma virus (MYXV) comprising a transgene that encodes a multi-specific immune cell engager.

2. The MYXV of claim 1, wherein the multi-specific immune cell engager comprises a Bi-specific Natural Killer and Neutrophil engager (BiKE), a Bi-specific T Cell Engager (BiTE), or a membrane-integrated T cell engager (MiTE).

3. The MYXV of claim 1 or claim 2, wherein the multi-specific immune cell engager binds to an antigen present on a hematologic cancer cell.

4. The MYXV of claim 3, wherein the hematologic cancer cell is a myeloma cell, a leukemia cell, or a lymphoma cell.

5. The MYXV of claim 2, wherein the BiKE binds to an antigen present on a natural killer cell or a neutrophil.

6. The MYXV of claim 2, wherein the BiTE binds to an antigen present on a T cell.

7. The MYXV of claim 2, wherein the MiTE binds to an antigen present on a T cell.

8. The MYXV of any one of claims 2-7, wherein the BiKE binds to CD16 or CD138.

9. The MYXV of any one of claims 2-8, wherein the BiKE binds to CD16 and CD138.

10. The MYXV of any one of claims 2-8, wherein the BiTE binds to CD3 or CD138.

11. The MYXV of any one of claims 2-8, wherein the BiTE binds to CD3 and CD138.

12. The MYXV of any one of claims 2-8, wherein MiTE binds to CD3 or CD138.

13. The MYXV of any one of claims 2-8, wherein MiTE binds to CD3 and CD138.

14. The MYXV of any one of claims 1-13, wherein the multi-specific immune cell engager comprises one or more single chain variable fragments (scFvs) derived from an anti-human CD antibody.

15. The MYXV of any one of claims 1-13, wherein the multi-specific immune cell engager comprises one or more humanized single chain variable fragments (scFvs).

16. The MYXV of any one of claims 2-15, wherein the BiKE comprises a sequence that is at least 70%, at least 80%, at least 85%, at least 90%, at least 95%, at least 97%, at least 98%, at least 99%, or 100% identical to any one of SEQ ID NOs: 4-21.

17. The MYXV of any one of claims 2-15, wherein the BiTE comprises a sequence that is at least 70%, at least 80%, at least 85%, at least 90%, at least 95%, at least 97%, at least 98%, at least 99%, or 100% identical to any one of SEQ ID NOs: 6, 7, 10-15, or 32-39.

18. The MYXV of any one of claims 2-15, wherein the MiTE comprises a sequence that is at least 70%, at least 80%, at least 85%, at least 90%, at least 95%, at least 97%, at least 98%, at least 99%, or 100% identical to any one of SEQ ID NOs: 6, 7, 10-15, or 32-39.

19. The MYXV of any one of claims 1-18, wherein the transgene is located between the M135 gene and M136 gene of the genome of the MYXV.

20. The MYXV of any one of claims 1-19, further comprising a reporter gene.

21. The MYXV of claim 20, wherein the reporter gene encodes a fluorescent protein.

22. The MYXV of claim 20, wherein the reporter gene encodes a luminescent substrate or an enzyme.

23. The MYXV of any one of claims 1-22, further comprising a mutation in the genome of the MYXV.

24. The MYXV of claim 23, wherein the mutation is present in one or more genes selected from the group consisting of M001R, M002R, M003.1R, M003.2R, M004.1R, M004R, M005R, M006R, M007R, M008.1R, M008R, M009L, M013, M036L, M063L, M11L, M128L, M131R, M135R, M136R, M141R, M148R, M151R, M152R, M153R, M154L, M156R, M-T2, M-T4, M-T5, M-T7, and SOD.

25. The MYXV of claim 23 or claim 24, wherein the mutation is a deletion.

26. The MYXV of claim 25, wherein the deletion deletes at least a portion of M135R.

27. The MYXV of any one of claims 1-26, wherein the MYXV increases killing of infected cancer cells by at least 5% compared to a MYXV that lacks the transgene as determined by an in vitro flow cytometric assay.

28. The MYXV of any one of claims 1-27, wherein the MYXV increases killing of uninfected cancer cells by at least 5% compared to a MYXV that lacks the transgene as determined by an in vitro flow cytometric assay.

29. A composition comprising the MYXV of any one of claims 1-28 and a pharmaceutically acceptable carrier.

30. A method of treating a hematological cancer in a subject in need thereof, comprising administering to the subject the MYXV of any one of claims 1-28 or the composition of claim 29.

31. The method of claim 30, wherein the subject is a human.

32. The method of claim 30 or claim 31, wherein the MYXV is capable of infecting cells that have a deficient innate anti-viral response.

33. The method of any one of claims 30-32, wherein the MYXV is capable of infecting cancer cells.

34. The method of any one of claims 30-33, wherein the hematological cancer is a myeloma, multiple myeloma, leukemia, or lymphoma.

35. A method of treating a hematological cancer in a subject in need thereof, comprising administering to the subject a leukocyte, wherein the leukocyte comprises or is associated with the MYXV of any one of claims 1-28.

36. The method of claim 35, further comprising adsorbing the MYXV ex vivo onto a surface of the leukocyte.

37. The method of claim 36, wherein the adsorbing the MYXV onto the surface of the leukocyte comprises exposing the leukocyte to the MYXV under conditions that permit binding of the MYXV to the surface of the leukocyte.

38. The method of claim 36 or claim 37, wherein the adsorbing comprises exposing the leukocyte to the MYXV for at least five minutes.

39. The method of claim 36 or claim 37, wherein adsorbing comprises exposing the leukocyte to the MYXV for about one hour.

40. The method of any one of claims 36-39, wherein the adsorbing comprises exposing the leukocyte to the MYXV at a multiplicity of infection (MOI) of between about 0.001 and 1000.

41. The method of any one of claims 36-39, wherein the adsorbing comprises exposing the leukocyte to the MYXV at a multiplicity of infection (MOI) of between about 0.1 and 10.

42. The method of any one of claims 36-41, wherein the leukocyte is obtained from peripheral blood.

43. The method of any one of claims 36-42, wherein the leukocyte is obtained from bone marrow.

44. The method of any one of claims 36-42, wherein the leukocyte is a peripheral blood mononuclear cell.

45. The method of any one of claims 36-44, wherein the leukocyte is obtained from the subject's tissue.

46. The method of any one of claims 36-44, wherein the leukocyte is obtained from a donor's tissue that is HLA-matched, HLA-mismatched, haploidentical, or a combination thereof relative to the subject.

47. The method of any one of claims 36-46, wherein the leukocyte is formulated in a pharmaceutical composition.

48. The method of any one of claims 36-47, wherein the leukocyte is administered systemically.

49. The method of any one of claims 36-47, wherein the leukocyte is administered parenterally.

50. The method of any one of claims 36-47, wherein the leukocyte is administered intravenously.
